SEQUENCE LISTING (8 sequences)

SEQ ID NO: 1 - protein, 1206 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 2 - protein, 110 aa, Artificial Sequence
  lambda bacteriophage capsid protein
MTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIV

SEQ ID NO: 3 - protein, 12 aa, Artificial Sequence
  synthetic polypeptide
TSGSGSGSGSGT

SEQ ID NO: 4 - DNA, 3759 bp, Homo sapiens
cagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaataagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggtgcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatg

SEQ ID NO: 5 - DNA, 330 bp, artificial sequence
  gpD capsid protein DNA sequence
atgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtt

SEQ ID NO: 6 - DNA, 51 bp, Artificial Sequence
  linker sequence
actagcggttccggttctggttccggttctggttccggttctggcggtacc

SEQ ID NO: 7 - DNA, 4140 bp, Artificial Sequence
  gpD- linker-EGF nucleotide sequence
atgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgttactagcggttccggttctggttccggttctggttccggttctggcggtacccagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaataagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggtgcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatg

SEQ ID NO: 8 - protein, 17 aa, Artificial Sequence
  linker sequence
TSGSGSGSGSGSGSGGT

(19) United States
(12) Patent Application Publication
(10) Pub. No.: US 2021/0100858 A1
(43) Pub. Date: Apr. 8, 2021

(54) EGFR BINDING MOIETY-PRESENTING BACTERIOPHAGES FOR TUMOUR TREATMENT

(71) Applicant: Theraphage Inc., Kitchener (CA)

(72) Inventors: Roderick A. SLAVCEV, Kitchener (CA); Haein HUH, Kitchener (CA); Jonathan BLAY, Kitchener (CA)

(21) Appl. No.: 17/065,814

(22) Filed: Oct. 8, 2020

Related U.S. Application Data
(60) Provisional application No. 62/912,224, filed on Oct. 8, 2019.

(51) Int. Cl. A61K 35/768; C12N 7/00; C07K 14/485

(52) U.S. Cl. CPC A61K 35/768; C12N 2795/00021; C07K 14/485; C12N 7/00

(57) ABSTRACT

Disclosed herein is a targeted tumour-infiltrating bacteriophage. The bacteriophage is engineered to present an epidermal growth factor receptor (EGFR)-binding moiety on the bacteriophage cell surface. The EGFR-binding moiety is capable of binding the extracellular domain of the EGFR. Also disclosed are compositions, kits, methods and uses thereof. Also disclosed is a method of treating a tumour in a subject in need thereof, the method comprising: administering to the subject a composition comprising a plurality of bacteriophage engineered to present an epidermal growth factor receptor (EGFR)-binding moiety on the bacteriophage cell surface in a dose effective to treat the tumour, wherein the tumour is an EGFR-positive tumour and the EGFR-binding moiety is capable of binding an EGFR extracellular domain.

### CROSS REFERENCE TO RELATED APPLICATIONS

This application claims the benefit of priority of U.S. Provisional Patent Application No. 62/912,224 filed Oct. 8, 2019, which is hereby incorporated by reference. All publications, patents, and patent applications mentioned in this specification and exhibits are herein incorporated by reference to the same extent as if each individual publication, patent, or patent application is specifically and individually indicated to be incorporated by reference.

The instant application contains a Sequence Listing which has been submitted electronically in ASCII format and is hereby incorporated by reference in its entirety. Said ASCII copy, created on Oct. 8, 2020, is named PAT_109392-2_SL.txt and is 23,506 bytes in size.

### FIELD

The present disclosure relates generally to targeted tumour infiltrating bacteriophages and their uses. More particularly, the present disclosure relates to bacteriophages modified to present an epidermal growth factor receptor-binding moiety on the bacteriophage cell surface and methods of treating an epidermal growth factor receptor-positive tumour.

### BACKGROUND

Solid tumours are characterized by a complex structure comprised of extracellular matrix, neoplastic cells and stromal cells, each presenting a barrier to conventional anticancer chemotherapy as well as carrier-mediated drug delivery. Poor penetration of therapeutics into the interstitial tumour microenvironment remains a challenge, with drugs accumulating primarily in the regions of tumours that are situated close to blood vessels.

Drug delivery technology has advanced in the recent years; however, the development of an efficient delivery system into solid tumours is still much needed. Ideally, this system would confer: i) specific targeting to minimize drug-associated systemic toxic effects; ii) effective extravasation into the tumour interstitium; iii) effective diffusion through the dense tumour extracellular matrix; and iv) internalization into desired cells to deliver drug payload. Various nanocarriers have been examined for their potential to improve the bioavailability and preferential accumulation in the tumour site. Oftentimes, these particles are found situated closest to the vessel from which they extravasated from in vivo and do not achieve effective infiltration into the tumour.

Solid tumours and the tumour microenvironment: The complex and dynamic processes of tumourigenesis highlight the need for the development of effective treatment. Different cellular and non-cellular elements of the tumour microenvironment have hindered the applications of emerging antineoplastic, low-weight molecules. Cellular resistance to anticancer drugs is caused by mechanisms such as drug export, changes in drug metabolism and mutations in drug targets that activate survival signaling pathways or inactivate death signaling pathways (Gottesman, 2002). Effective drug delivery adds another layer of complexity in treatment due to the pathophysiology of the tumour microenvironment. This includes irregular vasculature that generates considerable variations throughout the tumour microenvironment, creating regions that are hypoxic and poorly perfused. Different exposures to nutrients and oxygen cause variable proliferation rates in cells that then respond differently to drugs. To achieve sufficient cytotoxicity in tumour cells, systemically administered anti-cancer drugs need to reach the tumour vasculature network, extravasate across the vessel into the interstitial space and reach each tumour cell at a pharmacologically-effective dose. For successful cancer treatment, all tumour cells must be accessible by the drug, otherwise survival of a few cells could lead to tumour recurrence for the patient. This is difficult for most conventional chemotherapeutic drugs to achieve. Hence, much attention has been directed to the development of novel drug delivery methods for which optimal surface physio-chemical properties can be conferred to maximize tumour accumulation. Components of the tumour microenvironment that impede the therapeutic course of drug delivery are further discussed below.

Extracellular matrix (ECM): A major component of the tumour stroma is the extracellular matrix (ECM), which is a network of collagen, elastin fibers, proteoglycans and hyaluronic acid present in the basement membrane and the looser matrix of the interstitial space. ECM provides structural integrity for tissues, modulates cell function, and serves as reservoirs for growth factors and signaling molecules. ECM components, however, will physically impede immune cells and therapeutic drugs from effectively penetrating into the tumour. Furthermore, ECM composition becomes altered as proteins and growth modulators become overexpressed in the tumour microenvironment. Such molecules include osteoblast-specific factor 2 (OSF-2), which is involved in cell adhesion; versican, a large chondroitin sulfate proteoglycan; and hyaluronan, a glycosaminoglycan that is often deposited in solid tumour and has been implicated in epithelial-to-mesenchymal transition (EMT) and drug resistance. Notably, collagen, a protein of the connective tissue that provides tensile strength, increases in its deposition over time with tumour progression and serves as a primary barrier against drug penetration. Collagen accumulation has been associated with ECM remodeling, which is caused by the aberrant expression of the enzyme lysyl oxidase (LOX). LOX mediates collagen cross-linking resulting in enhanced tumour cell survival, metastasis and overall tumour stiffness. Netti et al. demonstrated the correlation between an extended collagen network and poor penetration of model proteins, IgG and BSA, using xenografted tumours from four different tumour lines: human colon adenocarcinoma (LS174T), human glioblastoma (U87), human soft tissue sarcoma (HTS 26T) and murine mammary carcinoma (MCalV). Accumulation of these proteins is poorer in U87 and HTS26T tumours. Histological staining showed that collagen and proteoglycan in these two tumours are well-defined and organized, likely contributing to resistance to macromolecular drug penetration. Enzymes such as collagenase have been used to digest these ECM proteins to improve penetration of nanoparticles.

Interstitial fluid pressure: The irregular vasculature in solid tumours consists of excessively-branched, dilated, leaky vessels with loosely-attached pericytes. Vessels are highly heterogenous in tumours and can cause variable blood flow to tumour tissues—this limits oxygen and nutrient access for cells, ultimately influencing metastasis and sensitivity to drug treatment to poorly perfused cells. Drugs diffuse down their concentration or pressure gradients, but as the network of vessels have high resistance to blood flow, drugs may instead be pulled into tissues via osmotic forces. Moreover, these irregularities in tumour tissues can be exacerbated by the lack of a proper lymphatic drainage, resulting in the accumulation of fluids and increased interstitial fluid pressure (IFP). IFP can vary between tumours of the same histological type, each then responding differently to chemotherapy or radiation. Normal interstitial pressure is close to atmospheric, the excess pressure ranging from 1-3 mm Hg. These incremental values can be elevated to up to 100 mm Hg in tumour tissues, which often correlates to poor survival and treatment responses in patients. IFP is also directly associated with tumour angiogenesis, whereby pro-angiogenic and anti-angiogenic factors are upregulated and downregulated in tumours, respectively. Blocking angiogenesis has shown to improve drug penetration against the pressure gradient. Angiogenesis can be downregulated by inhibiting vascular endothelial growth factor (VEGF), which is a family of secreted polypeptides that regulates blood vessel formation, or by blocking platelet-derived growth factor receptor (PDGF-R), which is a tyrosine-protein kinase that promotes blood vessel development when bound by a growth factor. PDGF-R is localized in the cells of mesenchymal origin, such as neurons and vascular smooth muscle cells, and is often overexpressed in tumour cells. Imatinib is a drug that binds to p-PDGFR-β and has been shown to downregulate VEGF, reduce IFP and increase tumour oxygenation. Similarly, the VEGF-specific antibody bevacizumab has shown to decrease microvascular density in five of six patients with rectal tumours.

Hypoxic core: Gradients of nutrients and oxygen levels are established in tumour tissues, with rapidly proliferating cells situated closer to the vasculature where levels are highest. In well-vascularized tissues, cells are localized within 50-100 p.m of perfused blood vessels; this distance can increase up to 200 μm for tumour cells, severely impairing blood flow and poor oxygen delivery to cells in tumour tissues. One study revealed that hypoxic (5 mmHg) and anoxic values are reached 70-80 p.m and 150 p.m away from the vessel, respectively. Hypoxia is associated with the malignant phenotypes of tumours, inducing genes that express angiogenic and metastatic factors. Extracellular acidity of pH 6.5-7.0 is also a common feature in areas distal from the vasculature in tumours and is a direct result of accumulated CO₂ and other metabolites produced from anaerobic glycolysis, such as lactic acid. In tumours, pH is shown to have dropped by a factor of 0.32, 10 to 100 p.m away from the vessel wall. Conventional anticancer agents have limited access to these cells due to irregular blood flow, poor rate of diffusion and binding to other tissue components; thus they are often detected at the rim of the tumour, if any at all. Furthermore, the cytotoxicity of chemotherapeutic drugs is reduced in hypoxic and acidic environments. Cytotoxicity of doxorubicin has been shown to be less substantial in low-pH-adapted cells than in normal cells, due to the transmembrane pH gradient that is established in these cells. Additionally, low pH has been shown to direct cells into the Gi phase, rendering them more resistant to the action of mioxantrone and topotecan. Secondary metabolites produced by glycolysis have also been targeted as a therapeutic strategy; this can be achieved by targeting lactate transporters, such as MCT1, resulting in the effective inhibition of hypoxia-inducible factor-1 (HIF-1)-dependent angiogenesis in endothelial cells.

Solid tumour therapy: Although surgical excision is the most widely used form of tumour therapy, it is most effective when the tumour is still considered small and confined to a limited area. This is, however, not an effective method for large, invasive and metastatic tumours, whereby resection of surrounding normal tissues can lead to organ dysfunction. Radiation is another commonly employed method for inoperable solid tumours, such as lung cancer, but the potential co-injury of normal tissues must be considered. This can result in toxic effects such as bone marrow suppression and inflammation of the surrounding tissues for the patient. Conventional chemotherapeutic agents function mostly by targeting actively-replicating malignant cells and are often combined with radiation and surgery to increase the effectiveness of treatment. However, this mechanism is also non-specific and often harms normal cells of the bone marrow, gastrointestinal tract, hair follicles and gonads. Therefore, specific targeting remains the desired goal since it would ensure increased effectiveness of anticancer agents, driving research into the development of alternative drug delivery systems and methods. New methods of payload delivery have been explored, such as nanoparticles, liposomes, and microspheres. These systems based on passive or active tumour targeting have been considered promising, but many are still limited by their larger size. Extravasation into the tumour interstitium remains a challenge, as liposomes of 100 nm have failed to extravasate into the SKOV-3 (human ovarian carcinoma) and B16BL6 (melanoma) tumours in mice. A better strategy for targeting is therefore needed.

Cell receptors: Tumour cells are equipped with cell-surface receptors, including/and not limited to HLA antigens, cytokine receptors and growth factor receptors. The overexpression of cellular receptors has long been exploited in targeting cancer therapeutics, especially the inhibition of growth factor receptors. A myriad of potential molecular targets have been identified in cancer therapeutics. A widely-known example is the HER-2/neu receptor, which can be inhibited by Herceptin®—an FDA-approved drug used for the treatment of patients with HER-2/neu-positive breast and gastric tumours. Currently marketed drugs include imatinib for myelogenous leukemia, cetuximab for EGFR-overexpressing metastatic colorectal cancer and bevacizumab for targeting VEGF of colorectal cancer. Certain key features of drug targets include: 1) they are critical in the perpetuation of cancer cells (not just implicated in the early stages of tumourigenesis); and 2) they are not critical in the function of normal cells. Moreover, they should be expressed at higher levels, if not exclusively, in the targeted tumour cells. Of the many growth factor receptors, receptor tyrosine kinases (RTKs) have been implicated in a wide range of cancers. ErbB family members are RTKs that are well-known targets in cancer therapy that have been implicated in the cancers of the breast, lung, colon, stomach, pancreatic, ovary, brain, prostate and kidney (Inchley, 1969). Small molecule tyrosine kinase inhibitors (TKIs) and monoclonal antibodies (mAbs) have been approved for use against ErbB family members.

Parameters affecting drug penetration into solid tumours: Physical properties of molecules such as particle size, shape, charge, and hydrophobicity can influence the passive transit through intercellular spaces of solid tumours. Delivery agents are also affected by contact with the ECM and cell surface. Numerous drug delivery technologies have been developed as carrier systems for therapeutically active agents, including liposomes, micelles, antibodies, affinity targeting and macromolecular drug carriers. However, these particles are found to be restricted to the tumour vasculature with very little infiltration due to their sizes. Adenoviral gene transfer in spheroids has also been assessed but poor penetration is observed despite the application of high titers of 10¹⁰ PFU/spheroid; transfection is observed only occurring in the first two cell layers of the spheroid.

All nanoparticles move more slowly through fluid than conventional therapeutic agents, as the process of diffusion is much more efficient for smaller particles. However, larger particles can benefit from the enhanced permeability and retention effect (EPR). The rate of clearance in tumour tissue is reduced due to the poor lymphatic system, which can facilitate the selective accumulation and retention of larger nanoparticles. Small molecules, however, can manage to escape the tumour vessels through the fenestrations. Dreher et al. studied the depth of penetration of dextrans with sizes ranging from 3.3 kDa to 2 MDa. After 30 min, maximal accumulation in the tumour is observed for dextrans 40-70 kDa (diameters of 11.2-14.6 nm), approximately 15 μm from the vessel wall. This is due to the increased accumulation of these molecules, whereas their lower molecular weight counterparts are subjected to a higher rate of clearance. Passive tumour targeting by nanoparticles relies primarily on EPR, which can enhance their accumulation in tumour tissues more than in healthy tissues.

Neutral and positive surface charges contribute to diffusion of nanoparticles into spheroids. Negatively-charged quantum dots are able to penetrate deeply into HeLa spheroids, contrary to their positively-charged counterparts that are confined to the rim. The shallow depth of penetration by cationic liposomes is suggested to have been caused by the electrostatic binding between the positive liposomes and negatively-charged surface of cells, inhibiting further diffusion. Surface coating, such as by PEG, can reduce these electrostatic interactions of nanoparticles with cells and assist in evasion from the reticuloendothelial system (RES). However, depth of penetration can be compromised due to the increase in overall size. Finally, the shape of the nanoparticle also determines its diffusivity. Although conventional carrier systems are spherical, rods or filamentous shapes may be more optimal. They have shown to diffuse more rapidly into spheroids with greater accumulation in vivo and in vitro. Nanorods and nanospheres with the same hydrodynamic diameter of 33 nm are compared for transport across porous membranes in vitro. Nanorods are able to diffuse 5.3 times deeper than nanospheres through 100-400 nm collagen gel pores, which are approximate pore sizes of tumour vascular walls.

Fusion of molecules to enhance cellular uptake and penetration has been widely investigated. These molecules include tumour-homing and cell-penetrating peptides, such as arginine-glycine-aspartic acid (RGD), TAT, and MPG. The fusion of RGD to lipid coated nanoparticles significantly improved its biodistribution in spheroids of C6 glioblastoma cells that overexpress integrin alphvbeta3 in vitro. iRGD is a 9-amino acid, cyclic tumour-penetrating peptide with greater binding affinity than the standard RGD motif. The co-administration of iRGD significantly improved the extravasation of nanoparticles from the tumour vessels into the glioma parenchyma, resulting in overall greater accumulation in nude mice. Another group designed liposomal systems modified with TAT and T7 to enhance penetration into the glioma the blood-brain barrier, respectively. TAT-liposomes showed deeper penetration into the C6 tumour spheroids in vitro than T7-liposomes, but the opposite is observed in vivo. This is probably caused by the nonselectivity of TAT, as well as interference of natural diffusivity by the liposomes. Having both molecules fused to liposomes resulted in the highest accumulation in the glioma due to synergistic effects. The ability of nanoparticles to diffuse through the spheroid does not always correlate to their total accumulation in the spheroid. The conjugation of MPG, synthetic peptide derived from HIV gp41 and SV40, increased uptake of nanoparticles in HeLa spheroids by almost three-fold. PEG-nanoparticles, however, are localized mainly in the spheroid periphery, but are able to penetrate tissue culture by almost two-folds further than the MPG-nanoparticles.

### SUMMARY

Solid tumours not only comprise a heterogeneous population of cancer cells and stromal cells, but have a complex tumour microenvironment comprising host cells and molecules, secreted factors, blood vessels and extracellular matrix proteins as well as many other components. The inventors have shown that recombinant bacteriophage presenting an EGFR-binding moiety, EGF, on their cell surface are able to target and penetrate multicellular spheroids generated from colorectal adenocarcinoma HT29 cells. These multicellular spheroids mimic the in vivo tumour microenvironment and demonstrate similar responses to therapies as tumours in vivo. The display of EGF on lambda phage enhances binding, uptake and accumulation in the tumour microenvironment of the spheroids, permits the internalization of bacteriophage within cells present in the tumour, and ultimately slows tumour formation and growth.

The present disclosure includes modified bacteriophage, compositions comprising a plurality of modified bacteriophage, methods and uses thereof, kits and commercial packages.

In one aspect, the disclosed subject matter includes a pharmaceutical composition comprising a plurality of bacteriophage engineered to present an epidermal growth factor receptor (EGFR)-binding moiety on the bacteriophage cell surface. The EGFR-binding moiety is capable of binding the extracellular domain of the EGFR and may possess one or more of the following functional properties including: inhibiting dimerization of an EGFR to an Erb family member; competing with EGF for binding of an EGFR; modulating one or more EGFR downstream signaling pathways; promoting internalization of an EGFR into a cell; or a combination thereof.

In one aspect, there is provided a bacteriophage engineered to present an epidermal growth factor receptor (EGFR)-binding moiety on the bacteriophage cell surface, wherein the EGFR-binding moiety is for, or is capable of, binding the extracellular domain of an EGFR.

In another aspect, there is provided a pharmaceutical composition for use in a method of treating an EGFR-positive tumour in a subject in need thereof.

In another aspect, there is provided a method of treating an EGFR-positive tumour in a subject in need thereof.

The method of treating an EGFR-positive tumour comprises administering to the subject a plurality of bacteriophage engineered to present an epidermal growth factor receptor (EGFR)-binding moiety on the bacteriophage cell surface, the EGFR-binding moiety is for, or is capable of binding the extracellular domain of an EGFR, in a dose effective to treat the tumor. The plurality of bacteriophage may provided with a pharmaceutically acceptable excipient, diluent or carrier to form a pharmaceutical composition.

In an embodiment, the bacteriophage is a lytic bacteriophage. In an embodiment, the bacteriophage is a λ bacteriophage T7 or T4 bacteriophage. In an embodiment, the bacteriophage is λF7. In an embodiment, the EGFR-binding moiety is an EGFR ligand. In an embodiment, the EGFR ligand is an EGF or a functional derivative thereof. In an embodiment, a titration of the bacteriophage in the composition ranges from 10⁷ PFU/ml to 10¹⁰ PFU/ml.

In an embodiment the tumour is a solid tumour and/or a spheroid. In some embodiments, the dose is effective to increase the penetration of the plurality of bacteriophage into the tumour. Alternatively and/or additionally, the dose is effective to increase an internalization of the plurality of bacteriophage into tumour cells and cells of the tumour microenvironment. Alternatively and/or additionally, the dose is effective to increase accumulation of the plurality of bacteriophage in the tumour. Further, it is also preferred that the dose is effective to reduce a growth of the tumour.

In some embodiments, the tumour has a genetic signature comprising wildtype KRAS and mutated BRAF.

Other aspects and features of the present disclosure will become apparent to those ordinarily skilled in the art upon review of the following description of specific embodiments in conjunction with the accompanying figures.

### BRIEF DESCRIPTION OF THE DRAWINGS

A better understanding of the features and advantages of the present invention will be obtained by reference to the following detailed description that sets forth illustrative embodiments, in which the principles of the invention are utilized. Embodiments of the present disclosure will now be described, by way of example only, with reference to the attached Figures.

FIG. 1 shows schematic diagram of receptor tyrosine kinase (RTK);

FIG. 2 shows a schematic diagram of a multicellular spheroid;

FIG. 3 shows a schematic diagram of bacteriophage λ and one face of the capsid;

FIG. 4 shows data of an internalization assay;

FIG. 5 shows growth of HT29 spheroids at various seeding concentrations;

FIG. 6 shows growth of A549 spheroids at various seeding concentrations;

FIG. 7 shows growth of MDA-MB-231 spheroids at various seeding concentrations;

FIG. 8 shows growth of A2780 spheroids at various seeding concentrations;

FIG. 9 shows HT29 cells seeded at various concentrations after 7 d;

FIG. 10 shows optical cross-sections of NIH3T3 spheroids treated with fluorescent λ. phages;

FIG. 11 shows NIH3T3 spheroid treated with polyethylene glycol;

FIG. 12 shows optical cross-sections of HT29 spheroids treated with wildtype phages and EGF-displaying phages for 30 min (top) and 4 h (bottom);

FIG. 13 shows representative cross-sections of HT29 spheroids treated with wildtype phages and EGF-displaying phages for 8 h (top) and 24 h (bottom);

FIG. 14A shows collective Z-stacks of HT29 spheroids treated with EGF-displaying phages for 24 h;

FIG. 14B shows collective Z-stacks of HT29 spheroids treated with wildtype phages for 24 h;

FIG. 15 shows phage-associated fluorescence emitted inter- and intracellularly of HT29 cells;

FIG. 16 shows testing the action of ammonium chloride as a lysosomotropic agent to improve phage recovery;

FIG. 17 shows percent internalization of phages determined from HT29 spheroids;

FIG. 18 shows effects of D::EGF phages and wildtype phages on the growth of HT29 spheroids;

FIG. 19 shows HT29 cells co-seeded with D::EGF phages or wildtype phages;

FIG. 20 shows growth of pre-chilled HT29 cells with phages;

FIG. 21 shows Effects of wildtype or Hi-D::EGF phages on HT29 spheroids grown for 5 d;

FIG. 22 shows HT29 cell proliferation (MTT) assay in response to phage treatment relative to control;

FIG. 23 shows HT29 spheroid cell viabilities relative to control in response to phage treatment; and

FIG. 24 shows a schematic diagram of a VEGF-linker-gpD fusion.

### DETAILED DESCRIPTION

As used in the specification and appended claims, unless specified to the contrary, the following terms have the meaning indicated below.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of or “consisting of.” The phrase “consisting essentially of is used herein to require the specified feature(s) as well as those which do not materially affect the character or function of the claimed disclosure. As used herein, the term “consisting” is used to indicate the presence of the recited feature alone.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of can include determining the amount of something present in addition to determining whether it is present or absent depending on the context.

As used herein, “treat”, “treating”, “treatment of”, are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease or condition, or to a patient reporting one or more of the physiological symptoms of a disease, even though a diagnosis of this disease has not been made. These terms include the eradication, removal, amelioration, modification, reduction, management or control of a tumour, tumour cells or cancer. These terms also encompass ameliorating one or more pathological aspects of cancer and can comprise reducing tumour growth, inhibiting metastasis, reducing tumour burden, extending progression free survival, or extending overall life span.

The terms “therapeutically effective amount”, “effective amount” or “sufficient amount” mean a quantity sufficient to achieve a desired result, for example an amount effective to reduce tumour growth, when administered to a subject, including a mammal, for example a human. Therapeutically effective amounts may vary according to factors such as the age, sex, and weight of the subject. Dosage or treatment regimens may be adjusted to provide the optimum therapeutic response, as is understood by a skilled person. For example, administration of a therapeutically effective amount of the bacteriophage described herein is, in aspects, sufficient to reduce tumour growth of an epidermal growth factor receptor (EGFR)-positive tumour.

The term “cancer” (e.g. neoplastic disorder) as used herein refers to a disorder involving aberrant cell growth, proliferation or division (e.g. neoplasia). As neoplastic cells grow and divide they pass on their genetic mutations and proliferative characteristics to progeny cells. A “tumour” (e.g. neoplasm) is an accumulation of neoplastic cells. The methods and combinations disclosed herein may be used in the treatment of cancer, neoplastic cells, tumors and/or symptoms associated therewith.

By “inhibiting” or “reducing”, e.g. tumour growth, it is generally meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are either not treated or are not subjected to the methods and combinations of the present application. By “reducing” a tumor it is generally meant to reduce the size of a tumour, as measured using methods known to those of ordinary skill in the art, by, for example, 10%, 20%, 30%, 40%, 50%, 60%, 70%, 80%, 90%, 95%, or 100%, when compared to tumour size before treatment or compared to tumors that are not subjected to the methods and combinations of the present application.

An “EGFR-positive tumour” is a tumour in which EGFR is up-regulated, i.e. EGFR is expressed on the cell surface in at least a portion of the cells of the tumour. A person of skill in the art would know how to test if a tumour expresses EGFR using assays that are standard in the art including, for example, immunohistochemistry (IHC) and/or Fluorescence In Situ Hybridization FISH/SISH/ISH assays. The EGFR receptor may be wildtype or may be mutated.

An EGFR-binding moiety is a moiety that is able to bind the EGFR, particularly the extracellular domain of the EGFR. By binding the EGFR extracellular domain, the EGFR-binding moiety targets the bacteriophage to an EGFR-positive tumour. In addition, an EGFR-binding moiety has one or more of the following functional properties. The EGFR-binding moiety may be capable of inhibiting dimerization of an EGFR to an Erb family member, including, for example EGFR, ErbB2, ErbB3 or ErbB4. The EGFR-binding moiety may compete with EGF for binding of the EGFR. The EGFR-binding moiety may be capable of modulating one or more EGFR downstream signaling pathways. The EGFR-binding moiety may activate EGFR downstream signaling pathways or may inhibit EGFR downstream signaling pathways. EGFR downstream signaling pathways include, for example, Ras/MAPK, PLCγ1/PKC PI3K/Akt and STAT pathways, which ultimately alter gene expression controlling cell properties, including for example cell growth and survival. EGFR downstream signaling pathways are well characterized and the person of skill in the art would be aware of many standard assays to test whether an EGFR-binding moiety is able to modulate signaling upon binding of the EGFR-binding moiety to the EGFR. The EGFR-binding moiety may also be capable of promoting internalization of an EGFR into a cell upon binding the EGFR. Internalization assays are well known in the art. The EGFR-binding moiety may be a part of a molecule (for example, EGF in a capsid protein::EGF fusion protein).

The terms “subject,” “individual,” or “patient” are often used interchangeably herein. A “subject” can be a biological entity containing expressed genetic materials. The biological entity can be a plant, animal, or microorganism, including, for example, bacteria, viruses, bacteriophage, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

A “pharmaceutical composition” refers to a combination of ingredients that facilitates administration of one or more agents of interest (e.g. a therapeutic agent or a co-administrable agent) to an organism, e.g. a human or animal. A pharmaceutical composition generally comprises one or more agents of interest together with one or more pharmaceutically acceptable carriers or excipients. Many pharmaceutically-acceptable “carriers” or “excipients” are known in the art and these generally refers to a pharmaceutically-acceptable materials, compositions, or vehicles, including liquid or solid fillers, diluents, excipients, solvents, binders, or encapsulating materials. Each component in the composition must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams &amp; Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. A skilled person would readily appreciate there are many ways to encode the above-described recombinant polypeptide (e.g. due to degeneracy of the genetic code), all of which are encompassed. Deletions, insertions, and substitutions may also be permitted if protein function remains substantially intact. For instance, nucleic acids may have 75% or greater, 80% or greater, 85% or greater, 90% or greater, 95% or greater, or 99% or greater identity to wildtype or reference sequences may be encompassed.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence.

Percent (%) sequence identity with respect to a reference nucleotide or polypeptide sequence is the percentage of nucleotides or amino acid residues in a candidate sequence that are identical with the nucleotides or amino acid residues in the reference nucleotide or polypeptide sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code.

As used herein the terms “lytic bacteriophage” or “lytic phage” are used interchangeably and refer to a bacteriophage having a lytic life cycle. Lytic phages will lyse a host cell in order to release new phage particles. Lytic bacteriophage can be used in a method of lytic phage display in order to generate highly decorated phage particles.

As used herein, the term “isolated” means altered or removed the natural state.

As used herein, the term “about” a number refers to that number plus or minus 10% of that number. The term “about” a range refers to that range minus 10% of its lowest value and plus 10% of its greatest value.

TABLE 1
Abbreviations

| | |
|---|---|
| 5-FU | Fluorouracil |
| A2780 | Human ovarian cancer cell line |
| A549 | Adenocarcinomic human alveolar basal epithelial cells |
| AAVP | Adeno-associated virus/phage |
| ANOVA | Analysis of variation |
| ATCC | American Type Culture Collection |
| ATP | Adenosine triphosphate |
| B16 | Mouse melanoma cells |
| BB4 | Suppressor E. coli strain (supE supF hdR) that is a high efficiency |
| | plating natural host for bacteriophage λ and amber mutant |
| | derivatives |
| BRAF | Gene that encodes proto-oncogene B-Raf, involved in sending |
| | signals inside cells involved in directing cell growth |
| BSA | Bovine serum albumin |
| C6 | Rat glioma cells |
| CI | λ repressor gene product, allows phage to reside in lysogenic state |
| c1857 | A common temperature-sensitive allele of the λ cl repressor |
| COS-I | Fibroblast-like cells derived from monkey kidney tissue |
| D | Gene encoding gene product gpD |
| gpD::EGF | EGF peptide fused onto the gpD capsid protein expressed by a |
| | suppressor host carrying the D::EGF fusion plasmid. |
| DMEM | Dulbecco's Modified Eagle's Medium |
| DMSO | Dimethyl sulfoxide |
| ECM | Extracellular matrix |
| EDTA | Ethylenediaminetetraacetic acid, a chelating agent that possesses the |
| | ability to sequester divalent metal cations such as Mg²⁺ |
| EGF | Epidermal growth factor |
| eGFP | Gene encoding enhanced green fluorescent protein |
| EGFR | Epidermal growth factor receptor |
| EOP | Efficiency of plating, the relative viability of phage sample in |
| | comparison to a positive control |
| EPR | Enhanced permeability and retention effect |
| ErbB | Family of proteins containing four receptor tyrosine kinases |
| Erk | Extracellular signal-regulated kinases |
| FACS | Fluorescence-activated cell sorter |
| FBS | Foetal bovine serum |
| FGF2 | Fibroblast growth factor |
| G₁ | Gap I phase, first of four phases in the cell cycle in eukaryotic cell |
| | division |
| GAGs | Glycosaminoglycans, long branched polysaccharides consisting of |
| | amino sugar with uronic sugar or galactose. |
| GI | Gastrointestinal |
| gp23 | Major capsid protein of T4 phage |
| gp24 | Bacteriophage T4 capsid vertex protein gp24 |
| gp41 | Glycoprotein 41 is a subunit of the envelope protein complex of |
| | retroviruses, including human immunodeficiency virus (HIV) |
| gpD | λ gene product D, a major structural protein in the λ phage capsid |
| | head |
| gpDQ68S | Allele conferred from growing λF7 on amber suppressor strain |
| | W3101 SupD (serU132) which inserts amino acid serine in place of |
| | the amber stop codon |
| gpDQ68Y | Allele conferred from growing XF7 on amber suppressor strain |
| | W3101 SupF (tyrT5888) which inserts amino acid tyrosine in place |
| | of the amber stop codon |
| gpE | λ gene E product - major structural protein in the λ phage capsid |
| gpV | Tail protein of phage λ |
| Grb2 | Growth factor receptor-bound protein 2 |
| HCT-116 | Human colon cancer cell line |
| HER-2/neu | Human epidermal growth factor receptor 2 |
| Hi-D::EGF | λF7 phage with maximal EGF surface decoration. Generated using |
| phage | SupD E. coli host at 37° C. |
| HIF-1 | Hypoxia inducible factor-I |
| HLA | Human leukocyte antigen |
| HPEVI | Human parechovirus 1 |
| HT29 | Human colon adenocarcinoma cell line |
| IF λ | Interferon λ |
| IFP | Interstitial fluid pressure |
| IgG | Immunoglobulin G antibody isotype |
| IL | Interleukin |
| IL 1RN | Anti-inflammatory ILI receptor antagonist |
| KGD | Lysine-Glycine-Aspartic acid |
| KRAS | Gene that encodes K-Ras that is part of the RAS/MAPK pathway |
| LB | Luria Bertani broth, a widely used rich culture medium, for growth |
| | of bacteria and phage |
| Lo-D::EGF | λF7 phages with 40% less surface decoration than Hi-D::EGF |
| phage | phages. Generated using SupF E. coli host at 37° C. |
| LOX | Lysyl oxidase |
| M13 | A filamentous ssDNA bacteriophage |
| mAbs | Monoclonal antibodies |
| MAPK | Mitogen-activated protein kinases |
| MCTS | Multicellular tumour spheroids |
| MDA-MB-231 | aggressive, invasive human breast cancer cell line |
| MEK | Mitogen-activated protein kinase kinase |
| MPG | Cell penetrating peptide, short amphipathic peptides forming stable |
| | nanoparticles with nucleic acids |
| MTT | 3-(4,5-dimethylthiazol-2-yl-2,5-diphenyltetrazolium bromide |
| NilBT3 | Mouse fibroblast cells |
| OSF-2 | Osteoblast-specific factor 2 |
| PBMC | Peripheral blood mononuclear cells |
| PBS | Phosphate buffered saline |
| pD | Plasmid-borne D, resultant of the complementation of Dam15 from a |
| | plasmid |
| PEG | Polyethylene glycol |
| PFU | Plaque forming unit-single immobilized phage that grows to form a |
| | plaque on an agar cell lawn. A means of calculating lysate titer |
| PI(3)K | Phosphoinositide 3-kinase |
| PKC | Protein kinase C |
| p-PDGFR-β | Platelet-derived growth factor receptor beta protein encoded by the |
| | PDGFRB gene |
| pPL451 | High copy plasmid vector under the control of temperature sensitive |
| | repressor, CI857 |
| PTPIB | Protein tyrosine phosphatase |
| RES | Reticuloendothelial system |
| RGD | Arginine-glycine-aspartic acid |
| RTK | Receptor tyrosine kinase |
| SDS | Sodium dodecyl sulfate |
| serU132 | Mutation encoding amber suppressor strain W3101 SupD |
| Soc | Major coat protein of T4 phage, present in ~870 copies |
| SOCS3 | Suppressor of cytokine signaling 3 |
| Src | Proto-oncogene tyrosine-protein kinase |
| STAT | Signal transducer and activator of transcription |
| Sup−/sup | A wild type E. coli strain that does not confer any suppressor activity |
| Sup+/sup+ | A mutant E. coli strain that confers suppressor activity to conditional |
| | mutations |
| SupD/supD | A tRNA mutation in E. coli that suppresses the amber stop codon |
| | conditional mutation (UAG) by reading this codon and inserting |
| | instead a serine amino acid |
| SupF/supF | A tRNA mutation of E. coli that suppresses the amber stop codon by |
| | inserting instead a tyrosine amino acid at this codon |
| SV40 | Simian virus 40, a polyomavirus that is found in both monkeys and |
| | humans |
| T4 | Member of the lytic T-even myoviridae bacteriophage of E. coli that |
| | is only capable of undergoing a lytic lifecycle |
| T7 | Member of the type T podoviridae bacteriophage family that infects |
| | E. coli. This phage is only capable of undergoing a lytic lifecycle |
| TAT | Gene product of the tat gene, the “trans-activator of transcription”, |
| | from HIV-I which acts as a regulatory protein by dramatically |
| | enhancing endocytosis and the efficiency of viral transcription |
| TGF-α | Transforming growth factor-α |
| TKI | Tyrosine kinase inhibitors |
| TNF-α | Tumour necrosis factor-α |
| tRNA | Transfer RNA, physical link between the nucleotide sequence of |
| | nucleic acids (DNA/RNA) and the amino acid sequence of proteins |
| tyrT5888 | Mutation encoding amber suppressor strain W3101 SupF |
| UAG | Amber stop codon |
| VEGF | Vascular endothelial growth factor |
| W3101 | E. coli strain (F-, galT22, λ-, IN(rrnD-rrnE)1, rph-1) that is a high |
| | efficiency plating natural host for bacteriophage λ |
| WT phages | λF7 phages without any surface decoration |
| λDam | λphage that contains an amber mutation within the gene for protein |
| | gpD and thus can only produce wild-type length gpD alleles within |
| | specialized strains of E. coli |
| λF7 | Synonymous with λDam15Dam15imm21cl[Tts] that possesses a |
| | defective (suppressible) D gene and the immunity region (imm) of |
| | phage 21 |

TABLE 1A
Table of Sequences

| | | |
|---|---|---|
| SEQ ID | Human EGF | MLLTLIILLPVVSKFSFVSLSAPQHWSCPEGTLAGNGNSTCV |
| NO: 1 | amino acid | GPAPFLIFSHGNSIFRIDTEGTNYEQLVVDAGVSVIMDFHYN |
| | sequence | EKRIYWVDLERQLLQRVFLNGSRQERVCNIEKNVSGMAIN |
| | | WINEEVIWSNQQEGIITVTDMKGNNSHILLSALKYPANVAVD |
| | | PVERFIFWSSEVAGSLYRADLDGVGVKALLETSEKITAVSLD |
| | | VLDKRLFWIQYNREGSNSLICSCDYDGGSVHISKHPTQHNL |
| | | FAMSLFGDRIFYSTWKMKTIWIANKHTGKDMVRINLHSSFV |
| | | PLGELKVVHPLAQPKAEDDTWEPEQKLCKLRKGNCSSTVC |
| | | GQDLQSHLCMCAEGYALSRDRKYCEDVNECAFWNHGCTL |
| | | GCKNTPGSYYCTCPVGFVLLPDGKRCHQLVSCPRNVSEC |
| | | SHDCVLTSEGPLCFCPEGSVLERDGKTCSGCSSPDNGGC |
| | | SQLCVPLSPVSWECDCFPGYDLQLDEKSCAASGPQPFLLF |
| | | ANSQDIRHMHFDGTDYGTLLSQQMGMVYALDHDPVENKIY |
| | | FAHTALKWIERANMDGSQRERLIEEGVDVPEGLAVDWIG |
| | | RRFYWTDRGKSLIGRSDLNGKRSKIITKENISQPRGIAVHPM |
| | | AKRLFWTDTGINPRIESSSLQGLGRLVIASSDLIWPSGITIDF |
| | | LTDKLYWCDAKQSVIEMANLDGSKRRRLTQNVGHPFAVAV |
| | | FEDYVWFSDWAMPSVIRVNKRTGKDRVRLQGSMLKPSSL |
| | | VVVHPLAKPGADPCLYQNGGCEHICKKRLGTAWCSCREG |
| | | FMKASDGKTCLALDGHQLLAGGEVDLKNQVTPLDILSKTRV |
| | | SEDNITESQHMLVAEIMVSDQDDCAPVGCSMYARCISEGE |
| | | DATCQCLKGFAGDGKLCSDIDECEMGVPVCPPASSKCINT |
| | | EGGYVCRCSEGYQGDGIHCLDIDECQLGVHSCGENASCT |
| | | NTEGGYTCMCAGRLSEPGLICPDSTPPPHLREDDHHYSVR |
| | | NSDSECPLSHDGYCLHDGVCMYIEALDKYACNCVVGYIGE |
| | | RCQYRDLKWWELRHAGHGQQQKVIVVAVCVVVLVMLLLLS |
| | | LWGAHYYRTQKLLSKNPKNPYEESSRDVRSRRPADTED |
| | | GMSSCPQPWFVVIKEHQDLKNGGQPVAGEDGQAADGSM |
| | | QPTSWRQEPQLCGMGTEQGCWIPVSSDKGSCPQVMERS |
| | | FHMPSYGTQTLEGGVEKPHSLLSANPLWQQRALDPPHQM |
| | | ELTQ |
| SEQ ID | Capsid | MTSKETFTHY QPQGNSDPAH TATAPGGLSA |
| NO: 2 | decoration | KAPAMTPLML DTSSRKLVAW DGTTDGAAVG |
| | protein | ILAVAADQTS TTLTFYKSGT FRYEDVLWPE |
| | Gene D | AASDETKKRT AFAGTAISIV |
| | Bacteriophage | |
| | lambda gpD | |
| | capsid protein, | |
| | amino acid | |
| | sequence | |
| SEQ ID | Linker sequence | TSGSGSGSGSGT |
| NO: 3 | (protein) | |
| SEQ ID | Human EGF, | CAGCCGCATCTGGGGTCAATCATACTCACCTTGCCCGGGCC |
| NO: 4 | nucleotide | ATGCTCCAGCAAAATCAAGCTGTTTTCTTTTGAAAGTTCAAAC |
| | sequence | TCATCAAGATTATGCTGCTCACTCTTATCATTCTGTTGCCAGT |
| | | AGTTTCAAAATTTAGTTTTGTTAGTCTCTCAGCACCGCAGCAC |
| | | TGGAGCTGTCCTGAAGGTACTCTCGCAGGAAATGGGAATTC |
| | | TACTTGTGTGGGTCCTGCACCCTTCTTAATTTTCTCCCATGGA |
| | | AATAGTATCTTTAGGATTGACACAGAAGGAACCAATTATGAG |
| | | CAATTGGTGGTGGATGCTGGTGTCTCAGTGATCATGGATTTT |
| | | CATTATAATGAGAAAAGAATCTATTGGGTGGATTTAGAAAGAC |
| | | AACTTTTGCAAAGAGTTTTTCTGAATGGGTCAAGGCAAGAGA |
| | | GAGTATGTAATATAGAGAAAAATGTTTCTGGAATGGCAATAAA |
| | | TTGGATAAATGAAGAAGTTATTTGGTCAAATCAACAGGAAGG |
| | | AATCATTACAGTAACAGATATGAAAGGAAATAATTCCCACATT |
| | | CTTTTAAGTGCTTTAAAATATCCTGCAAATGTAGCAGTTGATC |
| | | CAGTAGAAAGGTTTATATTTTGGTCTTCAGAGGTGGCTGGAA |
| | | GCCTTTATAGAGCAGATCTCGATGGTGTGGGAGTGAAGGCT |
| | | CTGTTGGAGACATCAGAGAAAATAACAGCTGTGTCATTGGAT |
| | | GTGCTTGATAAGCGGCTGTTTTGGATTCAGTACAACAGAGAA |
| | | GGAAGCAATTCTCTTATTTGCTCCTGTGATTATGATGGAGGTT |
| | | CTGTCCACATTAGTAAACATCCAACACAGCATAATTTGTTTGC |
| | | AATGTCCCTTTTTGGTGACCGTATCTTCTATTCAACATGGAAA |
| | | ATGAAGACAATTTGGATAGCCAACAAACACACTGGAAAGGAC |
| | | ATGGTTAGAATTAACCTCCATTCATCATTTGTACCACTTGGTG |
| | | AACTGAAAGTAGTGCATCCACTTGCACAACCCAAGGCAGAA |
| | | GATGACACTTGGGAGCCTGAGCAGAAACTTTGCAAATTGAG |
| | | GAAAGGAAACTGCAGCAGCACTGTGTGTGGGCAAGACCTCC |
| | | AGTCACACTTGTGCATGTGTGCAGAGGGATACGCCCTAAGT |
| | | CGAGACCGGAAGTACTGTGAAGATGTTAATGAATGTGCTTTT |
| | | TGGAATCATGGCTGTACTCTTGGGTGTAAAAACACCCCTGGA |
| | | TCCTATTACTGCACGTGCCCTGTAGGATTTGTTCTGCTTCCT |
| | | GATGGGAAACGATGTCATCAACTTGTTTCCTGTCCACGCAAT |
| | | GTGTCTGAATGCAGCCATGACTGTGTTCTGACATCAGAAGGT |
| | | CCCTTATGTTTCTGTCCTGAAGGCTCAGTGCTTGAGAGAGAT |
| | | GGGAAAACATGTAGCGGTTGTTCCTCACCCGATAATGGTGG |
| | | ATGTAGCCAGCTCTGCGTTCCTCTTAGCCCAGTATCCTGGGA |
| | | ATGTGATTGCTTTCCTGGGTATGACCTACAACTGGATGAAAA |
| | | AAGCTGTGCAGCTTCAGGACCACAACCATTTTTGCTGTTTGC |
| | | CAATTCTCAAGATATTCGACACATGCATTTTGATGGAACAGAC |
| | | TATGGAACTCTGCTCAGCCAGCAGATGGGAATGGTTTATGCC |
| | | CTAGATCATGACCCTGTGGAAAATAAGATATACTTTGCCCATA |
| | | CAGCCCTGAAGTGGATAGAGAGAGCTAATATGGATGGTTCC |
| | | CAGCGAGAAAGGCTTATTGAGGAAGGAGTAGATGTGCCAGA |
| | | AGGTCTTGCTGTGGACTGGATTGGCCGTAGATTCTATTGGAC |
| | | AGACAGAGGGAAATCTCTGATTGGAAGGAGTGATTTAAATGG |
| | | GAAACGTTCCAAAATAATCACTAAGGAGAACATCTCTCAACC |
| | | ACGAGGAATTGCTGTTCATCCAATGGCCAAGAGATTATTCTG |
| | | GACTGATACAGGGATTAATCCACGAATTGAAAGTTCTTCCCT |
| | | CCAAGGCCTTGGCCGTCTGGTTATAGCCAGCTCTGATCTAAT |
| | | CTGGCCCAGTGGAATAACGATTGACTTCTTAACTGACAAGTT |
| | | GTACTGGTGCGATGCCAAGCAGTCTGTGATTGAAATGGCCA |
| | | ATCTGGATGGTTCAAAACGCCGAAGACTTACCCAGAATGATG |
| | | TAGGTCACCCATTTGCTGTAGCAGTGTTTGAGGATTATGTGT |
| | | GGTTCTCAGATTGGGCTATGCCATCAGTAATAAGAGTAAACA |
| | | AGAGGACTGGCAAAGATAGAGTACGTCTCCAAGGCAGCATG |
| | | CTGAAGCCCTCATCACTGGTTGTGGTTCATCCATTGGCAAAA |
| | | CCAGGAGCAGATCCCTGCTTATATCAAAACGGAGGCTGTGA |
| | | ACATATTTGCAAAAAGAGGCTTGGAACTGCTTGGTGTTCGTG |
| | | TCGTGAAGGTTTTATGAAAGCCTCAGATGGGAAAACGTGTCT |
| | | GGCTCTGGATGGTCATCAGCTGTTGGCAGGTGGTGAAGTTG |
| | | ATCTAAAGAACCAAGTAACACCATTGGACATCTTGTCCAAGA |
| | | CTAGAGTGTCAGAAGATAACATTACAGAATCTCAACACATGC |
| | | TAGTGGCTGAAATCATGGTGTCAGATCAAGATGACTGTGCTC |
| | | CTGTGGGATGCAGCATGTATGCTCGGTGTATTTCAGAGGGA |
| | | GAGGATGCCACATGTCAGTGTTTGAAAGGATTTGCTGGGGA |
| | | TGGAAAACTATGTTCTGATATAGATGAATGTGAGATGGGTGT |
| | | CCCAGTGTGCCCCCCTGCCTCCTCCAAGTGCATCAACACCG |
| | | AAGGTGGTTATGTCTGCCGGTGCTCAGAAGGCTACCAAGGA |
| | | GATGGGATTCACTGTCTTGATATTGATGAGTGCCAACTGGGG |
| | | GTGCACAGCTGTGGAGAGAATGCCAGCTGCACAAATACAGA |
| | | GGGAGGCTATACCTGCATGTGTGCTGGACGCCTGTCTGAAC |
| | | CAGGACTGATTTGCCCTGACTCTACTCCACCCCCTCACCTCA |
| | | GGGAAGATGACCACCACTATTCCGTAAGAAATAGTGACTCTG |
| | | AATGTCCCCTGTCCCACGATGGGTACTGCCTCCATGATGGT |
| | | GTGTGCATGTATATTGAAGCATTGGACAAGTATGCATGCAAC |
| | | TGTGTTGTTGGCTACATCGGGGAGCGATGTCAGTACCGAGA |
| | | CCTGAAGTGGTGGGAACTGCGCCACGCTGGCCACGGGCAG |
| | | CAGCAGAAGGTCATCGTGGTGGCTGTCTGCGTGGTGGTGCT |
| | | TGTCATGCTGCTCCTCCTGAGCCTGTGGGGGGCCCACTACT |
| | | ACAGGACTCAGAAGCTGCTATCGAAAAACCCAAAGAATCCTT |
| | | ATGAGGAGTCGAGCAGAGATGTGAGGAGTCGCAGGCCTGCT |
| | | GACACTGAGGATGGGATGTCCTCTTGCCCTCAACCTTGG |
| | | GTGGTTATAAAAGAACACCAAGACCTCAAGAATGGGGGTCAA |
| | | CCAGTGGCTGGTGAGGATGGCCAGGCAGCAGATGGGTCAA |
| | | TGCAACCAACTTCATGGAGGCAGGAGCCCCAGTTATGTGGA |
| | | ATGGGCACAGAGCAAGGCTGCTGGATTCCAGTATCCAGTGA |
| | | TAAGGGCTCCTGTCCCCAGGTAATGGAGCGAAGCTTTCATAT |
| | | GCCCTCCTATGGGACACAGACCCTTGAAGGGGGTGTCGAGA |
| | | AGCCCCATTCTCTCCTATCAGCTAACCCATTATGGCAACAAA |
| | | GGGCCCTGGACCCACCACACCAAATGGAGCTGACTCAGTGA |
| | | AAACTGGAATTAAAAGGAAAGTCAAGAAGAATGAACTATG |
| SEQ ID | gpD nucleotide | ATGACGAGCAAAGAAACCTTTACCCATTACCAGCCGCAG |
| NO: 5 | sequence | GGCAACAGTGACCCGGCTCATACCGCAACCGCGCCCGG |
| | | CGGATTGAGTGCGAAAGCGCCTGCAATGACCCCGCTGA |
| | | TGCTGGACACCTCCAGCCGTAAGCTGGTTGCGTGGGAT |
| | | GGCACCACCGACGGTGCTGCCGTTGGCATTCTTGCGGT |
| | | TGCTGCTGACCAGACCAGCACCACGCTGACGTTCTACAA |
| | | GTCCGGCACGTTCCGTTATGAGGATGTGCTCTGGCCGG |
| | | AGGCTGCCAGCGACGAGACGAAAAAACGGACCGCGTTT |
| | | GCCGGAACGGCAATCAGCATCGTT |
| SEQ ID | Linker, nucleotide | ACTAGCGGTTCCGGTTCTGGTTCCGGTTCTGGTTCCGGT |
| NO: 6 | sequence | TCTGGCGGTACC |
| SEQ ID | gpD-linker-EGF | ATGACGAGCAAAGAAACCTTTACCCATTACCAGCCGCAG |
| NO: 7 | nucleotide | GGCAACAGTGACCCGGCTCATACCGCAACCGCGCCCGG |
| | sequence | CGGATTGAGTGCGAAAGCGCCTGCAATGACCCCGCTGA |
| | | TGCTGGACACCTCCAGCCGTAAGCTGGTTGCGTGGGAT |
| | | GGCACCACCGACGGTGCTGCCGTTGGCATTCTTGCGGT |
| | | TGCTGCTGACCAGACCAGCACCACGCTGACGTTCTACAA |
| | | GTCCGGCACGTTCCGTTATGAGGATGTGCTCTGGCCGG |
| | | AGGCTGCCAGCGACGAGACGAAAAAACGGACCGCGTTT |
| | | GCCGGAACGGCAATCAGCATCGTTACTAGCGGTTCCGG |
| | | TTCTGGTTCCGGTTCTGGTTCCGGTTCTGGCGGTACCCA |
| | | GCCGCATCTGGGGTCAATCATACTCACCTTGCCCGGGC |
| | | CATGCTCCAGCAAAATCAAGCTGTTTTCTTTTGAAAGTTC |
| | | AAACTCATCAAGATTATGCTGCTCACTCTTATCATTCTGT |
| | | TGCCAGTAGTTTCAAAATTTAGTTTTGTTAGTCTCTCAGC |
| | | ACCGCAGCACTGGAGCTGTCCTGAAGGTACTCTCGCAG |
| | | GAAATGGGAATTCTACTTGTGTGGGTCCTGCACCCTTCT |
| | | TAATTTTCTCCCATGGAAATAGTATCTTTAGGATTGACAC |
| | | AGAAGGAACCAATTATGAGCAATTGGTGGTGGATGCTGG |
| | | TGTCTCAGTGATCATGGATTTTCATTATAATGAGAAAAGA |
| | | ATCTATTGGGTGGATTTAGAAAGACAACTTTTGCAAAGAG |
| | | TTTTTCTGAATGGGTCAAGGCAAGAGAGAGTATGTAATAT |
| | | AGAGAAAAATGTTTCTGGAATGGCAATAAATTGGATAAAT |
| | | GAAGAAGTTATTTGGTCAAATCAACAGGAAGGAATCATTA |
| | | CAGTAACAGATATGAAAGGAAATAATTCCCACATTCTTTT |
| | | AAGTGCTTTAAAATATCCTGCAAATGTAGCAGTTGATCCA |
| | | GTAGAAAGGTTTATATTTTGGTCTTCAGAGGTGGCTGGA |
| | | AGCCTTTATAGAGCAGATCTCGATGGTGTGGGAGTGAAG |
| | | GCTCTGTTGGAGACATCAGAGAAAATAACAGCTGTGTCA |
| | | TTGGATGTGCTTGATAAGCGGCTGTTTTGGATTCAGTAC |
| | | AACAGAGAAGGAAGCAATTCTCTTATTTGCTCCTGTGATT |
| | | ATGATGGAGGTTCTGTCCACATTAGTAAACATCCAACAC |
| | | AGCATAATTTGTTTGCAATGTCCCTTTTTGGTGACCGTAT |
| | | CTTCTATTCAACATGGAAAATGAAGACAATTTGGATAGCC |
| | | AACAAACACACTGGAAAGGACATGGTTAGAATTAACCTC |
| | | CATTCATCATTTGTACCACTTGGTGAACTGAAAGTAGTGC |
| | | ATCCACTTGCACAACCCAAGGCAGAAGATGACACTTGGG |
| | | AGCCTGAGCAGAAACTTTGCAAATTGAGGAAAGGAAACT |
| | | GCAGCAGCACTGTGTGTGGGCAAGACCTCCAGTCACAC |
| | | TTGTGCATGTGTGCAGAGGGATACGCCCTAAGTCGAGA |
| | | CCGGAAGTACTGTGAAGATGTTAATGAATGTGCTTTTTG |
| | | GAATCATGGCTGTACTCTTGGGTGTAAAAACACCCCTGG |
| | | ATCCTATTACTGCACGTGCCCTGTAGGATTTGTTCTGC |
| | | TTCCTGATGGGAAACGATGTCATCAACTTGTTTCCTGTCC |
| | | ACGCAATGTGTCTGAATGCAGCCATGACTGTGTTCTGAC |
| | | ATCAGAAGGTCCCTTATGTTTCTGTCCTGAAGGCTCAGT |
| | | GCTTGAGAGAGATGGGAAAACATGTAGCGGTTGTTCCTC |
| | | ACCCGATAATGGTGGATGTAGCCAGCTCTGCGTTCCTCT |
| | | TAGCCCAGTATCCTGGGAATGTGATTGCTTTCCTGGGTA |
| | | TGACCTACAACTGGATGAAAAAAGCTGTGCAGCTTCAGG |
| | | ACCACAACCATTTTTGCTGTTTGCCAATTCTCAAGATATT |
| | | CGACACATGCATTTTGATGGAACAGACTATGGAACTCTG |
| | | CTCAGCCAGCAGATGGGAATGGTTTATGCCCTAGATCAT |
| | | GACCCTGTGGAAAATAAGATATACTTTGCCCATACAGCC |
| | | CTGAAGTGGATAGAGAGAGCTAATATGGATGGTTCCCAG |
| | | CGAGAAAGGCTTATTGAGGAAGGAGTAGATGTGCCAGA |
| | | AGGTCTTGCTGTGGACTGGATTGGCCGTAGATTCTATTG |
| | | GACAGACAGAGGGAAATCTCTGATTGGAAGGAGTGATTT |
| | | AAATGGGAAACGTTCCAAAATAATCACTAAGGAGAACAT |
| | | CTCTCAACCACGAGGAATTGCTGTTCATCCAATGGCCAA |
| | | GAGATTATTCTGGACTGATACAGGGATTAATCCACGAATT |
| | | GAAAGTTCTTCCCTCCAAGGCCTTGGCCGTCTGGTTATA |
| | | GCCAGCTCTGATCTAATCTGGCCCAGTGGAATAACGA |
| | | TTGACTTCTTAACTGACAAGTTGTACTGGTGCGATGCCA |
| | | AGCAGTCTGTGATTGAAATGGCCAATCTGGATGGTTCAA |
| | | AACGCCGAAGACTTACCCAGAATGATGTAGGTCACCCAT |
| | | TTGCTGTAGCAGTGTTTGAGGATTATGTGTGGTTCTCAG |
| | | ATTGGGCTATGCCATCAGTAATAAGAGTAAACAAGAGGA |
| | | CTGGCAAAGATAGAGTACGTCTCCAAGGCAGCATGCTGA |
| | | AGCCCTCATCACTGGTTGTGGTTCATCCATTGGCAAAAC |
| | | CAGGAGCAGATCCCTGCTTATATCAAAACGGAGGCTGTG |
| | | AACATATTTGCAAAAAGAGGCTTGGAACTGCTTGGTGTT |
| | | CGTGTCGTGAAGGTTTTATGAAAGCCTCAGATGGGAAAA |
| | | CGTGTCTGGCTCTGGATGGTCATCAGCTGTTGGCAGGT |
| | | GGTGAAGTTGATCTAAAGAACCAAGTAACACCATTGGAC |
| | | ATCTTGTCCAAGACTAGAGTGTCAGAAGATAACATTACA |
| | | GAATCTCAACACATGCTAGTGGCTGAAATCATGGTGTCA |
| | | GATCAAGATGACTGTGCTCCTGTGGGATGCAGCATGTAT |
| | | GCTCGGTGTATTTCAGAGGGAGAGGATGCCACATGTCA |
| | | GTGTTTGAAAGGATTTGCTGGGGATGGAAAACTATGTT |
| | | CTGATATAGATGAATGTGAGATGGGTGTCCCAGTGTGCC |
| | | CCCCTGCCTCCTCCAAGTGCATCAACACCGAAGGTGGTT |
| | | ATGTCTGCCGGTGCTCAGAAGGCTACCAAGGAGATGGG |
| | | ATTCACTGTCTTGATATTGATGAGTGCCAACTGGGGGTG |
| | | CACAGCTGTGGAGAGAATGCCAGCTGCACAAATACAGA |
| | | GGGAGGCTATACCTGCATGTGTGCTGGACGCCTGTCTG |
| | | AACCAGGACTGATTTGCCCTGACTCTACTCCACCCCCTC |
| | | ACCTCAGGGAAGATGACCACCACTATTCCGTAAGAAATA |
| | | GTGACTCTGAATGTCCCCTGTCCCACGATGGGTACTGCC |
| | | TCCATGATGGTGTGTGCATGTATATTGAAGCATTGGACA |
| | | AGTATGCATGCAACTGTGTTGTTGGCTACATCGGGGAGC |
| | | GATGTCAGTACCGAGACCTGAAGTGGTGGGAACTGCGC |
| | | CACGCTGGCCACGGGCAGCAGCAGAAGGTCATCGTGGT |
| | | GGCTGTCTGCGTGGTGGTGCTTGTCATGCTGCTCCTCC |
| | | TGAGCCTGTGGGGGGCCCACTACTACAGGACTCAGAAG |
| | | CTGCTATCGAAAAACCCAAAGAATCCTTATGAGGAGTCG |
| | | AGCAGAGATGTGAGGAGTCGCAGGCCTGCTGACACTGA |
| | | GGATGGGATGTCCTCTTGCCCTCAACCTTGGTTTGTGGT |
| | | TATAAAAGAACACCAAGACCTCAAGAATGGGGGTCAACC |
| | | AGTGGCTGGTGAGGATGGCCAGGCAGCAGATGGGTCAA |
| | | TGCAACCAACTTCATGGAGGCAGGAGCCCCAGTTATGTG |
| | | GAATGGGCACAGAGCAAGGCTGCTGGATTCCAGTATCC |
| | | AGTGATAAGGGCTCCTGTCCCCAGGTAATGGAGCGAAG |
| | | CTTTCATATGCCCTCCTATGGGACACAGACCCTTGAAGG |
| | | GGGTGTCGAGAAGCCCCATTCTCTCCTATCAGCTAACCC |
| | | ATTATGGCAACAAAGGGCCCTGGACCCACCACACCAAAT |
| | | GGAGCTGACTCAGTGAAAACTGGAATTAAAAGGAAAGTC |
| | | AAGAAGAATGAACTATG |
| SEQ ID | Linker sequence | TSGSGSGSGSGSGSGGT |
| NO: 8 | | |

The section headings used herein are for organizational purposes only and are not to be construed as limiting the subject matter described.

Disclosed herein is a bacteriophage engineered to present an epidermal growth factor receptor (EGFR)-binding moiety on the bacteriophage cell surface.

Also disclosed is a bacteriophage for infiltrating a tumour microenvironment of an epidermal growth factor receptor (EGFR)-positive tumour. In an embodiment, the bacteriophage comprises a polypeptide comprising a targeting moiety for binding the extracellular domain of a target molecule expressed by at least one target cell. In an embodiment, the targeting moiety comprises an EGFR-binding moiety and is present on the surface of the bacteriophage in an amount sufficient for targeting the bacteriophage to the at least one target cell. The at least one target cell is an EGFR-positive tumour cell, the target molecule is EGFR and the target cell is an EGFR-positive tumour cell.

The EGFR-binding moiety is for, or is capable of, binding the extracellular domain of an EGFR. In other words, the EGFR-binding moiety is an EGFR extracellular domain-binding moiety. “An EGFR-binding moiety” is meant to include a plurality of EGFR-binding moieties. The bacteriophage may be a tumour-infiltrating bacteriophage and when the EGFR-binding moiety is presented on its surface, the bacteriophage may be a targeted tumour infiltrating bacteriophage for targeting and/or infiltrating an EGFR-positive tumour.

By “present” it is meant that the bacteriophage cell surface comprises the EGFR-binding moiety such that it is capable of binding the extracellular domain of an EGFR. The bacteriophage may be engineered to present the EGFR-binding moiety on the cell surface by techniques known to the person of skill in the art. The EGFR-binding moiety may, for example, be presented using phage display, wherein the EGFR-binding moiety is expressed. In another example, the EGFR-binding moiety may be presented on the cell surface by conjugating the EGFR-binding moiety to a phage capsid protein on the bacteriophage cell surface.

In an embodiment, in addition to being able to bind the extracellular domain of the EGFR, the EGFR-binding moiety comprises one or more of the following functional properties; inhibiting dimerization of an EGFR to an Erb family member; competing with EGF for binding of an EGFR; modulating one or more EGFR downstream signaling pathways; promoting internalization of an EGFR into a cell; or a combination thereof. In an embodiment the EGFR-binding is capable of: inhibiting dimerization of an EGFR to an Erb family member; competing with EGF for binding of an EGFR; modulating one or more EGFR downstream signaling pathways; promoting internalization of an EGFR into a cell; or a combination thereof. By “modulating” it is meant that the EGFR-binding moiety upon binding an EGFR can inhibit or promote EGFR downstream signaling pathways. In an embodiment, the EGFR-binding is capable of: inhibiting dimerization of an EGFR to an Erb family member; competing with EGF for binding of an EGFR; modulating one or more EGFR downstream signaling pathways; and promoting internalization of an EGFR into a cell.

In an embodiment, the bacteriophage is an isolated bacteriophage. In an embodiment the bacteriophage is a lytic bacteriophage. In an embodiment the bacteriophage is prepared using a lytic phage display system. In an embodiment, the bacteriophage is a bacteriophage that is capable of lytic phage display. In an embodiment, the bacteriophage is a member of the Podoviridae, Siphoroviridae, or Myoviridae family. In an embodiment, the bacteriophage is lambda (λ), T4 or T7 bacteriophage. In an embodiment, the bacteriophage is a lambdoid phage. In an embodiment, the lambdoid phage comprises phage 21, 434, 933W, λ, λvir, ϕ27, ϕ80, ϕ82, ϕKO2, ϕE125, D3, Gifsy-1, Gifsy-2, H-19B, HB-4, HK97, HK022, Fels-1, N15, P22, PA-2, PY54, Sf6, ST64T, SfV, or VT2-Sa. In an embodiment, the lambdoid phage is phage λ. In an embodiment, the bacteriophage is λF7.

In an embodiment the bacteriophage is for infiltrating an EGFR-positive tumour in a targeted manner. In an embodiment the EGFR-binding moiety is for targeting the bacteriophage to a tumour expressing EGFR. In an embodiment the

In an embodiment, the epidermal growth factor receptor (EGFR)-binding moiety is selected from the group consisting of an EGFR ligand, a peptide, an aptamer, an anti-EGFR antibody, and a functional variant thereof. In an embodiment, the EGFR-binding moiety is EGF or a functional variant thereof. In an embodiment, the functional variant thereof has one or more of the functional properties of the EGFR-binding moiety. For example, the functional variant the EGFR-binding moiety may inhibit dimerization of an EGFR to an Erb family member; compete with EGF for binding of the EGFR; modulate EGFR downstream signaling pathways; or promote internalization of an EGFR into a cell.

In an embodiment, the EGFR-binding moiety is an EGFR ligand. EGFR ligands bind the extracellular domain of the EGFR and include, for example, epidermal growth factor (EGF), transforming growth factor alpha (TGFalpha), and amphiregulin (AREG). Other EGFR ligands include, for example, heparin-binding EGF (HB-EGF), betacelluin and epiregulin.

In an embodiment, the EGFR-binding moiety is EGF or a functional variant thereof. In a further embodiment, the EGF is human EGF or a functional variant thereof.

In an embodiment the EGF comprises the amino acid sequence as set forth in SEQ ID NO:1 or a variant thereof, wherein the variant thereof comprises an amino acid sequence that is at least 90% identical, such as at least 90%, 95%, 97% 98% or 99% identical to the sequence of SEQ ID NO:1 across the full length thereof.

In an embodiment, the EGFR-binding moiety is fused (for example, conjugated or translationally fused) to a viral capsid protein. In an embodiment, the EGFR-binding moiety is conjugated to a viral capsid protein. In an embodiment, the EGFR-binding moiety is translationally fused to a viral capsid protein. In an embodiment the viral capsid protein is gpD. In an embodiment the EGFR-binding moiety is fused to the C terminus of the capsid protein, for example, gpD. In an embodiment, the EGFR-binding moiety is fused to the N terminus of the capsid protein, for example, gpD. In an embodiment, the capsid protein is capable of high capacity display.

In an embodiment the viral capsid protein is gpD and comprises the amino acid sequence as set forth in SED ID NO:2 or a variant thereof, wherein the variant thereof comprises an amino acid sequence that is at least 90% identical, such as at least 95%, 98% or 99% identical to the sequence of SEQ ID NO:2 across the full length thereof.

In an embodiment a linker is provided between the EGFR-binding moiety and the capsid protein. In certain embodiments, the capsid protein consists of or comprises an amino acid sequence at least about 90%, 95%, 97%, 98%, 99% or 100% identical to the amino acid sequence set forth in SEQ ID NO: 3 or SEQ ID NO:8.

In an embodiment, the bacteriophage comprises a polynucleotide encoding a polypeptide, the polypeptide comprising the EGFR-binding moiety. In an embodiment, the polynucleotide encodes the EGFR-binding moiety. In a further embodiment the polynucleotide encodes a capsid protein and in a further embodiment encodes a linker between the capsid protein and the EGFR-binding moiety.

In an embodiment, the polynucleotide comprises the nucleotide sequence as set forth in SEQ ID NO:4 or a variant thereof, wherein the variant thereof comprises an amino acid sequence that is at least 90% identical, such as at least 95%, 98% or 99% identical to the sequence of SEQ ID NO:4 across the full length thereof.

In an embodiment, the polynucleotide comprises the nucleotide sequence as set forth in SEQ ID NO:5 or a variant thereof wherein the variant thereof comprises an amino acid sequence that is at least 90% identical, such as at least 95%, 98% or 99% identical to the sequence of SEQ ID NO:5 across the full length thereof.

In an embodiment, the polynucleotide comprises the nucleotide sequence as set forth in SEQ ID NO:6 or a variant thereof, wherein the variant thereof comprises an amino acid sequence that is at least 90% identical, such as at least 95%, 98% or 99% identical to the sequence of SEQ ID NO:6 across the full length thereof.

In an embodiment, the polynucleotide comprises the nucleotide sequence as set forth in SEQ ID NO:7 or a variant thereof, wherein the variant thereof comprises an amino acid sequence that is at least 90% identical, such as at least 95%, 98% or 99% identical to the sequence of SEQ ID NO:7 across the full length thereof.

In an embodiment, the bacteriophage comprises a polypeptide encoded by the polynucleotide sequence as set forth in SEQ ID NO:4, SEQ ID NO:5, SEQ ID NO:6 or SEQ ID NO:7.

In an embodiment, the EGFR-binding moiety is present on the bacteriophage cell surface in an amount sufficient for targeting the bacteriophage to an EGFR-positive tumour. In an embodiment, the EGFR-binding moiety is present on the bacteriophage cell surface in an amount sufficient for targeting the bacteriophage to the tumour microenvironment of an epidermal growth factor receptor (EGFR)-positive tumour. In an embodiment, the EGFR-binding moiety is present on the bacteriophage cell surface in an amount sufficient for inhibiting growth of an EGFR-positive tumour.

In an embodiment, the EGFR-binding moiety is present on the bacteriophage cell surface in an amount sufficient to bind target EGFR on at least two separate EGFR-positive tumour cells. In an embodiment the EGFR-binding moiety is present on the bacteriophage cell surface in an amount sufficient to entrap the bacteriophage between at least two tumour cells. In an embodiment the EGFR-binding moiety is present on the bacteriophage cell surface in an amount sufficient to bind target EGFR on more than two target cells.

In an embodiment the bacteriophage comprises a high surface decoration of the EGFR-binding moiety.

In an embodiment, a high surface decoration of the EGFR-binding moiety is an amount of surface EGFR-binding moiety that is increased by 40% or more than the amount of surface decoration of an EGFR-binding moiety expressed on the surface of a λF7 bacteriophage generated by phage display in an amber suppression strain SupF E. coli host and culturing the SupF E. coli host cells at 37 degrees. In an embodiment, a high surface decoration is an amount of surface EGFR-binding moiety that is equivalent to or greater than the amount of surface decoration of an EGFR-binding moiety expressed on the surface of a λF7 bacteriophage generated by phage display in an amber suppression strain SupD E. coli host and culturing the SupD E. coli host cells at 37 degrees.

In an embodiment, the bacteriophage is a recombinant bacteriophage. In an embodiment the bacteriophage is a mutant phage that includes a nonsense mutation which prevents expression of the selected endogenous capsid protein as a functional protein. Expression of functional capsid protein is prevented by inclusion of a nonsense mutation within the capsid-encoding region in the phage genome. Nonsense mutations include, for example, amber mutations, ochre mutations and opal mutations. Phage mutants include, for example, amber mutants which possess a premature UAG or TAG codon in the RNA or DNA phage genome, respectively, within the capsid-encoding region; ochre mutants which include a premature UAA or TAA codon in the RNA or DNA phage genome, respectively, within the capsid-encoding region; and opal mutants which include a premature UGA or TGA codon in the RNA or DNA phage genome, respectively, within the capsid-encoding region. Examples of suitable host cells include bacterial cells, for example, bacterial cells such as E. coli cells, Enterobacter sp., Pseudomonas sp. and Klebsiella sp. The host cell is infected with a phage adapted to express a peptide of interest fused to a functional phage capsid protein. The phage may be modified to incorporate a nucleotide encoding an EGFR-binding moiety-capsid protein fused product within its genome, or may incorporate an expression vector prepared to encode the EGFR-binding moiety fused product. In a lytic phage display system the host cell is lysed and surface decorated bacteriophages are released from the cell. Well-established recombinant technology is used to prepare a phage to express a fused product.

In an embodiment a plurality of the bacteriophage disclosed herein together may be formulated with a pharmaceutically acceptable excipient, diluent, or carrier to provide a pharmaceutical composition.

In an aspect, there is provided an EGFR-positive tumour-targeting composition comprising a plurality of bacteriophage engineered to present an epidermal growth factor receptor-binding moiety on the bacteriophage cell surface, together with a pharmaceutically acceptable excipient, diluent, or carrier.

In an embodiment the pharmaceutical composition comprises about 10⁷ to about 10⁹ PFU bacteriophage per ml of total volume of the composition. In an embodiment, the pharmaceutical composition comprises at least 10⁷ PFU of bacteriophage per ml of total volume of the composition. In an embodiment the composition comprises at least 10⁸ PFU of bacteriophage per ml of total volume of the composition. In an embodiment the composition comprises at least 10⁹ PFU of bacteriophage per ml of total volume of the composition. In an embodiment, the composition comprises about 10¹⁰ PFU of bacteriophage per ml of total volume of the composition.

In an embodiment, the pharmaceutical composition comprises bacteriophage in an amount sufficient to reduce a growth of an EGFR-positive tumour. In some embodiments, the bacteriophage are in an amount effective to increase the penetration of the plurality of bacteriophage into the tumour. Alternatively and/or additionally, the bacteriophage are in an amount effective to increase an internalization of the plurality of bacteriophage into tumour cells and/or cells of the tumour microenvironment. Alternatively and/or additionally, the bacteriophage are in an amount sufficient to increase accumulation of the plurality of bacteriophage in the tumour.

Any suitable method or route can be used to administer the bacteriophages or pharmaceutical composition described herein. In an embodiment, the pharmaceutical composition is formulated for intravenous administration, intratumoural administration or rectal administration. In certain embodiments, the bacteriophages or composition described herein are administered intravenously. In certain embodiments, the compositions or bacteriophages described herein are administered intratumourally. In certain embodiments, the compositions or bacteriophages described herein are administered rectally. In any of the above aspects, administration via one route may be combined with one or more other routes of administration. Administration of the bacteriophage via the different routes may be sequential and/or simultaneous.

Disclosed herein is a kit for use in treating an EGFR-positive tumour in a mammalian subject, wherein the kit comprises a bacteriophage disclosed herein or a pharmaceutical composition disclosed herein and instructions for administering the bacteriophage or pharmaceutical composition to a subject.

Disclosed herein is a commercial package for use in treating an EGFR-positive tumour in a mammalian subject, wherein the commercial package comprises a bacteriophage as disclosed herein or a pharmaceutical composition disclosed herein and instructions for administering the bacteriophage or pharmaceutical composition to a subject.

In an embodiment, the bacteriophage or the composition comprising the bacteriophage is for use in inhibiting the growth of an EGFR-positive tumour. In an embodiment, the bacteriophage or the composition comprising the bacteriophage is for use in penetrating a tumour microenvironment of an EGFR-positive tumour. In an embodiment, the bacteriophage is for internalization within a cell of an EGFR-positive tumour. In an embodiment the bacteriophage or composition is for use in targeting and infiltrating a tumour microenvironment of an EGFR-positive tumour. In an embodiment the EGFR-binding moiety is for promoting the internalization of the bacteriophage within a cell of an EGFR-positive tumour or a cell of the tumour microenvironment of an EGFR-positive tumour.

In an embodiment, the bacteriophage or the composition is for use in delivering a biological entity to a tumour microenvironment, a tumour interstitial space and/or a tumour interstitium. In an embodiment, the bacteriophage or the composition is for use in targeting a biological entity to a tumour microenvironment of an EGFR-positive tumour, wherein the biological entity is the bacteriophage. In an embodiment, the bacteriophage is for internalization within a tumour cell of a solid tumour. In an embodiment, the bacteriophage disclosed herein or the pharmaceutical composition disclosed herein is used in a method of treating an EGFR-positive tumour in a subject in need thereof comprising administering to the subject a bacteriophage disclosed herein or a pharmaceutical composition disclosed herein in a dose effective to treat the tumour. The tumour is an EGFR-positive tumour.

Also disclosed is a method of treating a tumour in a subject in need thereof. The method comprises administering to the subject a therapeutically effective amount of a composition comprising a plurality of bacteriophage engineered to present an epidermal growth factor receptor (EGFR)-binding moiety on the bacteriophage cell surface in a dose effective to treat the tumour, wherein the tumour is an EGFR-positive tumour. In some embodiments, treating comprises reducing a growth of the EGFR-positive tumour.

Also disclosed is a method of delivering a biological entity to a tumour microenvironment of an epidermal growth factor receptor (EGFR)-positive tumour in a subject in need thereof, the method comprising: administering to the subject a dose of the bacteriophage as disclosed herein or the pharmaceutical composition as disclosed herein, wherein the bacteriophage is the biological entity.

In an embodiment the tumour is a solid tumour. In an embodiment the tumour has a genetic signature comprising wildtype KRAS and mutated BRAF. In an embodiment, the tumour is a breast, lung, colon, stomach, pancreas, ovary, cervix, brain, head, neck, head and neck, prostate or kidney tumour. In an embodiment, the tumour is a lung tumour and the bacteriophage or composition is for treating non-small cell lung cancer, or lung adenocarcinoma. In an embodiment, the tumour is a colon tumour and the bacteriophage or composition is for treating metastatic colorectal cancer or a colorectal carcinoma. In an embodiment, the tumour is a colorectal carcinoma.

In an embodiment, the disclosed bacteriophage or pharmaceutical composition is for use in the manufacture of a medicament for treating an EGFR-positive tumour in a subject in need thereof. In an embodiment, the tumour is a solid tumour or a spheroid. In an embodiment, the solid tumour is a breast, lung, colon, stomach, pancreas, ovary, cervix, brain, head, neck, skin, prostate or kidney tumour. In an embodiment the composition is for treating colorectal carcinoma, non-small cell lung cancer, lung adenocarcinoma, squamous cell carcinoma or large cell carcinoma. In an embodiment the composition is for treating metastatic colorectal cancer or an colorectal adenocarcinoma. In an embodiment, the tumour is an EGFR-positive, wildtype KRAS and mutated BRAF tumour. In an embodiment, the medicament is for intravenous administration, intratumoural administration or rectal administration to the subject.

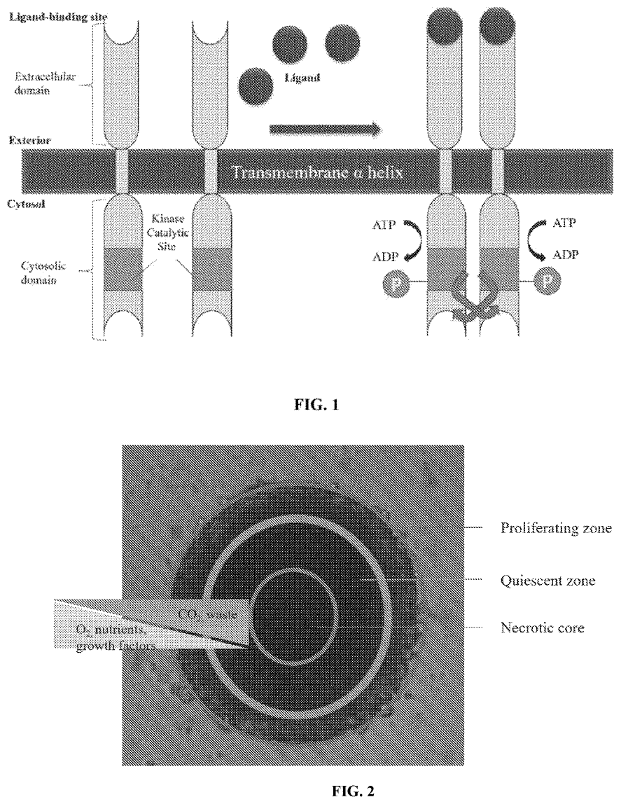

Epidermal growth factor receptor and other ErbB family members: Despite expression in normal cells, the epidermal growth factor receptor (EGFR, also known as ErbB1, HER1) is a transmembrane glycoprotein that has become an attractive target in cancer therapy, as it is often overexpressed in epithelial tumours and contributes to tumour progression. EGFR modulates growth, signaling, differentiation, adhesion and migration of cancer cells. Higher expression and ligand-independent signaling mutations of EGFR have been implicated in many cancer cells, including metastatic colorectal cancer cells, glioblastoma, pancreatic cancer, and breast cancer. ErbB family members consist of not only EGFR, but also ErbB2 (HER2), ErbB3 (HER3) and ErbB4 (HER4), which are all structurally related. Each is composed of an extracellular ligand binding domain, transmembrane domain, tyrosine kinase domain, and a tyrosine-containing C-terminal tail (FIG. 1, showing that The extracellular binding domain of receptor tyrosine kinase binds to ligands, which can cause the receptor to dimerize with another RTK. This activates the kinase activity in the cytosolic domain of the RTK and induces auto-phosphorylation of the adjacent dimerization partner). EGFR and ErbB4 can each be bound by a ligand and autophosphorylated through intracellular tyrosine kinase domains. ErbB2, however, does not bind to ligands directly but is the preferred dimerization partner for EGFR. ErbB3 does not possess intrinsic tyrosine kinase activity but signal transduction can occur through dimerization with ErbB2. There are various ligands that bind to the extracellular ligand-binding domain of EGFR that ultimately contribute to the signaling diversity of EGFR pathways. Examples of EGFR ligands include epidermal growth factor (EGF), transforming growth factor-α (TGF-α), amphiregulin. Neuregulin-1 and neuregulin-2 can bind to ErbB3 and both ErbB3 and ErbB4, respectively.

Upon binding of a ligand, EGFR dimerizes, which is an essential step for the activation of the protein-kinase in the cytosolic domain and the subsequent phosphorylation of the tyrosine residues on the adjacent dimer partner (FIG. 1). The tyrosine phosphorylation of EGFR creates binding sites for Grb2 and Src homology 2 (Shc2), activating downstream signal-transduction events, such as the Ras/MAPK, PLCyl/PKC, PI(3)kinase/Akt, and STAT pathways. This consequently alters cellular physiology and gene expression. Gene expression may also be influenced by ErbB family members that escape the early endosomes and localize in the nucleus, although the mechanism is currently unclear. Activated receptors bound by ligands are most likely internalized via clathrin, whereby the receptors detach from the clathrin-coated pits and are directed to the lysosomes for degradation. Unbound EGFR is recycled more rapidly to the surface. Ligand-bound EGFR may either be degraded in the lysosomes or de-ubiquintinated then recycled, depending on the specific receptor-ligand combination. EGFR heterodimerization with ErbB2 can assist in the evasion of the lysosome; these receptors are recycled to the cell surface instead, increasing EGFR surface density and consequently the downstream signaling events due to prolonged EGFR autophosphorylation. Signal attenuation is initiated by dephosphorylation via phosphatases, such as density-enhanced phosphatase-1 and protein tyrosine phosphatase PTP1B.

EGFR is expressed in many types of cancers, and generally will reflect a more aggressive histological and clinical behavior of the cancer. EGFR is expressed in epithelial tumours such as non small cell lung cancer, colorectal cancer and head and neck tumours. EGFR is expressed in cancers including, for example, lung, ovarian, breast, colon, pancreatic, brain, esophageal, synovial sarcoma, and head and neck cancer. There are many therapies that have been approved for treating EGFR-positive tumours, including, for example, antibody therapies that target the extracellular domain of the EGFR and tyrosine kinase inhibitors that inhibit downstream signaling pathways.

EGFR in cancer therapy: Clinical efficacy often depends on the density of EGFR expressed by cells and the effective blockage. The extracellular domain of EGFR is accessible to monoclonal antibodies; the binding of these antibodies inhibits the intracellular signal transduction pathways that can then respond to angiogenic factors. Mechanisms of action of anti-EGFR therapeutics include physically blocking the ligand from binding (primarily antibodies) or inhibiting the tyrosine kinase enzymatic activity (small molecular weight tyrosine kinase inhibitors), which prevents autophosphorylation and downstream signaling events. Gefitinib (Iressa®) is a clinically-approved EGFR tyrosine kinase inhibitor, that has shown to suppress tumour growth at the end of a 4-week treatment period of 5 mg/dose in GEO cancer xenografts in immunodeficient mice (Ciardiello et al., 2000). Cetuximab (Erbitux®), panitumumab (Vectibix®) and necitumumab (Portrazza®) are examples of chimeric mouse/human monoclonal antibodies that target the extracellular domain of the EGFR and that are approved for treatment of EGFR-positive cancers, including colorectal cancer, squamous cell carcinoma of the head and neck and non-small cell lung cancer. Antibodies directed to the EGFR competitively inhibit the binding of epidermal growth factor (EGF) and other ligands, such as transforming growth factor-alpha, thereby preventing dimerization of the EGFR with EGFR or Erb family member and/or inhibiting downstream signaling pathways controlling cell growth, survival and/or other process related to cancer growth, spread and survival.

Cetuximab has demonstrated the ability to suppress xenograft tumour growth and has been approved for clinical use against colorectal cancer, metastatic non-small cell lung cancer head and neck cancer. Upon binding of cetuximab, EGFR has been shown to dimerize, but is not accompanied by the phosphorylation of downstream proteins, such as Akt and Erk, which are essential in regulation of cell proliferation and survival. Binding of a ligand, such as EGF, will induce a rapid EGF-EGFR complex turnover. The occupation of EGFR will be reduced and consequently, interactions with signaling proteins that perpetuate the active state of the intracellular signaling pathways will also be reduced. The enzymatic tyrosine kinase function can also be inhibited by low molecular weight molecules that compete for the intracellular Mg-ATP binding site to prevent further downstream intracellular signalling. Gefitinib (Iressa®) and Erlotinib (Tarceva®) are examples of EGFR tyrosine kinase inhibitors used as first-line treatments of advanced non-small cell lung cancer, which can achieve prolonged responses to treatment in chemotherapy-naïve patients.

HT29 colon adenocarcinoma and EGFR expression: The colon adenocarcinoma cell line, HT29 expresses relatively high levels of EGFR and possesses wildtype KRAS and mutated BRAF. KRAS serves as an effector molecule for signal transduction from a ligand-bound EGFR to the nucleus for changes in gene expression; mutations in KRAS have displayed persistent downstream signaling without the influence of EGFR and high resistance to EGFR-targeted monoclonal antibody therapy. Clinical trial results have revealed that the addition of cetuximab to first-line chemotherapy is beneficial to patients with the wildtype KRAS gene. BRAF is a downstream effector of RAS in the EGFR pathway, promoting cell proliferation and survival through the constitutive MAPK signaling pathway. The conventional criteria for cetuximab therapy is currently EGFR-positive and KRAS wildtype; colorectal cancer patients with the BRAF mutation may be considered to be a minor ‘non-effective’ group therapy as determined by clinical trials. BRAF or KRAS mutations decrease the effectiveness of EGFR-targeting therapeutics, as the mutations enable constitutive cell proliferation and survival. Therefore, KRAS mutations, and BRAF mutations to a lesser degree, typically serve as markers of resistance to anti-EGFR therapy, as patients have shown to express a lower response rate than those with wildtype tumours. Mouse HT29 cell xenografts displayed high sensitivity to cetuximab and mitogen-activated protein kinase (MEK) inhibitor, selumetinib (AZD2644). By inhibiting MEK, selumetinib further inhibited extracellular signal-related kinase (ERK) phosphorylation and tumour cell proliferation. The combination of cetuximab reactivated MAPK signaling, resulting in the significant inhibition of HT29 cell xenograft growth.

The inventors found that EGFR signal transduction is altered by cetuximab in HT29 cells. The level of phosphorylation by growth factors, TGF-α, IGF and EGF, decreased in the presence of cetuximab in HT29 cells and partially inhibited the MAPK pathway (Matsuo et al., 2011). Others showed by scanning electron microscopy, significant microvilli and filopodia reduction on HT29 cells in response to cetuximab. These cells lost boundary contacts with other cells as a result, which are indicative of differentiation toward apoptosis. Downregulation in MAPK signaling, apoptosis and phosphatidylinositol signaling system of HT29 cells is also reported in the same study.

3D Cultures: Clinically, targeting tumour cells without affecting normal cells is a major goal in cancer therapy as this would reduce side effects for patients. Low-weight molecules with passive and/or active tumour targeting have been investigated for potential clinical applications. In vitro cell cultures provide a highly controlled environment; 3D tissue models, such as cells grown on structured scaffolds, multicellular layers and multicellular tumour spheroids, have been particularly useful in studying drug and nanoparticle penetration in vitro as they provide better representation of the complexity and state of cancer cells in their native environment. While conventional monolayer cultures may be a convenient and an effective way for predicting molecular targets and pathways, they are unable to completely recapitulate the complexity and heterogeneity of tumours. Previous studies have shown that while certain apoptosis-inducing drugs are effective against monolayer cell cultures, they failed to achieve the same level of cytotoxicity in 3D cultures, which may be analogous to the chemoresistance often observed in vivo.

Multicellular spheroids: Multicellular tumour spheroids (MCTS) serve to bridge the gap between monolayer cultures and animal models by effectively mimicking the pathophysiological environment and morphology of in vivo solid tumours and demonstrating similar responses to therapy. They are preclinical models that re-establish the complex network of cells, ECM, gene expression, cell signals, gradients of pH, oxygen, metabolism and proliferation found in solid tumours. The cells in MCTS are concentrically-arranged whereby the outer region is comprised of proliferating cells that are abundantly exposed to nutrients and oxygen (FIG. 2). These cells surround quiescent cells, closer to what is a necrotic core if the MCTS beyond a critical size of 500 μm in diameter. These characteristics of spheroids ultimately influence protein expression, binding and penetration of drugs through the MCTS. Some molecular targets also become upregulated, which have previously been exploited for selective targeting.

The inventors found that anticancer drugs, melphalan, irinotecan, oxaliplatin and fluorouracil (5-FU), are highly active in HCT116 monolayer cultures, but 6-day-old spheroids are almost completely resistant. These effects are suggested to have been caused by the suppression of genes implicated in DNA replication and cell cycle associated with the 3D culture.

A population of cells in spheroid culture will display stronger resistance to drugs even in the absence of intervening physical barriers (e.g. ECM components), as some cells will be required to be in proliferative state for the drug to induce toxicity. For example, one group treated A549 spheroids with paclitaxel, a microtubule-targeting drug that disrupts cell adhesion, to increase the interstitial spaces between cells to achieve equal access of cells by a chemotherapeutic drug. This is confirmed by observing the effective penetration of 10,000 Da dextrans through spheroids. Despite the increased accessibility of cells for the chemotherapeutic drug pemetrexed, a population of cells remained unperturbed and viable. 3D cultures reproduce the metabolic gradients and altered cell-cell contacts that are more representative of in vivo solid tumours; therefore, MCTS are considered to be more reliable in predicting a drug's therapeutic potential than conventional monolayer cultures in preclinical studies.

Spheroids can be monitored for their change in size and morphology in response to a drug by standard phase-contrast microscopy, while the measurement of drug penetration can be accomplished by confocal microscopy. Typically, spheroids with diameters of up to 300 μm are employed in drug testing, which exhibit well-developed cell-cell, cell-matrix interactions and reproducible results in drug screening. Although the use of larger spheroids is tempting to model the behaviour and characteristics of solid tumours and their response to treatments, it is difficult to achieve reproducible results. The variability of parameters, such as volume, shape and sphericity caused spheroids to display different responses to treatments—all of which can be amplified by larger spheroid size.

In vitro modeling of nanoparticle penetration into spheroids is also affected by external parameters such as temperature and cell type. The penetration efficiency is reduced at lower temperatures as modeled by the equation for Brownian motion. At higher temperatures, the viscosity of the liquid medium decreases, resulting in faster diffusion of nanoparticles. Cell type will affect in variations in packing density and intercellular channel sizes, influencing the overall kinetics of the particle. Certain cells form larger interstitial spaces enabling nanoparticle movement, which does not necessarily reflect the penetrability of the nanoparticle itself. Stromal spheroids will appropriately model the structural barrier that restricts particle movement in vivo. Mikhail et al. reported that despite the treatment of HT29 and HeLa spheroids with the same concentration of docetaxel, different responses are observed; HT29 spheroids consist of more non-proliferating cells with higher packing density than HeLa spheroids. Therefore, it is crucial to screen therapeutic molecules in spheroids generated from various cell types to appropriately assess their kinetic properties.

Bacteriophages: Bacteriophages (phages) are viruses that exclusively infect bacteria, and like plant and animal viruses, phages are unable to propagate in the absence of a host. The bacterial host provides machinery and organic material for phage biosynthesis and packaging. Phages are the most abundant biological entities on earth—an estimate of 1031 particles exist as free virions or within their bacterial hosts. All phages undergo the general process of adsorption to their bacterial host(s), DNA injection, replication, virion production, followed by release.

The human body is extensively colonized by phages, and the ubiquitous nature of phages leads to constant exposure to a diverse spectrum of phages from birth. Phages colonize the skin, lungs, oral cavity, gut, and urinary tract, contributing to an extensive genetic diversity to humans, and ultimately playing an essential role in maintaining our health. The gut microbiota consists mainly of phages, with an estimate of 10⁹ virus-like particles per gram of stool. In healthy humans, this includes double-stranded DNA viruses of the Podoviridae, Siphoviridae and Myoviridae family.

The diversity of phages contributes significantly to the gut microbiota structure and function. Phages may serve as reservoirs of genetic diversity by serving as vehicles for horizontal transfer of virulence and antibiotic resistance for their bacterial host. Additionally, these entities may play a role in the defense of the mucosal barrier against bacteria. The Ig-like domains exposed on the surface of phages have been shown to interact with the mucin glycoproteins to decrease bacterial colonization of the mucosa, thereby reducing bacterial translocation and the potential for bacteremia.

Phages have received enormous amount of attention in the last decade for their potential applications. This includes treatment of bacterial infections as well as its potential for genetic therapy. Phages have no natural tropism for mammalian cells as they would for bacterial hosts and have resided in our bodies throughout hominid evolution in a tripartite relationship with their bacterial and human hosts. For decades, phages have extensively been used in Eastern Europe for treatment of bacterial infections, but have only recently garnered interest for their influence of cancer processes. Phages have demonstrated intrinsic antitumour activities such as slowing the tumour growth. The most widely known example of this is perhaps the attachment of T4 phages to B16 melanoma cells mediated by the Lys-Gly-Asp (KGD) motifs present on the phage capsid with the integrin beta1 and beta3 integrin receptors on target cancer cells. This resulted in decreased tumour metastasis. Administered phages have also shown remarkable penetrative capabilities through various cell types, passing through relatively impermeable physiological barriers in our bodies. This includes the blood-brain-barrier, which often restricts the movement of molecules that are less than 500 Da, lipophilic, and share a structural homology to compounds that enter the brain through active transport. Intraperitoneal and intranasal application of M13 phages in mice have resulted in their recovery in the brain as early as 1 h following administration. Phages can also cross the gut mucosal barrier as orally administered phages have been recovered in the blood of. Administered phages can bypass the gut flora, adhere to the glycoproteins in the mucosa and traverse the epithelium transcellularly or via M cells. Recently, the mechanism for transcytosis across epithelial cells have been described by Nguyen et al. Transcytosis is observed for 0.1% of applied phages in the apical-to-basal direction. Phages applied topically to clear bacterial infections have shown to penetrate through the multiple layers of the skin, from the epidermis to the adipose tissues. Soothill et al. excised and replaced skin grafts extending down to the subcutaneous fat, but the topical application of phages prevented the graft destruction by Pseudomonas aeruginosa, demonstrating the ability of phages to permeate the skin tissue. This unique ability of phages to pass through minute intercellular spaces may lead to effective extravasation, infiltration and accumulation in the tumour microenvironment, possibly representing a huge potential for a drug delivery platform for phages in cancer therapy.

Phages and cancer: Anticancer properties of T4 phages have been reported, demonstrated by the inhibition of lung metastasis of B16 melanoma cells by the binding of T4 and its sub-strain HAP1, mediated through the KGD motif on the gp24 phage capsid protein and to the cell surface β3 integrins. Certain animal viruses, such as adenovirus, HIV, HPEV1 virus and hantavirus, use integrins for cell entry, which are present on platelets, monocytes and some neoplastic cells. The blockade of integrins downregulates its functions, which has been postulated to be the cause of anti-metastatic activity imparted by these phages. Phages in the context of cancer immunotherapy has also been of interest, as the phage-induced tumour regression in mice. Administration of tumour-specific phages secreted proinflammatory cytokines IL-12 and interferon y, resulting in the migration of mouse neutrophils to the site of tumour and potentiating tumour destruction. Arab et al. also studied the immunogenicity of λ phage particles displaying E75 peptide derived from HER2/neu in a TUBO cell xenograft model of BALB/c mice. This resulted in the induction of CD8+T cells for mice that are vaccinated with the phages with significantly reduced the size of the tumour.

Phage internalization and cellular trafficking in eukaryotic cells: Despite the ubiquity of phages in the environment, current knowledge of phage interactions with mammalian cells is very limited. There is evidence that phages can bind to the surface and be internalized by eukaryotic cells, but the penetration of phage DNA into the nucleus, replication and release of progeny particles have not been directly observed. Cells infected with phages harboured about 0.2% of cellular RNA that is k-specific (lambdoid phage-encoded (β-galactosidase). Since then, targeted phage transduction in mammalian cells have been extensively studied by measuring the expression of GFP, neomycin phosphotransferase or β-galactosidase. Phage display can be used to fuse targeting ligands on the phage surface; this combined with a mammalian expression cassette can confer mammalian tropism for the phage. The interactions between targeting ligands and their cognate receptors increase the likelihood of phage-internalization in a concentration-dependent manner, and subsequently their transfection efficiency M13 filamentous phages displaying EGF peptides can transduce COS-1 cells by delivering a GFP gene. Transduction efficiency is shown to be greater by 100-fold than by control phages without any display. Transduction is observed with concentrations as low as 250 phage particles/cell and increased in a dose-dependent manner. This ligand density-dependent increase in transduction efficiency has also been shown with the display of fibroblast growth factor (FGF2) and scFv antibody fragment, targeting the fibroblast growth factor receptor and ErbB2 receptor, respectively. The display of RGD peptides by filamentous phages has also mediated cell internalization via multivalent binding to integrin receptors in vitro. Phages are internalized in less than 15 min, in a process resembling phagocytosis more than receptor-mediated endocytosis. Packaging into clathrin-coated vesicles require particles to be less than 100-150 nm in diameter, but filamentous phages are often between 800-2000 nm in length.

Phage internalization is not contingent on phage surface modifications to display cell-targeting ligands. The ability of E. coli phage PK1A2 to bind specifically to polysialic acid receptors on eukaryotic neuroblastoma cells in vitro, without the mediation of phage surface modifications. Furthermore, there is strong evidence that this induced internalization of the phage-polysialic acid complex; fluorescent phages appeared intracellularly as early as 30 min after incubation and increased up to 24 h. Polysialic acid bears structural similarity to a bacterial receptor that mediates phage entry. It has been postulated by the authors that there are other undiscovered surface epitopes on eukaryotic cells that can be bound specifically by phages, due to an abundance of structurally-similar carbohydrates shared between pathogenic bacteria and host eukaryotic cells. Following cellular uptake, phages are likely directed to the endolysosomal pathway, losing infectivity in the lysosomes.

Phage display: Although wildtype T4 phages without any surface modifications have demonstrated antitumour activities, phages can be engineered to enhance their application and efficacy profile. Tropism for specific cells can be conferred by fusing cell-targeting ligands to surface proteins. This is known as phage display technology, whereby a peptide or protein coding gene is translationally fused to the capsid gene of the phage. As a result, the phage product will carry the gene for the protein/peptide of interest and will be capable of replication within the construct. This approach has been extremely powerful in isolating ligands for drug discovery, identifying protein-protein interactions and antibody fragments, epitope mapping, as well as phage engineering for specific targeting of tissues and cells. Peptides with desired binding profiles can be selected for in a process called “biopanning,” which has further progressed to live animal ‘in vivo panning’. This approach has since been extensively investigated in cancer therapy; tumour-specific ligands have been screened and isolated by their affinity and specificity to neoplastic cells and vascular endothelium. While monoclonal antibodies can also achieve high affinity and specificity, their large size is a limiting factor for effective penetration into the tumour tissue.

Much success has been achieved by utilizing isolated peptides from screening phage display libraries for delivery of nanoparticles to tumours. Several peptides have been identified to bind to HER2, EGFR, IL-6 receptor. Furthermore, peptides isolated from phage-display peptide libraries have progressed to phase III clinical trials, such as Amgen's AMG-386, an anti-angiogenic therapeutic for treatment of various cancers, and Romiplostim (AMG 531), drug approved by the FDA for the treatment of thrombocytopenia.

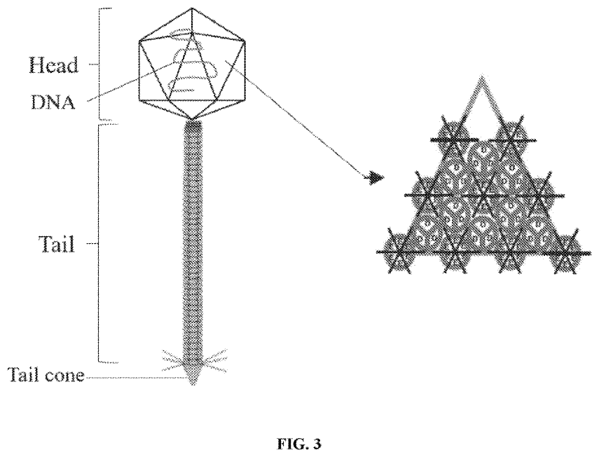

Tunable λ. phage display: Bacteriophage λ, is a temperate phage in the family of Siphoviridae viruses, with icosahedral capsid (−50 nm in diameter), long fibrous tail (150 nm in length) and double-stranded genome (−48 kb). The capsid consists of two major coat proteins, gpE and gpD, which are responsible for the structure of the prohead and stability during genome packaging, respectively (FIG. 3, showing that the capsid head is composed of gpD and gpE proteins incorporated into trimers and hexamers, respectively. λ. icosahedral head comprises a total of 20 faces and 405-420 of each gpD and gpE proteins). gpD consists of 10⁹ amino acids and cuts the concatemers at the cohesive end site (cos) during packaging of the DNA into the phage prohead while simultaneously stabilizing it. gpD-deficient mutants result in 82% of the DNA content of wildtype phage. Phage λ is able to carry large DNA fragments, has high infectivity and offers several advantages over filamentous phages. It is similar in size and shape to mammalian viruses, has double-stranded DNA genome, and despite its small size, the mature λ-head can package large amounts of DNA fragments of up to 50 kb. It can also accommodate larger exogenous proteins with a high copy number, conferring high avidity for a target receptor. Phage λ can be employed for phage display via both gpD, and the tail protein, gpV. There are 415 copies of gpE and 405-420 copies of gpD. Lytic phages, including phage λ, possess a more efficient display system compared to filamentous phage, with greater gpD fusions of the same polypeptide by 10⁴-fold. Examples of lytic phage display systems can be found at least in Nicastro et al., 2013 and US 2015/0031583.

Immune response to phage therapy: Immune response to phage administration can be influenced by: i) the size and number of surface epitopes; ii) route of administration; iii) dose and frequency of phage administration and iv) patient history of exposure. Antibodies against naturally-occurring phages, such as T4, are detected in the sera of humans. In one study, 50 healthy humans are tested for phage antibodies, of which 81% are found to possess IgG antibodies against the T4 capsid proteins—gp23, gp24 and Soc. This is most likely a result of natural contact with phages in the environment.

Oral phage administration has typically shown weak immune responses in humans, with only marginal changes in serum anti-phage antibodies. Intravenous injection of phages, however, result in the rapid phage clearance from the reticuloendothelial system (RES). The RES is comprised of phagocytic cells that reside in the lymph nodes and the spleen, including Kupffer cells (specialized mononuclear phagocytes) of the liver. These cells remove foreign phage particles, posing a significant challenge to the maintenance of applied phage titers in vivo. As a result, significant titers of phages have been recovered from the spleen in mice, regardless of the route of administration. The half-life of phages in circulation often depend on phage surface protein sequences; Merril et al. showed the ability to select for mutant a, phages that are able to persist in circulation for several hours by effectively evading Kupffer cells in mice.

There have been reports of both pro- and anti-inflammatory cytokine release in response to phages. The effects of phages on the tumour necrosis factor-α (TNF-α) and interleukin 6 (IL-6) levels in the sera of 51 patients with suppurative infections has been studied. Long-term treatment with phages led to elevated levels of these cytokines; however, like many other similar studies testing cytokine levels in response to phage therapy, it is unclear whether the short-term normalization or reduction in the proinflammatory cytokine levels are influenced by the reduced burden of pathogenic bacteria in the body. In addition, changes of 12 proinflammatory cytokines including ILL IL2, IL10, IL17, IFN-y and TNF-α in response to orally fed T7 phage in mice has been monitored and found that only IL17A is slightly elevated (less than 10%) compared to the control group that is fed SM buffer. Histopathological analysis of the same study showed no noticeable differences in the tissue samples of the gastrointestinal tract. Anti-inflammatory action of phages has also been reported; a study used highly purified Staphylococcus aureus and Pseudomonas aeruginosa phages to stimulate peripheral blood mononuclear cells (PBMCs) derived from healthy human donors. The genes for IL1RN (an anti-inflammatory IL1 receptor antagonist) and SOCS3 (suppressor of cytokine signaling 3) are upregulated, while proinflammatory cytokines such as ILIA and IL1B, are also elevated. Therefore, phages appear to activate several immunological pathways following administration.

Phage penetration of solid tumours: The ability of phages to penetrate through the various regions of solid tumours may depend on several factors including the size and morphology of the phage, surface charge of the phage, interactions with the ECM, and the potential binding and internalization to the eukaryotic cells. Diffusing particles often interact with molecules during their transit through intercellular spaces, although outcomes will vary significantly depending on whether the interaction is specific or non-specific. Specific interactions, such as those of monoclonal antibodies and tumour antigens, can impair tumour penetration by binding to external cells, resulting in poor biodistribution in the solid tumour in what is known as the “binding site barrier”. This effect is often dependent on the binding affinity and antigen density, and is often the cause of poor nanoparticle diffusion and unintended internalization by stroma cells near the blood vessels.

Recently, E. coli phage lysate enriched from sewage has been reported to bind to three major macromolecules of the ECM—gelatin (denatured collagen), fibronectin, and heparin by column-based assays. ECM biochemically impedes vector access to tumour cells. The same phage lysate is used to test their ability to protect HT29 cells against infection by pathogenic host bacteria. Phage adherence to HT29 cells is evidenced, as phages are able to reduce the bacterial load by 48%, even after washing of the cells to discard unbound phages. These phages did not show signs of cytotoxic effects on HT29 cells by the Resazurin assay.

Adeno-associated virus/phage (AAVP) is a hybrid phage vector with a mammalian transgene cassette harbouring inverted terminal repeats from adeno-associated virus 2 as well as a peptide display vector from fd phage genome. The AAVP displays iRGD that enables homing and entry to av integrin receptors and has been used widely for gene delivery into cancer cells (Hajitou et al., 2007; Przystal et al., 2013; Smith et al., 2017). Despite the narrow shape (6.5 nm×1400 nm) of AAVP, diffusion through the ECM-gel matrix is still very poor. Infiltration, internalization and gene expression are significantly improved after the depletion of ECM constituents with collagenase and hyaluronidase. Bacteriophage penetration through a MCTS has not previously been clearly characterized but remains to be a parameter that needs to be clearly defined in the development of a drug delivery platform for phages.

A prominent problem with cancer gene therapy is the incomplete eradication of tumours, which frequently results in the recurrence of cancer. Bacteriophages have naturally demonstrated the ability to bind to the surface of mammalian cells as well as the capacity to penetrate different cell type layers and various physiological barriers in the human body. Phages may be able to overcome physical barriers commonly presented in solid tumour drug delivery, including high packing density of constituent cells and the high interstitial fluid pressure. Therefore, the inventors sought to understand whether unmodified bacteriophage λ, can passively target and diffuse within multicellular spheroids generated from NIH3T3 mouse fibroblasts. Fibroblasts are stromal cells that secrete connective tissue components, hindering drug penetration into solid tumours.

Bacteriophages (phages), which are bacterial viruses, have a strong potential as a drug delivery vehicle and offer new avenues in anticancer therapy. Tumor-homing peptides and ligands of overexpressed receptors on tumor cell surfaces can be fused to phage capsid proteins for targeting. Phages are unlikely to undergo alterations in their tropism, which is a major concern with the exploitation of oncolytic viruses in anticancer therapy. Phages have demonstrated the ability to bypass the epithelium and endothelium and can accumulate in various mammalian tissues and organs following their administration. Additionally, the production of phages is relatively low, and cost of phage purification appears to be declining with the advancements in technology. Although transfection with phages for anticancer therapy has been explored, the interaction of phages with neoplastic cells in 3-dimensional cell culture and their targeted infiltration and internalization within the complex microenvironment has not yet been described to date. Particularly, the inventors have shown that three dimensional cultures replicate the pathophysiological environment and morphology of in vivo solid tumours and demonstrate similar responses to therapy.

Materials and Methods

Buffers and Solutions

Ampicillin Stock (Sigma Aldrich, Oakville, Canada): This is used as a selective marker for the pPL451 plasmids in the E. coli hosts and is added to broth at 100 m/m1 from a stock prepared at a concentration of 50 mg/ml into filter-sterile water. This stock powder is stored at −20° C.

Luria Bertani Broth: LB broth is a nutritionally-rich medium used for the rapid propagation of bacteria in culture. It consists of 10 g tryptone, 5 g yeast abstract and 5 g NaC1 dissolved in 1 L MilliQ H₂O, sterilized (autoclave) and stored at room temperature.

LB Agar: LB agar is used as a solid support for the growth of bacterial cultures. It consists of 10 g tryptone, 5 g yeast abstract, 5 g NaC1 and 13 g Grade A Bacto™ agar (Difco Laboratories) dissolved in 1 L MilliQ H2O. It is sterilized, poured onto sterile plates and stored at 4° C.

LB Top Agar: LB top agar is used as an overlay on top of regular nutrient agar plates. The semi-solid consistency permits progeny phages to diffuse through the media to infect the evenly-dispersed, “lawn” of bacterial culture. It consists of 10 g tryptone, 5 g yeast abstract, 5 g NaC1 and 7 g Grade A Bacto™ agar, dissolved in 1 L MilliQ H2O, sterilized and stored in 52° C. water bath to maintain the molten state.

PEG (Polyethylene Glycol): PEG is used to precipitate phage particles and purification. It is prepared by dissolving 0.2 M PEG 8000, 0.15 M NaC1 in 1 L MilliQ H₂O (pH 7.8), filter-sterilized and stored at room temperature.

TN Buffer: Phages are stored in TN buffer at 4° C. It is prepared by dissolving 0.1 M NaC1, 0.01 M Tris-HC1 (pH 7.8) and autoclaving afterwards. This is stored at room temperature.

TN/EDTA Buffer: This buffer is used to test for phage sensitivity. 0.1 M NaC1, 0.01 M Tris-HC1, 0.1 M EDTA is dissolved in 1 L MilliQ H₂O (pH 7.8). This is sterilized and stored at room temperature.

PBS: PBS is used to perform washes of spheroids to rid of unbound phages and dyes. It is prepared by dissolving 1370 mM NaC1, 27 mM KC1, 100 mM of Na2HPO4, 18 mM of KH2PO4 in MilliQ H₂O (pH 7.4) and is filter-sterilized.

Bacterial and bacteriophage strains, plasmids and eukaryotic cells

TABLE 2
Summary of bacteria, phage plasmids and eukaryotic cells used in this study

| Cell | Genotype | Source/Reference | Additional Information |
|---|---|---|---|
| BB4 | supF58 supE44 | Agilent Technologies. Inc. | This strain encodes a |
| | hdR514 galK2 | | double suppression |
| | galT22 trp55 | | action (SupE and |
| | metB1 tonA | | SupF) and serves as |
| | DE(lac) U169 | | the positive control for |
| | | | the efficiency of plating |
| | | | (EOP) of λF7 samples. |
| W3101 | F-, galT22, λ-, | CGSC #4467 Bachmann | This strain does not |
| | IN(rrnD-rrnE)1, | (1972) | exert any amber |
| | rph-1 | | suppression action and |
| | | | serves as the negative |
| | | | control for λF7 |
| | | | samples as λF7 is not |
| | | | viable on this host. |
| W3101 | F-, galT22, λ-, | Nicastro et al. (2013) | Amber isogenic |
| SupD | IN(rrnD-rrnE)1, | | suppressor SupD |
| | rph-1, | | strain of W3101, |
| | uvrC279::Tn10, | | W3101 SupD |
| | serU132(AS). | | (serU132) |
| W3101 | F-, galT22, λ-, | Nicastro et al. (2013) | Amber isogenic |
| SupF | IN(rrnD-rrnE)1, | | suppressor SupF |
| | rph-1, | | strain of W3101, |
| | oppC506::Tn10, | | W3101 SupF |
| | tyrT5888(AS) | | (tyr75888) |

Phage Strains

| | | | |
|---|---|---|---|
| λF7 | λDam15imm21clts | Mikawa el al. (1996) | λ phage harbouring an |
| | | Maruyama et al. (1994) | amber mutation in the |
| | | | gene encoding gpD, |
| | | | and is able to form |
| | | | viable particles |
| | | | in the presence of the |
| | | | amber (UAG) |
| | | | stop codon at the |
| | | | position 68 of the |
| | | | 110 amino acid |
| | | | protein. |

Plasmids

| | | | |
|---|---|---|---|
| pPL451 | pM-c1857-pL- | Sokolenko et el. (2012), | Multicopy plasmid |
| gpD::EGF | c1857-pL-D::EGF- | Nicastro et al. (2013) | pPL451 that is under |
| (pD::EGF) | tL | | the control of a |
| | | | temperature-sensitive |
| | | | allele of the λ C1857 |
| | | | repressor. The EGF |
| | | | sequence is fused to |
| | | | the C-terminal of the |
| | | | gpD capsid gene that |
| | | | is separated by any in- |
| | | | frame linked sequence |
| | | | p (TSGSGSGSGSGT) |
| | | | SEQ ID NO:3 and a |
| | | | Kpnl cut site. |

Eukaryotic Cell Line obtained from the American Type Culture Collection (ATCC ®)

| | |
|---|---|
| NIH3T3 | Mouse fibroblast cells |
| HT29 | Human colorectal adenocarcinoma |

Cell culture: Cell lines are obtained from the American Type Culture Collection (ATCC®) and are maintained in T25 flasks at 37° C. in a humidified atmosphere of 95% air and 10% CO₂ Cells are cultured in Dulbecco's Modified Eagle's Medium (DMEM, Thermo Scientific) supplemented with 5% heat-inactivated foetal bovine serum (FBS). The culture medium is replaced every 3 d.

Spheroid culture: The characteristics and growth of multicellular spheroids with various seeding densities and cell lines are observed and recorded. At 90% confluency, NIH3T3 or HT29 monolayer cells are trypsinized (0.25% trypsin-EDTA) into single cell suspensions and are counted with a hematocytometer (Fisher Scientific). NIH3T3 fibroblast spheroids are generated at 30,000 cells for initial seeding density and are cultured for 10 d. HT29 colon adenocarcinoma spheroids are generated by seeding 5, 000 cells and are cultured for 7 d. To examine the spheroid growth over time, various seeding densities are imaged every 2 d using a bright-field microscope (Nikon® TE200). The spheroid diameters are measured using the QCapture Pro™ 7 software and Micropublwasher 5.0 RTV camera by projecting a bright-field image of the spheroid onto the screen and using the calibration wizard for the select objective lens and obtaining a measurement by extending the bar over the spheroid.

Spheroids are generated using 1.5% agarose (weight/volume) in serum-free DMEM and cast in a 96-well cell-culture plates. Cells are seeded in 200 11.1 of DMEM supplemented by 5% FBS and incubated at 37° C. and 10% CO₂. The growth of each spheroid is recorded for up to 14 d. Each spheroid is harvested using a glass pipette and treated with 10⁸ PFU of phage, unless otherwise stated.

Phage display: Phage display of EGF molecules on λ phage particles is achieved by the dual-control expression system that using amber mutant D capsid phage, λF7, that imparts a truncated gpD protein in a wildtype E. coli host. Through this system, viable progeny particles can be produced via amber suppression from suppressor strains of E. coli that possess mutated tRNA that reads the UAG stop codon and insert a different residue, allowing the full translation of Dam15 for a full length gpD capsid protein. W3101 SupD (serU132) and SupF (tyrT5888) are amber suppressor strains of E. coli that form alleles of gpD that is less optimal due to the insertion of amino acid serine and tyrosine, respectively, in the place of the UAG stop codon. In contrast, the SupE (gln1V44) suppressor strain counterpart can fully restore gpD by the replacement of glutamine in place of the amber stop codon, fully suppressing the amber mutation. Functional gpD alleles can also be provided in trans via complementation using a plasmid [pPL451 gpD::EGF] encoding a D allele translationally fused to the EGF or other gene of interest, resulting in the EGF peptides fused to gpD capsid proteins. Phage λ was selected for its ability to tolerate larger and higher density of protein fusions without compromising phage assembly.

Transformation: [pPL451-gpD::EGF] is a multicopy plasmid that expresses gpD:EGF and is under the control of a temperature-sensitive allele of the λ CI857 repressor that induces the gene at temperatures above 37° C., conferring ampicillin resistance to the host. A C-terminal EGF fusion to the capsid protein gpD is designed and constructed. Competent W3101 SupD and SupF—amber suppressor E. coli cells—are transformed with [pPL451-gpD::EGF] via heat shock and transformed colonies are selected on Luria Bertani (LB) ampicillin agar plates.

BB4 (supE, supF) E. coli is the double amber suppressor that results in functional λF7 phage particles without the presence of the fusion plasmid; this strain served as a positive control for efficiency of plating, and a negative control for EGF decoration (Nicastro et al., 2013).

Maximal decoration of λF7 is conferred by generating phages at higher temperatures (37° C.), using SupD E. coli amber suppressor host for propagation (denoted as ‘Hi-D::EGF phage’). Phages generated from the SupF E. coli strain resulted in reduced complementation from the plasmid expressing the gpD fusion than the SupE E. coli strain, therefore phages generated from SupF is used in this experiment to represent minimal surface decoration (denoted as lo-D::EGF phage’). Fluorimetric analysis of gpD::eGFP decorated phages in the same study estimated about 147 molecules per phage for the maximal fusion (SupD, 37° C.), and 89 molecules per phage generated from SupF at 37° C. From these values, it can be inferred that the Lo-D::EGF phages will be approximately 40% less decorated than Hi-D::EGF phages. These values are not absolute and have been provided as a point of reference to infer the relative degree of surface decoration.

Phage lysate preparation: Transformed SupD and SupF E. coli cells are grown in LB broth over night at 37° C. 10-fold dilutions of primary lysates are prepared in 1 ml of TN buffer and are added to 0.3 ml of the transformed cells (grown to OD600=0.4 at 37° C.), then incubated overnight at 37° C. 10 ml of TN buffer are added the next day on to each plate and incubated for 24 h at 4° C. The top agar is scraped and transferred to a conical tube, which is centrifuged at 23183×g at 4° C. for 20 min. Negative control λF7 generated with a double suppressor BB4 E. coli host (that will not confer phage display, denoted as wildtype phage herein) is prepared in parallel for future experiments. [0097] Phage purification: All phage lysates are filtered through a sterile 0.2 p.m syringe filter to remove cellular debris, followed by polyethylene glycol (PEG)-precipitation (Sigma-Aldrich). Precipitated phages are resuspended in DMEM supplemented with 5% FBS and are further purified through a gel chromatography. This is comparable to that purification via CsC1 centrifugation in dissociating unincorporated fusion and other smaller cellular debris. Phage samples are resuspended in DMEM and 5% FBS and standardized to a concentration of 10⁹ PFU/ml. 10011.1 of each sample is added to each spheroid for a total of 10⁸ PFU.

Phage titration and standard plaque forming unit assays: Plaque assays are used to determine concentration of phages following the purification process. It is also used to assess change in phage infectivity over time under various conditions. Soft-agar overlay technique is used on fresh BB4 E. coli cells, as this strain generates the highest titers of λF7 (it possesses double suppressor activity, SupE, SupF). A liquid culture of BB4 (300 μl) is added to 3 ml of molten top agar, as well as a 100 μl volume of phage dilution in suspension. Each phage particle produced a visible clearing zone on the lawn of bacteria; greater dilution of phages in the supernatant resulted in fewer plaques. Solutions containing purified phage are generally standardized to 10⁹ PFU/ml and 100 μl (10⁸ PFU) is added to each spheroid, unless otherwise stated.

Assessing phage viability: To test the infectivity of phages under the experimental conditions outlined, changes in phage titers are measured over time using the plaque assay. Wildtype and gpD::EGF expressing phages are incubated at both 4° C. and 37° C., suspended either in DMEM supplemented by 5% FBS or TN buffer, and are then measured at 4, 8, 24, and 48 h, post-application. Phages are quantified on E. coli BB4 cells and are plated on LB agar, diluted in TN buffer by a standard factor to generate countable plaques within the ranges of 20-200 PFU/plate. Efficiency of plating (EOP) is expressed relative to the initial phage titer determined at t=0 h as the 100% plating control.

EDTA sensitivity assay: Structural stability of phages displaying EGF molecules is tested using the EDTA sensitivity assay. Phages are diluted in TN buffer (0.01 M Tris-HCl and 0.1 M NaCl, pH 7.8; Fisher Scientific, USA) to a standardized concentration of 10⁸ PFU/ml, then are diluted 100-fold into TN/EDTA buffer (0.01 M Tris-HCl; (pH 7.8), 0.1 M NaCl, and 0.01 M EDTA) and incubated for 30 min at 25° C. The EDTA-phage reaction is stopped by diluting experimental reactions 100-fold into TN buffer. Phages are then plated on an overlay of BB4 cells and incubated overnight at 37° C. EOP is determined for each sample, using the formula below:

$$\frac{plaque counts by the original phage concentration}{\begin{matrix}100 \% positive plating control \\ ( using plaque counts on BB 4 cells )\end{matrix}}$$

Labeling of phage capsid for imaging: The primary amines of wildtype phage or EGF-displaying phage capsids are conjugated with Alexa Fluor® 488 (Thermo Fisher) according to manufacturer's instructions. Briefly, 10¹² PFU of PEG-precipitated phages are resuspended in 95 μl of 0.1-M NaHCO₃ buffer (pH 8.3) and are incubated with 5 μl of dye (10 mg/ml in dimethyl sulfoxide, (DMSO) at room temperature for 1 h with brief vortexing every 10 min. The volume is then brought up to 1 ml and is centrifuged for 30 min at 23183×g at 4° C., then for 10 min at the same speed. The unbound dye in the supernatant is replaced with DMEM and 5% FBS to resuspend the Alexa Fluor® 488-conjugated phages.

Confocal microscopy for phage visualization in spheroids: HT29 spheroids are grown for 7 d (seeding density of 5,000 cells). Spheroids are transferred into 1.5 ml microcentrifuge tubes and treated with 10⁸ PFU of phage for either 30 min, 4 h, 8 h and 24 h and incubated at 37° C., 10% CO₂. Each spheroid is stained with 10 μM of CellTracker Red CMPTX dye™ (Molecular Probes, Invitrogen) for 1 h at 37° C. prior to visualization. CellTracker is mildly thiol-reactive and can diffuse through the live cell membranes. It is retained in the cell for at least 24 h as it becomes membrane-impermeable by reacting with other intracellular proteins. Cell medium is discarded, and spheroids are washed 3× with PBS and transferred with a glass pipette onto a glass bottomed 24-well plate (MatTek P-24G-0-13 F). Treated spheroids are visualized with laser scanning confocal microscope, 20× objective (Zeiss LSM 710). Z-stack images from the bottom to the center are obtained for each spheroid.

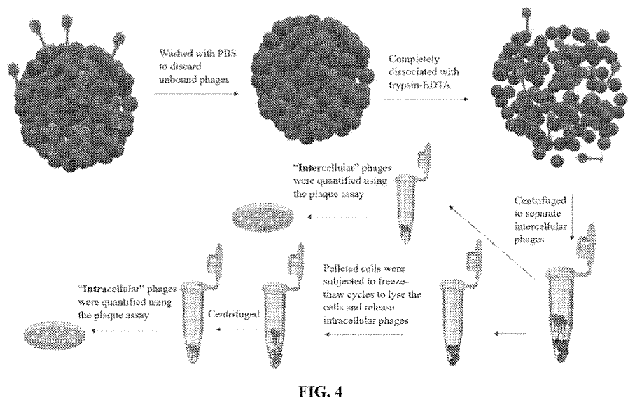

Internalization assay: HT29 spheroids are grown for 7 d (seeding density of −5,000 cells). Prior to the application, spheroids are treated with 5 mM of NH₄Cl for 45 min at 23° C. to prevent internalized phages from entering the lysosomes. Ammonium chloride and chloroquine are considered to be lysosomotropic weak bases that selectively accumulate in lysosomes to reduce endosome-lysosome acidification. Spheroids are washed with PBS and treated with 10⁸ PFU of phages for 30 min, 4 h, 8 h and 24 h at 37° C. Following the incubation periods, spheroids are washed 5 times with PBS to remove unbound phages. Cold 0.2 M glycine (pH 2) is added to spheroids for 10 min, followed by 2 washes with PBS to inactivate unassociated phages suspended in the media. 100 μl of 0.25% EDTA-trypsin is added to spheroids and incubated at 37° C. for 20 min, then cells are vigorously pipetted and gently vortexed for complete disaggregation. The suspended cells in trypsin are centrifuged at 7,000×g for 10 min to separate the phages recovered from the intercellular spaces of the spheroids. The supernatant is removed and set aside as “intercellular” phages for each sample (FIG. 4, showing that spheroids are pre-treated with ammonium chloride prior to the addition of phages for 0.5, 4, 8 and 24 hours. Spheroids are washed with PBS to discard unbound phages on the surface of spheroids. Spheroids are then completely dissociated with trypsin-EDTA to release intercellular phages within the spheroid. Cells are lysed by freeze-thawing, which released intracellular phages. All recovered phages are quantified using the plaque assay). The pelleted cells are resuspended and washed in PBS, and the centrifugation step is repeated. The wash media is collected in the same “intercellular” fraction. The pelleted cells are resuspended in 100 pi MilliQ H2O, subjected to 3 freeze/thaw cycles to release internalized phage particles. This mixture is centrifuged and the supernatant is set aside as the “intracellular” fraction of the phage mixture. The collected fractions are serially-diluted by 10⁻²-10⁻⁸ in PBS, and 10 μl of each dilution of phages are “spotted” onto a lawn of BB4 cells to determine the optimal dilution factor for well-separated, countable plaques. These plates are incubated overnight at 37° C. and whole plates are prepared the next day to determine the EOP of each fraction.

Phage λ and effects on spheroid growth: 5,000 HT29 cells are seeded on agarose to form spheroids previously described. Phages are added to partially-formed and fully-formed HT29 cells; cells are also co-seeded with phages to observe the effects of phages on initial cell-aggregation. The following conditions are imaged with brightfield illumination on a Leica DM2000 microscope, 10× objective and a QImaging Micropublwasher 5.0 RTV camera using the QCapture Pro™ 7 Software (QImaging). No phages are added to the “control spheroid”. All images are qualitatively representative of three replicates of two independent experiments:
i. Cells are grown for 48 h on agarose for initial aggregation for partially-formed spheroids prior to the addition of 10⁸ or 10⁴ PFU of resuspended D::EGF or wildtype phages. Pictures are obtained every 2 d over a duration of 20 d.
ii. Cells are co-seeded with 10⁸ or 10⁴ PFU of D::EGF or wildtype phages and are grown for 7 d. Pictures are obtained every 2 d over a duration of 7 d.
iii. Cells are pre-chilled with 10⁸ or 10⁴ PFU of D::EGF or wildtype phages for 1 h on ice, then co-seeded on to agarose. Pictures are obtained over a duration of 10 d.
iv. Fully-formed HT29 spheroids, cultured for 5 d (seeded at 3,000 cells), are treated with 10⁸ or 10⁴ PFU of D::EGF or wildtype phages. Pictures are obtained over a duration of 5 d.

MTT assay: The size of the viable cell population with the treatment of phages is measured by the 3-(4,5-dimethylthiazol-2-yl)-2,5-diphenyltetrazolium bromide (MTT) assay and its conversion to formazan by viable cells. Cells are seeded in DMEM supplemented with 5% FBS in 96-well plates and incubated at 37° C., 10% CO₂ for 48 h to allow for initial adherence. Cells are then treated with either 10⁸ or 10⁴ PFU of D::EGF or wildtype phages for 24 h at 37° C., 10% CO₂. The medium is aspirated and 20 μl of 0.5 mg/ml MTT in PBS is applied and the plate is swirled and incubated at 37° C. for 1 h to allow for the conversion of MTT into formazan. The MTT reagent is then removed and replaced with 100 pi of DMSO per well for 30 min at room temperature for the dissolution of formazan dye. At the end of this incubation period, the absorbance of the plate is measured at A492 nm on a SpectraMax M5 Plate Reader. The results are normalized by subtracting A492 of blank wells.

3D Toxicity assay: Phage-treated HT29 spheroids are assessed for cytotoxicity using CellTiter-Glo® 3D (Promega)—a viability assay for 3D cell cultures. It determines the number of viable cells in multicellular spheroids based on the quantitation of ATP, a marker for metabolically-active cells. This assay is used to visually determine whether cells within spheroids remained viable in response to phage treatment. This is conducted according to the manufacturer's directions, as follows. 5,000 HT29 cells are seeded to produce robust spheroids, as previously described, and grown for 7 d. Spheroids are transferred with glass pipettes and placed into opaque multiwell plates. A volume of acid solution (4 mM diaminocyclohexane triacetic acid in water) is added, equivalent in volume to the cell culture medium containing the spheroid. The contents are mixed vigorously for 5 min on an orbital shaker and incubated at room temperature for additional 15 min. Half of the sample is transferred to a separate container and diluted by 10-fold in MilliQ H2O with repeated mixing to ensure homogeneity. The diluted sample is then transferred to a fresh assay well. An equal volume of CellTiter-Glo® 3D Reagent is added, and the contents are mixed for an additional 2 min on an orbital shaker. The plates are incubated at room temperature for 10 min to stabilize the luminescent signal, which is then recorded on the SpectraMax M5 spectrophotometer using the luminescence mode at the wavelength range of 250-850 nm.

Spheroid embedding in paraffin: HT29 spheroids grown for 7 d (seeding density of 5,000 cells) are treated with fluorescently-tagged wildtype or D::EGF phages. The spheroids are placed in 20× volume of 10% formalin at room temperature and dehydrated by passing through successive changes of 70% ethanol (in Millipore water), two changes in 100% ethanol and two changes in 100% xylene. The spheroids are embedded into paraffin wax by passing through two changes of 100% wax at 56-58° C. The sections are mounted onto slides and air dried. The sections are deparaffinized the following day in 2-3 changes of xylene, 5 min each. Sections are then hydrated in 2 changes of 100% ethanol for 3 min each, 70% ethanol for 1 min each. Samples are fully hydrated with 2 changes of MilliQ H2O and mounted with aqueous mountant (Biomeda©).

Statistical analysis: GraphPad Prism 5 software is used for the analysis of data and a threshold of p<0.05 is deemed to be significant for all statistical tests. Changes in phage viability in response to temperature is analyzed by two-way analysis of variation (ANOVA) by comparing values between incubations at 4° C. and 37° C. HT29 spheroids treated with ammonium chloride is compared with the untreated by the Mann-Whitney test. Percent internalization of D::EGF phages are compared to that of wildtype for each treatment duration by the Mann-Whitney test. Percent viability of HT29 cells in response to phage treatment is compared to the untreated cells by the Mann-Whitney test. Experiments are performed with a minimum of three independent biological replicates. *p<0.05, ″p<0.005, ****p<0.005

### EXAMPLES

Bacteriophages have emerged as potent and efficient tools in medicine, specifically with the advent of phage display technology. This technology has created new avenues for phages in cancer treatment as a means for targeted delivery into malignant cells and tissues in recent years. Their genetic flexibility to undergo surface modifications to target specific cells and tissue types have made them an attractive target in therapeutics. Here, the inventors discovered that λ phages naturally adhere to the surface of mammalian (mouse and human) cells and can accumulate in three-dimensional multicellular spheroids, in which both stromal and neoplastic cells of a tumour could be modeled.

The inventors noted that display of peptides to the surface of phages can enhance specific targeting of phages to cells and tissues. EGF has been successfully used for targeting of other types of vectors in several gene therapy applications to date. EGFR blockade has also been implicated in slowing of tumourigenesis and cancer progression. HT29 colon adenocarcinoma cells were selected for spheroid culture, which expresses high EGFR levels. The inventors noted that multivalent EGFR display on phages would likely enhance their accumulation in HT29 spheroids.

Therefore, the inventors contemplated that bacteriophage Lambda (λ) can interact with multicellular spheroids generated from colorectal adenocarcinoma HT29 cells and mouse fibroblast NIH3T3 cells. The display of EGF on lambda phage enhanced binding, uptake and accumulation of the phage into the tumour microenvironment of the spheroids and internalization within cells of the tumour microenvironment. The specific binding to EGF receptors on cells can thereby slow spheroid formation and growth.

HT29, which expresses the highest level of EGFR in relation to other colorectal cancer cell lines, was selected to determine whether tumour growth was altered by the signal intervention with D::EGF phages. They are also representative of wildtype-KRAS and BRAF-mutated tumours, which have been reported in approximately 5-15% of patients with colorectal cancers. EGF was the peptide of choice for display, as it retains its function when displayed on phage, and has been well-documented in targeting of other types of vectors in gene therapy applications.

The inventors found that the bacteriophage has a potential as a tumour nanocarrier system. In the examples following, multicellular spheroids are used to model the tightly-packed tumour microenvironment and responses to the application of λ phages are described. Phages displaying ligands for overexpressed receptors are also examined in their accumulation in spheroids. In detail, the inventors used NIH3T3 fibroblast and HT29 colon adenocarcinoma multicellular spheroids to represent the stroma and parenchyma of solid tumours and then to examine infiltration of bacteriophages as a means of delivering therapeutic cargo. By using phage display technology to decorate the surface of bacteriophage with epidermal growth factor (EGF), the inventors compared the cell-phage interactions between wildtype phages and EGF-displaying phages, including the assessment of phages' capacity to traverse the tumour interstitium, to be subjected to cell internalization and their effects on spheroid growth. Both wildtype and EGF-displaying phages are observed to adhere to the HT29 and NIH3T3 spheroids and to internalize into cells as early as 30 min following administration. EGF-phage treatment also slowed HT29 spheroid growth—demonstrating a delay in initial aggregation and formation of loosely-organized structures that led to much smaller spheroid formation in the latter stages of growth. These results support the potential for the therapeutic employment of bacteriophages as nanocarriers for targeted delivery.

### Example 1

### Characterization of Spheroids with Different Seeding Densities

Cells were seeded on agarose, which resists cell adhesion and causes the cells to preferentially stick to each other and form spheroids. The growth of spheroids and morphological changes were monitored for the following cell lines: HT29, MDA-MB-231, A549 and A2780. Growth of spheroids were visualized with a bright-field microscope for up to 10 d to choose optimal cell lines for downstream applications.

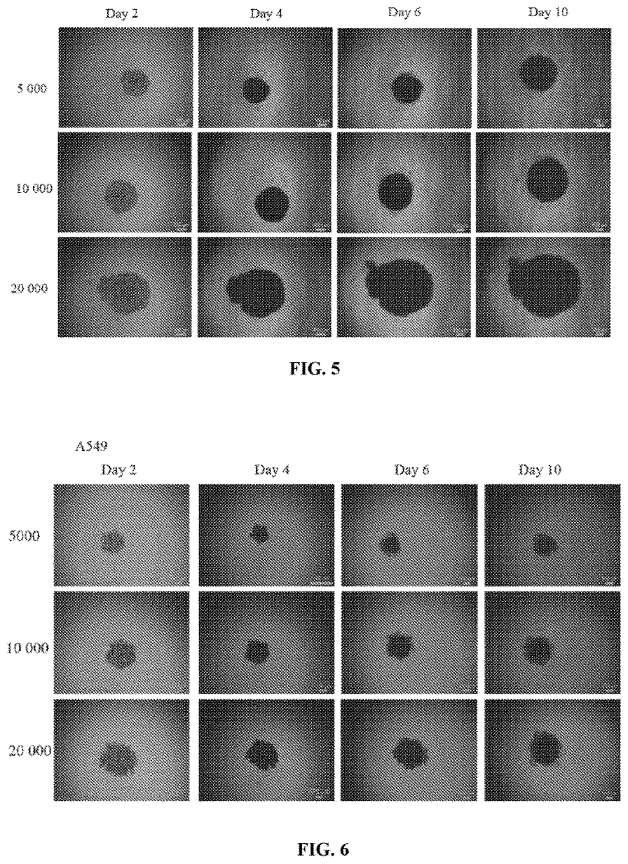

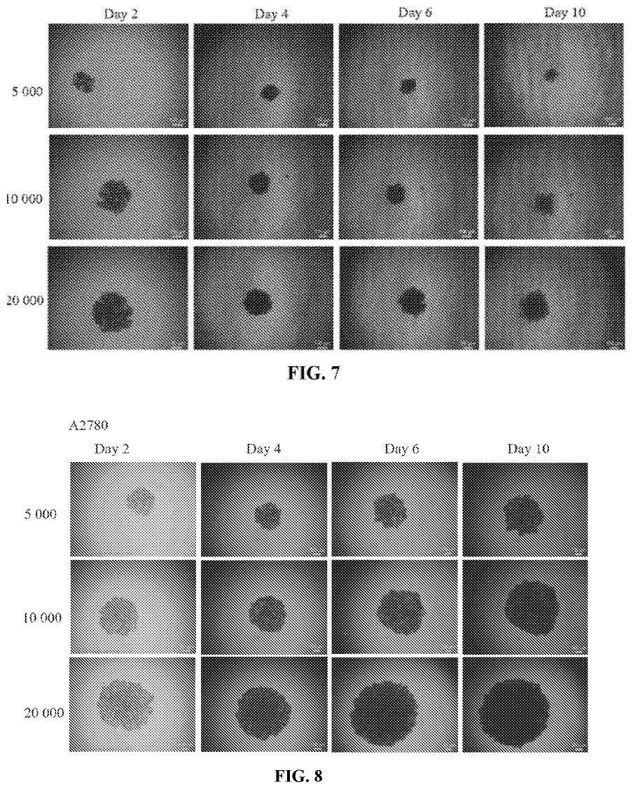

FIGS. 5-8 show representative optical images of spheroids with different cell seeding densities. Several stages were uniformly observed in spheroid formation across most cell lines; within the initial 24 h after seeding, cells spontaneously aggregated and appeared to form of intercellular connections. However, MDA-MB-231, A549 and A2780 spheroids were not robust and disintegrated upon handling. A2780 spheroids in particular, showed dispersed cells with visible intercellular gaps after 10 d, likely having failed in forming effective cell-cell connections (FIG. 8 showing growth of A2780 spheroids at various seeding concentrations, imaged for 10 d. Cell-cell connections appeared to be loose and gaps were visible between cells. Spheroids did not form fully and appeared to be disc-like in morphology). Conversely, robustness was achieved within 6-10 d of growth for HT29 cells seeded at a concentration of 5,000 to 30,000 cells (FIG. 5, showing Growth of HT29 spheroids at various seeding concentrations, imaged for 10 d. Spheroids seeded with more than 10,000 cells were generally flatter in morphology. These cells dispersed laterally onto agarose, which could not be used to assess phage penetration. 6-10 day-old spheroids generated from 5,000 cells were used in all experiments, unless otherwise stated.). Definite intercellular connections appeared to have established by days 3-5, as the surfaces of spheroids were smoother and diameters decreased slightly. This was likely a result of cells adhering more tightly to each other. Diameters increased again by day 6, and cell proliferation was visible on days 4-6. Spheroids exhibited rougher edges and cells scattered in the surrounding media. Cells visibly dissociated from spheroids on day 10, as cells lost their circular morphology. The relationship between cell seeding concentrations and spheroid diameters for cell lines HT29, NIH3T3 and HCT116 after 5 d of growth are shown in Table 3. FIG. 6 shows growth of A549 spheroids at various seeding concentrations, imaged for 10 d. Spheroids demonstrated poor reproducibility and were also generally flatter in morphology. FIG. 7 shows Growth of MDA-MB-231 spheroids at various seeding concentrations, imaged for 10 d. Spheroid morphology was inconsistent among replicates and demonstrated poor reproducibility.

TABLE 3
HT29, NIH3T3 and HCT116 spheroid diameters are
dependent on initial cell-seeding densities

| | Diameter of | Diameter of | Diameter of |
|---|---|---|---|
| Seeding | NIH3T3 spheroids | HT29 spheroids | HCT116 spheroids |
| Densities | (μm)¹ | (μm)¹ | (μm)¹ |
| 100 | N/A² | 70 ± 8.2 | N/A |
| 500 | N/A | 175 ± 19.2 | 150 ± 18.2 |
| 1,000 | N/A | 205 ± 20.4 | 175 ± 19.7 |
| 5,000 | 70 ± 10.5 | 320 ± 35.1 | 225 ± 28.3 |
| 10 000 | 130 ± 15.9 | 580 ± 50.3 | 530 ± 41.2 |
| 20 000 | 190 ± 16.2 | 700 ± 66.4 | 615 ± 50.8 |

¹Diameters are represented as mean +/− SEM of 3 separate spheroids
²N/A denotes the insufficient cell density for effective aggregation and ability to form a defined spheroid
Diameters of each group were highly uniform (SD < 15%)

HT29 cells were selected for downstream applications because diameters were uniform and reproducible—90% of wells formed spheroids and diameters varied by less than 15% (Table 3). Reproducibility of spheroid sizes was crucial to ensure consistency in parameters that could ultimately influence the pharmacokinetics of phage penetration. However, it is important to note that individual spheroids can still be highly heterogeneous despite exhibiting similar diameters. HT29 cells can also be useful in modeling epithelial cells forming tight junctions between cells. In this project, −350 μm diameter HT29 spheroids, initially seeded with 5,000 cells and grown for 6-10 d were often used for treatment. Larger spheroids may exhibit hypoxic and necrotic cores, which may potentially complicate the characterization of phage penetration because of accumulated fluid and cellular debris.

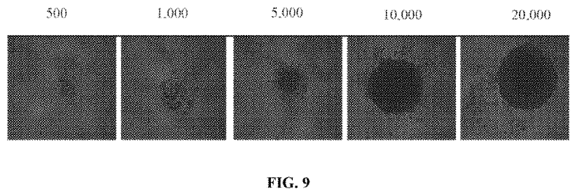

The complete aggregation of cells to form robust spheroids was also contingent on the initial seeding densities. For example, the initial concentration of <1,000 cells was not sufficient for aggregation after 7 d and failed to form defined spheroids (FIG. 9, showing that HT29 cells seeded at various concentrations after 7 d. Cell proliferation was evident by the scattered cells surrounding the spheroids. Seeding fewer than 1,000 cells resulted in aggregation of cells, but they failed to form defined spheroids). HT29 spheroids seeded with >10,000 cells were compact and usable for downstream applications.

### Example 2

### Generation of EGF-Displaying Phages and Packaging Tolerance of gpD::EGF Fusion

The ability of the pD::EGF plasmids to complement the Dam15 mutation of λF7 to generate phages displaying EGF was dependent on the tolerance of the gpD allele conferred from the SupD and SupF isogenic suppressor strains. Theoretically, poor suppression of the amber mutation by the Sup+ hosts would lead to the greater reliance on the pD::EGF plasmids for complementation of gpD. This was determined by assessing the restoration of viability of λDam15 phages generated from the different Sup+ hosts as compared to the Sup− wild type E. coli host.

TABLE 4
pD::EGF complements the Dam15 mutation and increases viability
of λF7 phages in the absence of suppressor host E. coli

| | Relative efficiency of | Increase in plating |
|---|---|---|
| Strain [+/− Plasmid]¹ | plating (EOP)² | (compared to Sup−) |
| Sup− | 28 × 10⁻⁵ | 1.0 |
| Sup− [pD::EGF} | 0.05 | 1.79 × 10³ |
| SupD | 1.6 × 10⁻⁵ | 0.57 |
| SupD [pD::EGF] | 0.12 | 4.29 × 10³ |
| SupF | 0.23 | 8.21 × 10³ |
| SupF [pD::EGF} | 0.10 | 3.57 × 10³ |

¹The E. coli suppressors are W3101 derivatives, and the plasmid pPL451 is under the temperature-regulated repressor C1857.
²All EOPs have been expressed as a mean of three assays with 3 replicates, determined using BB4 (SupE, SupF double suppressor E. coli) as the 100% plating control, plated at 37° C.

Table 4 shows the ability of the gpD::EGF expression plasmid to complement for the Dam15 mutation of λF7 by restoring plating by more than a 1000-fold in the absence of suppressor activity (Sup−). The EOP (determined by BB4, SupE, SupF double suppressor, as the 100% plating control) increased by over a 4000-fold in SupD with the gpD::EGF complementation despite the poor suppressor activity and low viability on the SupD derivative. SupF strain possesses effective suppressor activity and there were no significant differences with the complementation for the Dam15 mutation on the viability of phage. These results were in agreement with those of Nicastro et al. 2013, whereby the SupD host was the least effective at reversing the Dam15 mutation, while SupF suppressed the mutation with near perfect efficiency. Plasmid pPL451 is under the control of a temperature-sensitive repressor, CI857, but all phages were generated at 37° C., rendering the repressor ineffective and allowing the expression of downstream gene sequences. This resulted in maximal fusion of EGF to gpD on the λDam15 phage capsid at 37° C., specifically in the suppressor host with the poorest functionality, SupD E. coli.

Assessment of phage viability in response to temperature: Phages are typically stored at 4° C., which generally provides long-term stability (Lech, Reddy, & Sherman, 1990). The effects of longterm incubation at 37° C. and in cell media on the ability of wildtype and EGF-displaying phages were assessed. Phages were exposed to these conditions for defined intervals up to 48 h and plaque assays were used to quantify changes in the concentration of infective phage particles over time.

TABLE 5
Prolonged exposure of phages at 37° C. decreases λ phage infectivity

| | | | | EOP of | EOP of |
|---|---|---|---|---|---|
| | | EOP of | EOP of | D::EGF | D::EGF |
| | | λF7 | λF7 | phages | phages |
| | | incubated at | incubated at | incubated at | incubated at |
| | | 4° C.¹ | 37° C.¹ | 4° C.¹ | 37° C.¹ |
| 0 | h | 1.00 ± 0.02 | 1.00 ± 0.05 | 1.00 ± 0.05 | 1.00 ± 0.02 |
| 4 | h | 0.98 ± 0.01 | 0.91 ± 0.01* | 0.95 ± 0.04 | 0.57 ± 0.02**** |
| 8 | h | 0.86 ± 0.05 | 0.73 ± 0.02*** | 0.82 ± 0.05 | 0.43 ± 0.04**** |
| 24 | h | 1.12 ± 0.02 | 0.46 ± 0.02**** | 0.86 ± 0.03 | 0.19 ± 0.08**** |
| 48 | h | 0.94 ± 0.03 | 0.35 ± 0.03**** | 0.86 ± 0.02 | 0.28 ± 0.04**** |

¹All EOPs have been expressed as a mean ± standard deviation of three independent assays, n = 3, determined using B4(SupE, SupF double suppressor) as the 100% plating control and plated at 37° C.
Comparisons of each phage types at two temperatures were made for each treatment period using the 2-way ANOVA test. Viability decreases of both phage types at 37° C. were statistically significant for all treatment periods.

This viability assay provides a relative change in phage titers; it is not an accurate representation of total phage particle counts in the media. Re-suspension and dilution of phage incubations in cell media supplemented at 4° C. slightly reduced phage infectivity, but this effect on phage viability increased dramatically at 37° C. Changes in phage viability for both phage types with higher temperatures were statistically significant at all treatment durations, but D::EGF phages were impacted more by these external conditions than the wildtype λF7 phages. To minimize the effects of external variables on experimental outcomes, all titer assays were expressed as EOPs to the PFU/ml of the phage concentrations at each set of conditions in Table 4 to accommodate for these confounding variables. All experiments were conducted with maximum incubation periods extending up to 24 h at 37° C., as phage titers were decreased dramatically by this time point.

EDTA resistance by a, wildtype and λD::EGF phages: Phages expressing EGF decoration were assessed for structural integrity with incubation in EDTA. EDTA is a chelating agent and will sequester cations such as Mg⁺ and Ca⁺, which are necessary co-factors to destabilize the λ capsid.

TABLE 6
Susceptibility of Display Phage to EDTA

| | Relative efficiency |
|---|---|
| Strain [+Plasmid] | of plating (EOP)¹ |
| SupD [pD] | 0.41 ± 0.03 |
| SupD [pD::EGF} | 0.62 ± 0.02 |
| SupF [pD] | 0.46 ± 0.06 |
| SupF [pD:EGF] | 0.61 ± 0.04 |
| Sup− [pD:EGF} | 0.21 ± 0.05 |
| Sup− [pD] | 0.89 ± 0.03 |

Phages grown on SupD E. coli had a lower survival rate (41%) than lysates grown on SupF (46%), which may be attributed to the higher stability in packaging. Decoration of the gpD protein resulted in consistent survival rates of 62% and 61% for the maximal decoration and minimal decoration, respectively. Phages developed on Sup− [pD::EGF] exhibited low survival rates of 21%, which may indicate low tolerance for the incorporation of the D::EGF into the phage capsid. Combination of complementation with pD::EGF and gpD alleles was found to be more stable than either one alone. Sup-[pD] conferring the wildtype gpD allele had the greatest resistance to EDTA at 89%, as expected.

### Example 3

### Bacteriophage λ Infiltration of Multicellular Spheroids

To demonstrate λ association and infiltration into the spheroids, 3 μm cross-sectional Z-stack images of spheroids were obtained with an inverted confocal microscope. The inner, densely-packed regions of the spheroid could not be observed, where the scattering of light obscured the detection of fluorescent phages in these regions. General observations were made on the spheroid surface-phage interactions as well as the overall accumulation of phages in the visible regions of the spheroid.

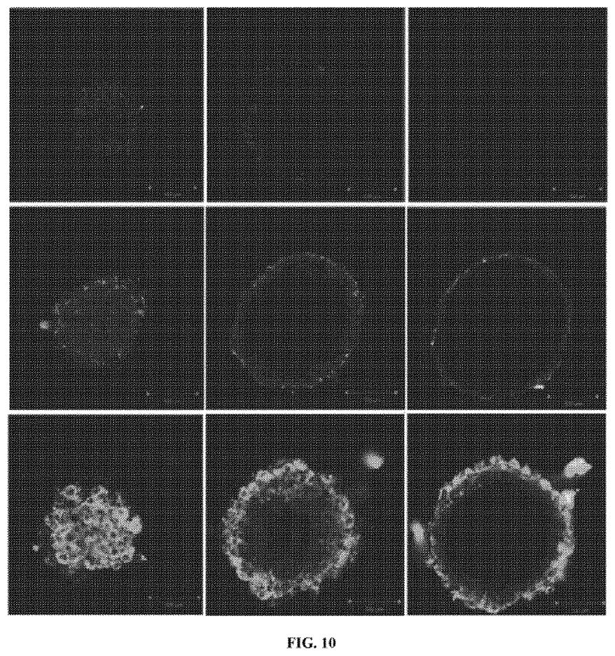

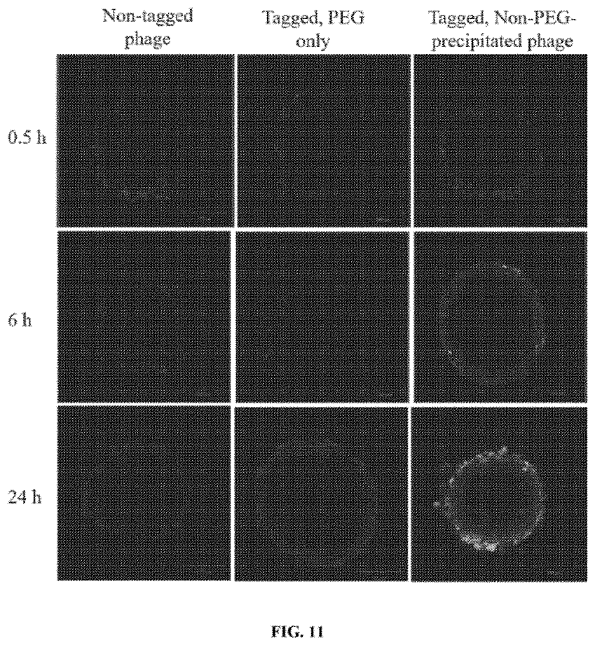

Interaction of λ phages with NIH3T3 spheroids: First, in order to observe the native interaction between unmodified, wildtype phages and fibroblast spheroids, 10⁸ PFU of wildtype XF7 phages tagged with Alexa Fluor® 488 were added to each 6-d old NIH3T3 spheroid for 0.5, 6 and 24 h. Spheroids were washed to discard unbound phages and were stained with CellTracker (red) (FIG. 10). After 6 h of treatment, the phage-associated fluorescence (green) was concentrated primarily at the periphery of the spheroid and were visible in the spheroid depths of 25 p.m. After 24 h of phage treatment, phage-associated fluorescence completely dominated all cross-sections of the spheroid, likely indicating that phages have successfully accumulated in the spheroid (FIG. 10, showing Optical cross-sections of NIH3T3 spheroids treated with fluorescent λ phages. Representative images of spheroids treated for A. 30 min, B. 6 h, and C. 24 h at optical sections 25, 55 and 90 p.m from the bottom-most surface (n=3). All the spheroids were counterstained with CellTracker™ Red prior to imaging. Green fluorescence is associated with the presence of Alexa Fluor 488-phages.

The purification process of phages involved precipitation of phages by polyethylene glycol (PEG). This raised the possibility of PEG being bound by Alexa Fluor 488 and providing false positive signals in FIG. 10. Therefore, PEG was processed with Alexa Fluor 488 as a control, without the addition of phages, in FIG. 11 (showing NIH3T3 spheroid treated with polyethylene glycol.

Representative images of NIH3T3 spheroids treated with A: unlabeled X. phages, B: polyethylene glycol (PEG) without the addition of phages subjected to Alexa Fluor 488 reaction; and C: Alexa Fluor 488-X. phages. Spheroids were treated for 0.5, 6 and 24 h. Optical sections 55 μm from the bottom of spheroids are shown. All the spheroids were counterstained with CellTracker™ Red prior to imaging. Green fluorescence is associated with the presence of Alexa Fluor 488-phages. All images are representative of three replicates of spheroids.

The very low signal in the PEG-only control in FIG. 11 suggests the view that the strong green fluorescence observed in FIG. 10 was very likely emitted by tagged phages. It can be inferred that phages began to adhere to the surface of the spheroid as early as 0.5 h and diffused through the intercellular spaces, freeing up the external surface of cells at 6 h. More phages bound to the external surface of spheroids and accumulated in a time-dependent manner up to 24 h (FIG. 10). Based on these results, there appears to be a great level of natural interaction between unmodified, wildtype phages and NIH3T3 fibroblasts, and infiltration into NIH3T3 spheroids likely occurs as early as 6 h after 10⁸ PFU of phage application.

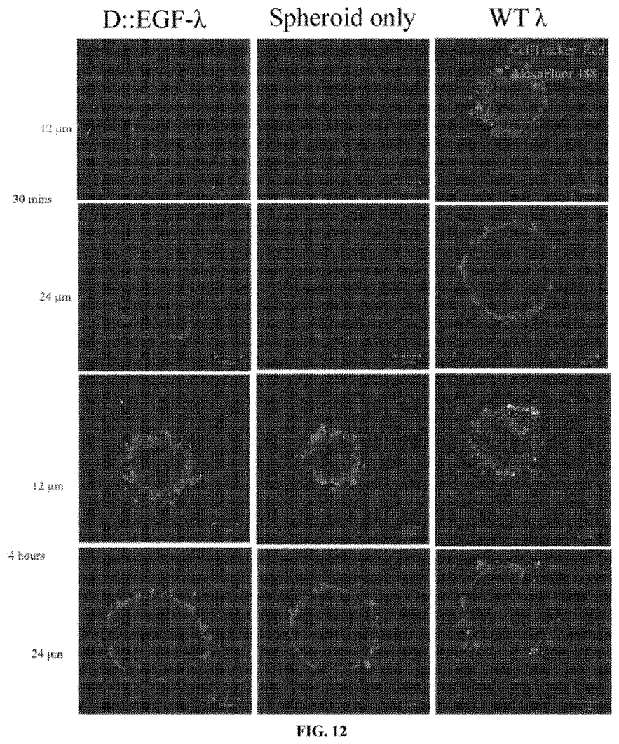

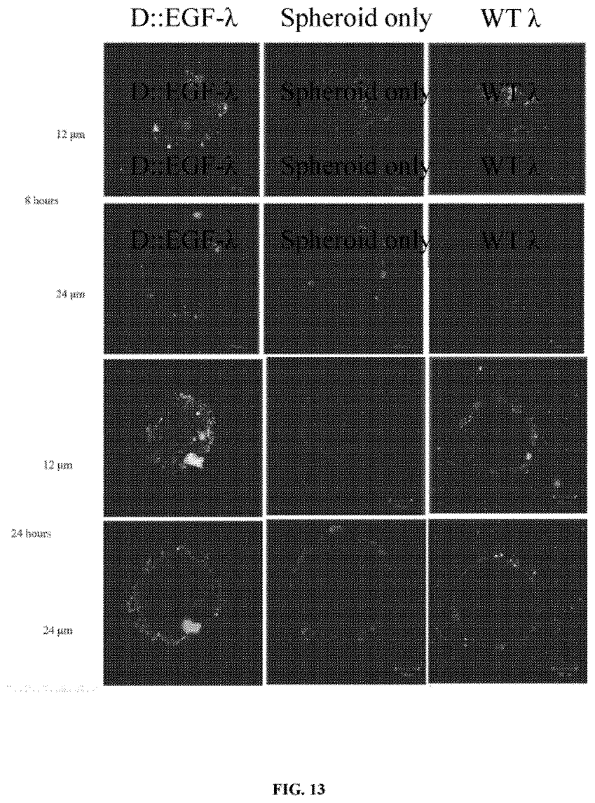

Interaction of λD::EGF phages with HT29 spheroids: Spheroids were generated from HT29 cells to study phage accumulation in the context of rapidly proliferating neoplastic cells that form tight cell-cell contacts. EGFR is overexpressed in HT29 cells and has been associated with aggressive biological behaviour. D::EGF phages (EGF fused to gpD capsid proteins) were generated for maximal fusion of molecules (grow on the SupD host at 37° C.). 10⁸ PFU of either the wildtype and D::EGF phages were added to HT29 spheroids (−300 p.m diameter) for up to 24 h, and Z-stack images were obtained at 12 and 24 μm depth from the bottom surfaces (FIG. 12, 13). In detail, FIG. 12 shows Optical cross-sections of HT29 spheroids treated with wildtype phages and EGF-displaying phages for 30 min (top) and 4 h (bottom). 6-d old HT29 spheroids (−300 μm diameter, seeded with 5,000 cells) treated with Alexa Fluor 488 tagged phages (green) for given durations. Z-stacks were obtained at 12 and 24 μm from the bottom-most surface of the spheroids. All images are qualitatively representative of three replicates of spheroids. FIG. 13 shows representative cross-sections of HT29 spheroids treated with wildtype phages and EGF-displaying phages for 8 h (top) and 24 h (bottom), which is continued from FIG. 9.

Fluorescent wildtype (EGF-)phages were visible on the outer edges of the surface of HT29 spheroid as early as 4 h. Spheroids treated with D::EGF phages were relatively free of fluorescence until after 8 h of treatment. After 24 h, positive fluorescence was visible in the spheroids treated with both types of phages at 12 and 24 μm depths. Positive fluorescent spots were observed throughout all optical sections.

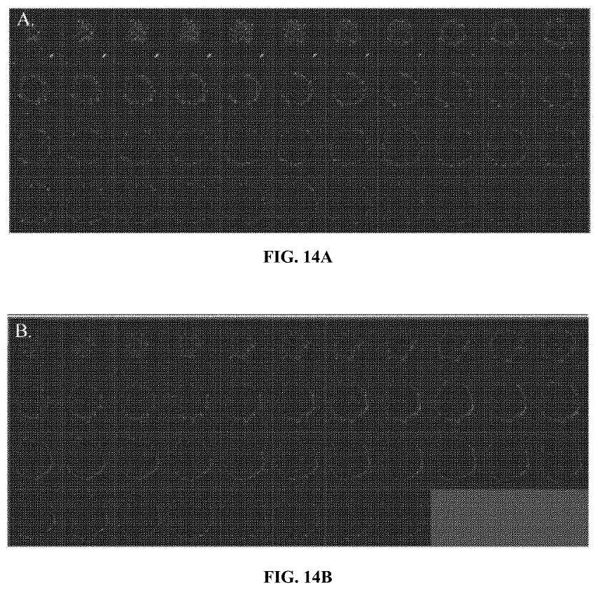

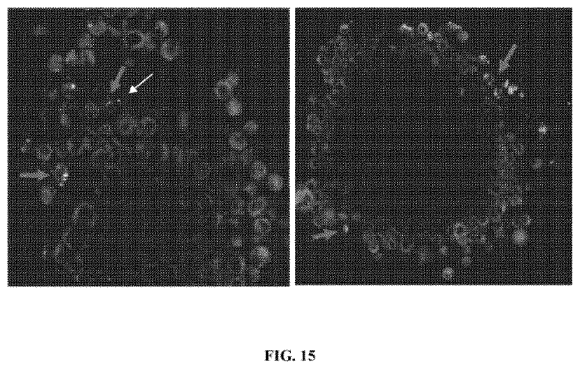

FIG. 14A and FIG. 14B show the collective Z-stacks of HT29 spheroids treated with D::EGF phages (FIG. 14A) and wildtype phages (FIG. 14B) after 24 h. Each square represents 3 μm slices of spheroids from the bottom-most surface (top left) to the centre (bottom right). 6-day old HT29 spheroids (−300 μm diameter, seeding concentration of 5,000 cells) were washed to discard unassociated phages (green) after incubation periods. There were noticeably more green fluorescent spots embedded in the spheroid treated with λ D::EGF phages than with wildtype phages, in all optical sections. There also appeared to be more fluorescent particles floating in the medium of the spheroid treated with D::EGF phages than the WT phages (FIG. 14A & FIG. B, respectively, top left squares). This may have been a result of more loosely-bound phage particles on the spheroid coming unbound and settling to the surface, after spheroids were transferred.

### Example 4

### Bacteriophage Internalization by HT29 Cells

FIG. 15 shows phage-associated fluorescence emitted inter- and intracellularly of HT29 cells. HT29 spheroids were treated with wildtype phages (left) and EGF-displaying phages (right) for 4 h. Certain spots of positive green fluorescence exhibited diffuse edges, appearing to be confined by the boundaries of the cell membranes (arrows, FIG. 15). This suggested the possibility that they were emitted from within cells, rather than from the surface of the cells. Intracellular fluorescence suggests that the phage-associated particles have been internalized by HT29 cells. The inventors queried other studies involving the use of confocal imaging to visualize cells stained with CellTracker to use as a point of reference for the appearance of these internalized, fluorescent particles. β3 integrin antibodies labeled with Alexa Fluor 488 internalized by cells stained with CellTracker Red. Once internalized, these antibodies also appeared yellow with diffuse edges. The inventors determined that the extracellular fluorescence would appear more discrete with distinct edges, such as those indicated by white arrows in FIG. 15.

Quantitation of internalized bacteriophage a, particles: To further investigate the potential cellular uptake of phages by HT29 cells, cells comprising the spheroids were lysed to release internal contents and the PFU was quantified by a conventional plaque assay. Previous studies have shown that recovered intracellular phages can retain their infectivity after 24 h. However, as phages are not stable at 37° C. outside of a bacterial host, this temperature was expected to reduce overall viability counts (Table 5). Additionally, to investigate the potential D::EGF-EGFR association, λD::EGF phage decorated with fewer molecules, approximately 40% fewer per phage (Lo-D::EGF phage) than the achievable maximal decoration (Hi-D::EGF phage), was generated using the SupF E. coli host. The purpose of generating a less-decorated phages was to determine whether the phage-cell association would be reduced in response to the decreased avidity of the presumed D::EGF-EGFR interaction. These phages were also fluorescently tagged and added to HT29 spheroids similarly as above, alongside the Hi-D::EGF phages and wildtype phages.

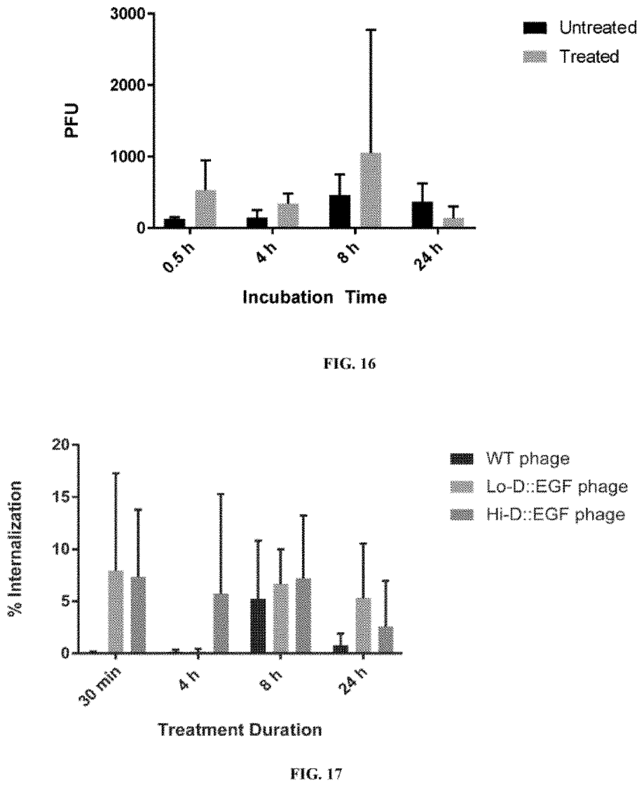

In order to minimize intracellular trafficking of phage particles into lysosomes, spheroids were pre-treated with a lysosomotropic agent (ammonium chloride) prior to the addition of phages. This was expected to minimize the degradation of phage particles that would underestimate the overall quantitation of internalized phage particles, First, the effects of ammonium chloride in improving phage viability was assessed by comparing the enumeration of wildtype phage recovered from pre-treated spheroids to untreated spheroids (FIG. 16). Cells were lysed using the freeze-thaw method, and in order to fully recover internalized phages by cells situated closer to the core, spheroids were completely dissociated by trypsinization at 37° C. FIG. 16 shows testing the action of ammonium chloride as a lysosomotropic agent to improve phage recovery. 6-d old HT29 cells (seeding density of 5,000 cells) were treated with 5 mM of ammonium chloride for 45 min at room temperature. Following treatment, remaining ammonium chloride was removed and 10⁸ PFU of wildtype phages were added to spheroids for up to 24 h. Spheroids were completely dissociated with 0.25% trypsin-EDTA and washed to discard extracellular phages. Cells were subjected to freeze-thaw cycles for lysis and release of phage particles. Phages recovered from lysed cells were plated on BB4 E. coli cells for quantification of plaques. Data is presented as the mean+SEM of three independent experiments, carried out in triplicates. Differences in PFU between treated and untreated were not determined to be statistically significant by the Mann-Whitney test, p>0.05 [0136] Furthermore, to ensure that exposure to trypsin-EDTA was not going to deactivate phages, viability assays were also conducted on phage particles resuspended in different concentrations of trypsin-EDTA at 37° C. for 1 h (Table 7).

TABLE 7
Effect of Trypsin-EDTA on phage viability

| | | EOP of |
|---|---|---|
| | EOP of λF7 | D::EGF phages |
| EDTA-trypsin | incubated at | incubated at |
| concentration | 37° C.¹ | 37° C.¹ |
| 0% | 1.00 ± 0.01 | 1.00 ± 0.02 |
| 0.1% | 0.89 ± 0.03 | 0.87 ± 0.03 |
| 0.25% | 0.89 ± 0.07 | 0.89 ± 0.09 |
| 0.5% | 0.84 ± 0.07* | 0.79 ± 0.07** |
| 1.00% | 0.72 ± 0.10** | 0.76 ± 0.12* |

¹All EOPs have been expressed as a mean of three independent assays and determined using BB4 (SupE, SupF double suppressor) as the 100% plating control and plated at 37° C. Reductions in viability of D::EGFand wildtype phages were not statistically significant using the 2-way ANOVA for 0-0.25% trypsin EDTA.

The pre-treatment of spheroids with ammonium chloride on the yield of internalized phages was not determined to be significant. However, treatment enhanced the recovery of phages for up to 8 h, but appeared to be slightly detrimental in recovering phages after 24 h (FIG. 16). Pre-treatment of spheroids with ammonium chloride was carried out for the internalization assays (Table 8).

Phages were recovered from intercellular spaces of spheroids and intracellularly from lysed cells. Differences in phage recovery were compared between the wildtype and Hi-D::EGF phages after 0.5, 4, 8 and 24 h of treatment, to determine whether D::EGF-EGFR interactions would increase the percentage of internalized cells (FIG. 17). FIG. 17 shows percent internalization of phages determined from HT29 spheroids. The percentage of internalized phages that were associated with the spheroid was calculated from the data shown in Table 6 using the formula above. The display of EGF correlated with the mean percentage of intracellular phages particle intracellularly at all treatment periods. Percent internalization did not appear to be dependent on the density of EGF molecules displayed on the surface of phage. 2-way ANOVA multiple comparisons test indicated no statistically significant differences in the % internalization difference between WT phages and both D::EGF-phages p>0.05. To minimize possibly scoring extracellular phages that were bound to the surface of cells as intracellular, The cells were incubated with an acidic wash (pH 2) to inactivate the phages. However, this technique does not inactive 100% of phages, as such a background of extracellular bound phages is inevitable. Counts in Table 7 should be considered as relative. EOP's in Table 6 were corrected by taking account of the viability decrease at 37° C. (Table 3) as the 100% control using the formula:

$$adjusted EOP = \frac{PFU of inter \text{-} or intracellular phages}{{10}^{8} PFU \times percent viability}$$

PFU of recovered inter and intracellular phages were expressed as ‘percent internalization’ in FIG. 17 by using formula:

$$\frac{adj . EOP of intracellular phages}{\begin{matrix}adj . EOP of intracellular phages + \\ adj . EOP of intercellular phages\end{matrix}} \times 100 \%$$

TABLE 8
EGF display increases the recovery of phage particles from HT29 plasmids

| | | | EOP of | EOP of | | EOP of | |
|---|---|---|---|---|---|---|---|
| Duration of | | | WT | Lo-D::EGF- | | Hi-D::EGF- | |
| treatment | | | phage¹ | phage¹ | p⁴ | phage¹ | p |
| 30 | min | Intercellular² | 6.59E−04 | 1.27E−03 | 0.541 | 1.56E−03 | 0.296 |
| | | Intracellular³ | 5.17E−05 | 1.20E−04 | 0.779 | 1.30E−04 | 0.722 |
| 4 | h | Intercellular | 1.66E−02 | 4.10E−02 | 0.966 | 5.72E−02 | 0.438 |
| | | Intracellular | 7.62E−05 | 7.35E−05 | 1.000 | 3.92E−03 | 0.483 |
| 8 | h | Intercellular | 1.03E−03 | 1.71E−02 | 0.102 | 1.83E−02 | 0.081 |
| | | Intracellular | 6.36E−05 | 1.24E−03 | 0.273 | 1.47E−03 | 0.177 |
| 24 | h | Intercellular | 1.01E−03 | 7.15E−03 | 0.794 | 1.13E−02 | 0.475 |

| | | | | | |
|---|---|---|---|---|---|
| Intracellular | 7.77E−06 | 4.16E−04 | 0.477 | 3.12E−04 | 0.653 |

¹All EOPs were adjusted to take account of decrease in cell viability with prolonged incubation at 37° C. and are expressed as a mean of three independent assays + SEM, n = 3, and determined using BB4 (SupE, SupF double suppressor) as the 100% plating control.
²Phages recovered from the interstitial spaces of HT29 spheroids
³Phages recovered from lysed HT29 cells
⁴Statistically significant differences in expression were determined by two-way ANOVA Tukey's multiple comparisons test, p- values were compared to the WT phage as a control

The mean of recovered phages indicates that the uptake of phages by HT29 cells was enhanced by the display of EGF molecules; however, this was not determined to be statistically significant (FIG. 17). Large variances in the recovery of phages were expected as large titer of phages were initially added, and spheroids were manually washed to discard unassociated phages.

Therefore, relative trends in the association must be considered instead. Substantial differences in phage uptake between the D::EGF phages and wildtype phages was observed at 30 min. Recovery of both types of EGF-displaying phage particles from the interstitial spaces and within cells were greater by over 150-fold than wildtype phages (Table 8). This suggests that EGF may assist in the initial attachment of phages to the cells, resulting to a greater percentage of internalization. At 4 h, the number of phage particles associated with the spheroids extracellularly was similar for both Lo- and Hi-D::EGF phages (Table 8). However, the percentage of internalization was much higher for the Hi-D::EGF phages, suggesting a ligand density-dependent mechanism. After 8 h, average uptake of phage particles was at peak for both D::EGF and wildtype phages, although there were fewer phages associated with the spheroids than at 4 h. After 24 h of treatment, internalized phage particles were likely degraded in the cells even with the assistance of ammonium chloride (FIG. 16), and it is unclear whether the quantitation provided accurate representation of internalized phage particles.

### Example 5

### Effects of EGF Display on Bacteriophage on Spheroid Growth

To determine whether phages would interfere with spheroid growth, 5,000 HT29 cells were seeded on agarose and low (10⁴ PFU) or high (10⁸ PFU) concentrations of Lo-D::EGF, Hi-D::EGF and wildtype phages were added after 48 h to the partially-formed spheroids. EGF displayed on phages were expected to bind to the EGFR on the surface of HT29, blocking cells from receiving EGF and other growth signals, and consequently slowing the growth of HT29 spheroids.

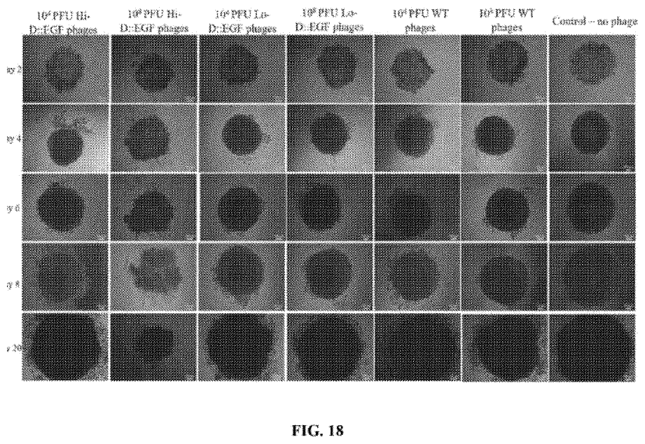

FIG. 18 shows the growth of partially-formed spheroids affected by the presence of phages. HT29 cells were seeded for 48 h to form partially-formed spheroids. 10⁴ or 10⁸ PFU of phages were added to each spheroid. The formation and growth of spheroids were monitored for 20 d with a bright-field microscope. Each picture is representative of 3 replicates of spheroids for each condition. There were no visible differences in the morphology of spheroids until about 2 d after the application of phages (4 d after seeding). Adding high concentration of Hi-D::EGF phages appeared to have interfered in the complete aggregation of cells, which was more apparent after 6 d of phage application (‘day 8’). By day 20, these spheroids treated with Hi-D::EGF phages appeared to be more dense and compact than they had been previously; they were approximately three-fold smaller in size than the control spheroids (Table 9). Adding a low concentration of Hi-D::EGF phages, however, did not result in the same pattern of growth inhibition in spheroids; these spheroids developed similarly in shape and size to those treated with wildtype phages. Spheroids treated with wildtype and Lo-D::EGF phages of both high and low concentrations exhibited similar sizes and density. These spheroids were slightly smaller than the control spheroids.

TABLE 9
HT29 spheroids treated with D::EGF phages are smaller on day 20

| Spheroid treated with: | Diameter (μm) |
|---|---|
| Control (no phage added)² | 1533 ± 47 |
| 10⁸ PFU Hi-D::EGF phges³ | 422 ± 28 |
| 10⁴ PFU Hi-D::EGF phges⁴ | 1329 ± 54 |
| 10⁸ PFU Lo-D::EGF phges⁵ | 1198 ± 19 |
| 10⁴ PFU Lo-D::EGF phges⁶ | 1509 ± 52 |
| 10⁸ PFU Wildtype phages⁷ | 1410 ± 35 |
| 10⁴ PFU Wildtype phages⁸ | 1473 ± 202 |

¹n = 3 ± SD and diameters were measured with the QCapture Pro software
²max = 1598, min = 1516
³max = 459, min = 389
⁴max = 1406, min = 1280
⁵max = 1218, min = 1172
⁶max = 1580, min = 1455
⁷max = 1459, min = 1377
⁸max = 1754, min = 1283

Effects of EGF-displaying phages on initial cell aggregation: Next, in order to determine whether Hi-D::EGF phages would interact with cell-surface receptors to disrupt initial cell aggregation, phages were co-seeded with 3,000 HT29 cells and were imaged with ⁷max=1459, min=1377 ry 2 d.

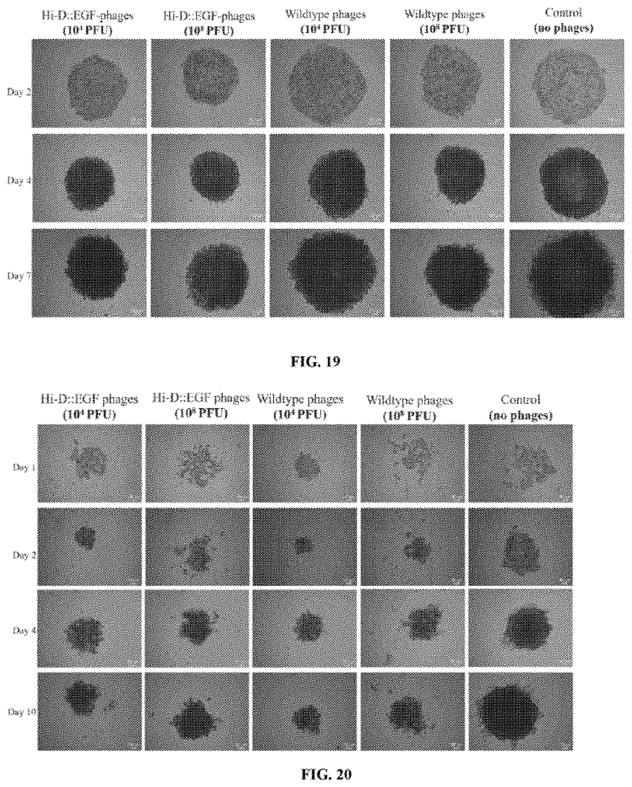

⁸max=1754, min=1283 HT29 cells co-seeded with D::EGF phages or wildtype phages. Effects of phage presence on initial cell aggregation to form spheroids were monitored. Representative spheroids are shown above of three replicates of spheroids. All images were taken on a Leica DM2000 microscope and QImaging Micropublwasher 5.0 RTV camera using the QCapture Pro 7 Software. As shown in FIG. 19, HT29 cells co-seeded with D::EGF phages and high concentration of wildtype were smaller and more compact than the control spheroids. Low concentration of wildtype phages did not appear to have imparted any visible effects in initial spheroid formation and was similar in morphology and size to the control spheroid on day 2. By day 4, cells treated with phages had formed smaller, compact, three-dimensional spheroids. The control spheroid without phages was greater in size, but cells appeared to be sparser and loosely-associated at the centre.

Phages may have settled to the agarose surface in FIG. 19, which would diminish the amount of phages interacting with HT29 cells. Therefore, phages were incubated with HT29 cells on ice for 3 h to allow initial cell-phage connections to form. Pre-chilling on ice for at least 1 h is a common approach for blocking cellular receptors with antibodies and ligands. Following this adsorption phase, the cell-phage mixture was seeded normally onto the agarose surface (FIG. 20).

FIG. 20 shows growth of pre-chilled HT29 cells with phages. 3,000 HT29 cells were incubated for 3 h on ice with 10⁴ or 10⁸ PFU of wildtype or D::EGF phages. Phages and cells were then co-seeded onto an agarose surface and grown for 10 d. Images are representative of 3 replicates of spheroids. All images were taken on a Leica DM2000 microscope and QImaging Micropublwasher 5.0 RTV camera using the QCapture Pro 7 Software. Unexpectedly, lower concentrations of both wildtype and D::EGF phages appeared to assist in the compact packing of the cells by day 2 compared to the control. Cells co-seeded with higher concentrations did not fully aggregate after the first 24 h, but fully assembled by day 4. At this stage, spheroids treated with phages did not adopt well-defined shapes. The high concentration of phages adhered to the surface of cells may have obstructed the cells from fully forming cell-cell connections. Additionally, this may have prevented cell-signaling molecules from binding to their surface-receptors, inhibiting growth and proliferation. There were no discernible differences between cells treated with wildtype phages and D::EGF phages during the initial process of cell aggregation in FIG. 20. Cell proliferation appeared to be much more accelerated in the control spheroid after 10 d.

Effects of phages on fully-formed HT29 spheroids: In order to determine whether phages can infiltrate HT29 spheroids and induce visible changes in spheroid morphology, 5-d old HT29 spheroids were treated with high and low concentrations of wildtype and D::EGF phages.

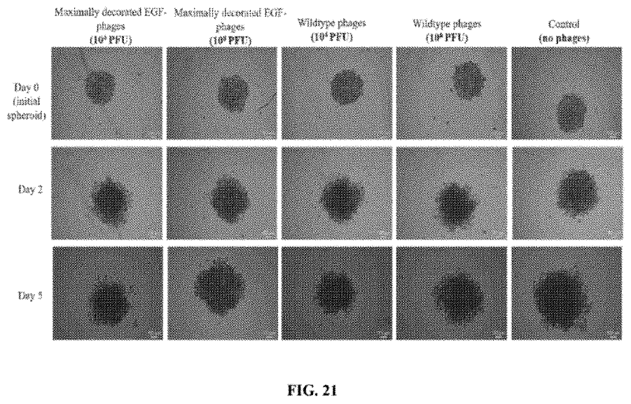

Cell proliferation continued to occur despite the presence of phages (FIG. 21, Images are representative of 3 replicates of spheroids. All images were taken on a Leica DM2000 microscope and QImaging Micropublwasher 5.0 RTV camera using the QCapture Pro 7 Software.). 2 d after adding phages to the spheroids, there were no visible differences to the control spheroid. 5 d after adding both types of phages, the lag in cell proliferation was noticeable when compared to the control. The spheroid treated with a high concentration of Hi-D::EGF phages exhibited sparse areas between cells. These cells appeared to have dissociated from neighbouring cells, contributing to a larger diameter overall. D::EGF phages may have bound to EGFR on the surface of the cells, reducing the overall cell-cell contacts holding the spheroid together.

### Example 6

### Cytotoxicity of D::EGF Phages to 11T29 Cells

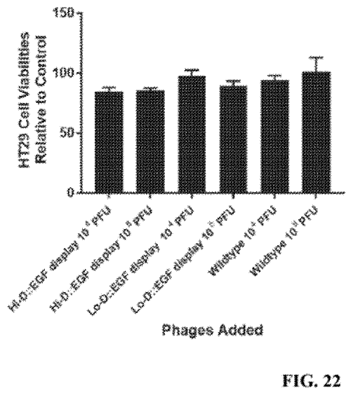

The inventors sought to test the level of EGF-phages and wildtype phages on cell viability and performed an MTT assay. Cells were exposed to low (10⁴) or high (10⁸) PFU of phages for 24 h and HT29 cells cultured in serum-containing medium were used as the 100% viable control (FIG. 22). FIG. 22 shows HT29 cell proliferation (MTT) assay in response to phage treatment relative to control. Cells were seeded into the wells of a 96-well plate in 9 replicates for each treatment group. Cells were cultured normally for 72 h before the addition of phages. Cells were then exposed to phages for 24 h. At the end of this period, an MTT assay was performed to assess cellular viability. Cells cultured normally with serum were considered 100% viable and all percentage of cell viabilities above are expressed relative to the 100% control. The results shown represent three independent experiments. The relative viabilities of monolayer untreated HT29 versus HT29 treated by phages were not statistically different by a Mann-Whitney test. The percentages of viability for all conditions were expressed relative to the 100% viable control. At the end of the 24 h exposure to phages, cell viabilities were only marginally reduced; there was approximately 15% reduction in HT29 cell viability in response to Hi-D::EGF phages, of both concentrations. The differences in viability in response to both D::EGF phages and wildtype phages were not determined to be significant.

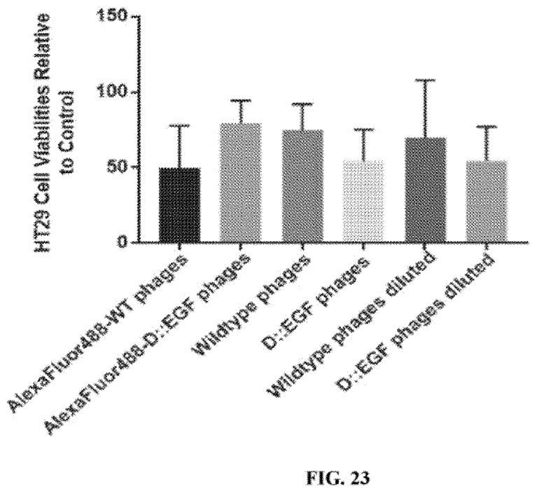

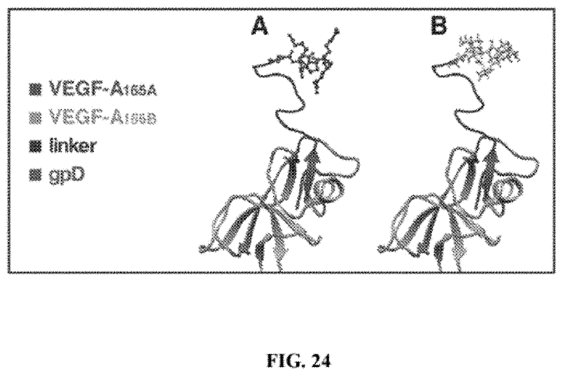

Cytotoxicity induced by phages after a 24 h exposure was also measured in spheroids using the CellTiter-Glo® 3D cell viability assay (FIG. 23). FIG. 23 shows HT29 spheroid cell viabilities relative to control in response to phage treatment. 7-day old HT29 spheroids (seeding density: 5,000 cells) were treated with wildtype phages, Hi-D::EGF phages or AlexaFluor488 tagged phages for 24 h. Cell viability of HT29 spheroids treated with phages were assessed by measuring ATP content and the luminescence output via the luciferase enzyme supplied in the ultra-Glo™ reagent. All viabilities are expressed as relative values to spheroids cultured normally with serum, which were considered to be 100% viable. The results shown represent four replicates. The relative viabilities of spheroids untreated HT29 versus HT29 treated by phages were not statistically different by multiple comparisons one-way ANOVA test. This assay tested the amount of ATP, which is proportional to the number of viable cells in 3D culture. HT29 spheroids were grown, treated with 10⁸ PFU of wildtype and D::EGF phages for 24 h, and were assayed for cell viability.

The mean cell viability was higher for HT29 spheroids exposed to wildtype phages than D::EGF phages (74% versus 54%). This result correlates to the brightfield images of HT29 spheroids that were smaller in size in response to D::EGF phages than wildtype phages (FIG. 18-21). The mean cell viabilities were similar between spheroids treated with diluted and undiluted wildtype and D::EGF phages. However, the results were reversed for phages following AlexaFluor488 tagging—spheroids applied with D::EGF phages had 79% viability, versus 49% for wildtype phages. Percentage of cell viability was analyzed by the multiple comparisons one-way ANOVA test, however, these results were not deemed statistically significant (p<0.05).

Bacteriophage λ accumulates in multicellular spheroids: The NIH3T3 spheroids disclosed in the examples are relatively small, approximately 300 μm in diameter, and lacking the hypoxic regions exhibited by large, solid tumours. Despite the small size, NIH3T3 spheroids provided a proof-of-concept for native interactions between λ phages and mammalian cells in the context of 3D culture, specifically for assessing the ability for adherence and penetration of the intercellular spaces. Fibroblasts are the most common type of cells of the connective tissue and synthesize the ECM, forming a structural network that is difficult to infiltrate in tissues. Confocal images showed the propensity of λ phages to adhere and accumulate between the fibroblasts. This implicates that phages may be able to penetrate through the network of connective tissues interposed between malignant cells and normal host tissues, which often structurally prevents the effective infiltration of nanoparticles.

As all spheroids were thoroughly washed to discard unbound/weakly-bound phages, the phage-associated fluorescence emitted on the surface of the spheroids evidences surface interaction between phages and fibroblasts. This is in accord with other findings arguing for inherent phage adherence to cancer cells in vivo and in vitro. Green fluorescence associated with wildtype phages was observed throughout various cross-sections of NIH3T3 spheroids. Phage adherence appeared to be time dependent; this was observed as early as 30 min, whereby small fluorescent green dots could be visualized at the rim of spheroids (FIG. 10). A major limitation of the confocal microscope was the inability to visualize dense inner regions of the spheroids. FIG. 10 shows evenly-distributed phage-associated fluorescence throughout visible regions in the optical sections at 24 h, rather than simply at peripheral cells of spheroids as seen at 6 h. At 24 h, phages have likely localized both inter- and intracellularly; while the exact nature of this interaction is unknown, this increase in positive signal over time, both in intensity and the areas covered, suggests a deeper infiltration and internalization of phages into the spheroids. More phages were likely able to accumulate on the surface of cells, as bound phages diffused inwards into the spheroid, freeing up the surfaces of the cells for binding by phages. This would result in prevailing green fluorescence throughout the cross-sections as seen in FIG. 10.

Next, using the dual control phage display system, maximally and minimally decorated D::EGF phages were generated and applied to HT29 spheroids. These spheroids were consistently robust, possessing diameters of −350 μm. As expected, D::EGF phages demonstrated greater accumulation within spheroids at 8-24 h than wildtype phages, which suggests that λ-EGF-EGFR association mediated this interaction (FIG. 12-14). However, wildtype phages appeared to have interacted with the spheroids at an earlier time point of 4 h (FIG. 12, 13). This could be attributed to the overall bulkiness of the D::EGF phages; phages suspended in solution must rely on Brownian motion to come into contact with spheroids. Wildtype phages, which do not carry an additional load, may be able to diffuse more rapidly to interact with the cells. Prior study by Nicastro et al. compared sizes of λF7 phages displaying eGFP molecules under the permutations of various E. coli suppressor hosts and temperature by dynamic light scattering; phages grown on SupD at 37° C. (maximal display) was about 3 times greater in diameter than wildtype phages, which are approximately 62 nm in diameter and 150 nm in length. Phages grown on SupF at 37° C. (minimal display) was about 1.6 times greater than the wildtype phages.

Interestingly, wildtype phages appeared to have a better accumulation within NIH3T3 spheroids than HT29 spheroids, specifically at 24 h (FIG. 10-13). This may be caused by differences in cell-cell contacts found in different cell types. Epithelial cells are characterized by tight and adherence junctions that hinder the passage of molecules through the paracellular spaces between adjacent cells. For example, this disclosure showed 400-500 um human cervical carcinoma (SiHa) spheroids consisted of tight junctions connecting neighbouring cells that restricted movement of materials, which was not observed with spheroids of other cell lines. Furthermore, ECM creates a physical barrier to prevent intratumoural drug penetration, and its composition can vary among different cell lines. Spheroids required collagenase treatment for effective penetration of nanoparticles greater than 100 nm. Hence, HT29 spheroids may consist of greater percentage of collagen or other ECM components than NIH3T3 spheroids, obstructing the penetration and accumulation of phages.

EGF display enhances bacteriophage uptake into HT29 cells: Monoclonal antibodies that target the extracellular domain of EGFR, such as cetuximab, are internalized with EGFR as antibody-receptor complexes. During this process, the receptor dimerizes, but downstream signaling events are not triggered. Similar cellular events were expected upon binding of D::EGF phages to EGFR. Certain green fluorescent particles appeared more yellow and diffuse in the confocal images. This suggested intracellular fluorescence from λ phages internalized by the HT29 cells (FIG. 15). CellTracker-cytosolic labeling does not obscure the green fluorescence emitted by internalized phages.

The recovery of D::EGF phages both intercellularly and intracellularly were greater than wildtype phages by almost two-folds at 30 minutes. This is in agreement with the confocal images showing phage localization (Table 7, FIG. 12-14), which is consistent with that EGF display enhances the phage adherence to the HT29 cells. 30 min incubation in FIG. 17 may be the most representative time point to compare the differential rate of cellular uptake between D::EGF phages and wildtype phages, without the influence of intracellular phage degradation.

EGF-displaying bacteriophage λ slows spheroid growth and HT29 cell aggregation: EGF molecules displayed on λ phages likely bound to the EGFR on HT29 cells, preventing the downstream signaling events that promote spheroid growth. Monoclonal antibodies that bind to EGFR have been shown to successfully inhibit growth and induce apoptosis of HT29 by competitively inhibiting the binding of endogenous ligands and EGF-induced tyrosine kinase-dependent EGFR phosphorylation. Similarly, high concentration of maximally-decorated D::EGF phages appeared to slow cell proliferation and prevent complete cell-cell connections from forming, as shown in FIG. 18. Interestingly, this did not appear to be ligand density-dependent, as minimally-decorated D::EGF phages (Lo-D::EGF phages) did not influence spheroid growth and formation. Avidity of the receptor-ligand interaction between phages and cells may be a possible explanation. To conceptualize this, the larger size of each maximally-decorated D::EGF phage particle may enable the interaction with many cells simultaneously through greater density of EGF molecules. This would entrap phage particles between each cell, forming lattice-like structures composed of phages interposed between HT29 cells via EGF-EGFR connections. This would effectively and simultaneously block EGFRs on multiple cells, impeding any growth factors from reaching the receptors. Eventually, the rate of cell proliferation would overcome the EGFR block, as observed from later days.

All spheroids treated with phages, including wildtype phages, exhibited smaller spheroids by day 20 compared to the control (FIG. 18), most likely due to surface-bound phages blocking the potentiation of cell-cell interactions. This effect was also apparent when phages were co-seeded with cells (FIGS. 19 & 20). It is contemplated that phage internalization via polysialic acid will induce biological changes in the cell because the turnover of cell surface polysialic acid will reduce the numbers of available on the surface, reducing its role in regulating cell interactions. Similarly, it is expected that application of phages, which are subject to internalization, will induce changes in cell activities and ultimately affect spheroid formation.

The signaling kinetics and the alteration of downstream effects to the treatment with D::EGF phages and their subsequent binding to EGFR on HT29 cells can only be speculated. It is likely that multiple cellular mechanisms are altered in response to the D:EGF-EGFR reaction. Cell proliferation appears to have slowed, which may be a result of the inactivation or reduced signaling of the Ras/MAPK pathway. This pathway is normally induced upon EGFR phosphorylation and the binding of Grb2 and Src homology 2. Reduced cell survival is also implicated with the cells treated with D::EGF phages in FIG. 18, which suggests the involvement of EGFR in the PI(3)K/Akt pathway through adaptor protein Gab-1. HT29 cell adhesion appears to also be lost in response to D::EGF phages as shown in FIGS. 19 & 22, which may be a result of the loss in the ability of STAT3 from binding to the activated EGFR. Consequently, this would prevent STAT3 dimerization and translocation into the nucleus to regulate gene transcription to maintain epithelial cell polarity and adhesion. These results were not anticipated, as HT29 colon adenocarcinoma cells harbour the V600E BRAF heterozygotic mutation, which is often responsible for resistance (8.3%) to anti-EGFR therapy. Mutations in BRAF V600E can allow for downstream signaling events to persist in the absence of signaling molecules, leading to constitutive cell survival and proliferation. However, this study has demonstrated that phages with a maximal display of EGF molecules will slow spheroid growth, most likely through the reduced cell proliferation and survival. The same effects were not exhibited when spheroids were treated with wildtype phages. Bacteriophage λ is not cytotoxic to HT29 cells: MTT assay (FIG. 22) did not show significant differences in the viability of monolayer HT29 cells in response to phage application. D::EGF phages did not induce cytotoxicity in the monolayer culture. In the 3D culture, cell viability was expected to be different as gene expression profiles are often different from monolayer cultures (FIG. 23) Wildtype phages did not significantly reduce the viability of HT29 spheroids after 24 h. D::EGF phages, however, reduced the mean of number of viable cells to 54% (FIG. 23). These results are in agreement with the brightfield images (FIG. 18-21); spheroids demonstrated loose cell-cell connections and were smaller when treated with D::EGF phages. These spheroids consisted of fewer cells compared to the control in later stages, suggesting a slower cell proliferation and/or cell death (FIG. 18). 100-fold diluted mixtures of both D::EGF and wildtype phages resulted in similar levels of toxicity for the cells as the undiluted mixtures. The dilution of phages used in this experiment may not have been high enough to see correlating reduction in cytotoxicity as toxicity levels could plateau at certain concentrations of phages.

The inventors have demonstrated the therapeutic potential of phages against solid tumours as λ phages were able to interact with mammalian cells and accumulate in both spheroids and in cells. Phages can be genetically modified to display a targeting molecule on the surface for specificity; this can also slow growth by binding to plasma membrane receptors to induce/inhibit signalling events that result in changes in gene expression and cell growth regulation.

The number of applications for phages is growing in the field of cancer biology as well as the interest in developing a phage-based drug delivery system; however, the ability of phages to penetrate tightly packed cells in the tumour microenvironment has not previously been shown. In this disclosure, wildtype phages without any surface modifications and EGF-displaying phages have been compared for the adherence and penetration into HT29 colon adenocarcinoma spheroids, cellular uptake and impacts on spheroid growth. λ phages did not require ligand mediation to adhere to the surface of spheroids, although the display of EGF peptides on λ phages increased the level of phage-associated fluorescence on the surface of HT29 spheroids. Visualization of phages localized in the inner regions of spheroids was limited by confocal microscopy; however, phage-associated fluorescence was diffuse in all regions that were visible after 24 h in fibroblast spheroids. The significance of effective phage penetration and accumulation into spheroids is its potential to successfully traverse the dense tumour microenvironment composed of fibroblasts, immune cells and ECM, and potentially resulting in better biodistribution than other nanoparticles.

EGF-displaying phages showed higher propensity to be internalized by the HT29 cells than wildtype phages. An implication for lambda phage internalization into mammalian cells is the potential for successful targeting of phages for gene delivery. However, one of the limiting steps of phages for gene delivery is nuclear localization, as phages are most likely routed to the lysosomes upon internalization

Bacteriophages are highly immunogenic and are readily cleared by the reticuloendothelial system within a few hours of entry into systemic circulation. Bacteriophage (prokaryotic) DNA is enriched with pathogen-associated molecular patterns (PAMPs) that occur at ˜20-fold greater frequency than in eukaryotic DNA. These PAMPs consist of unmethylated CpG dinucleotide motifs that are recognized by specific Pattern Recognition Receptors in the endosome of immune cells (particularly Macrophages, dendritic cells and B-cells that present antigen to T-cells) called Toll-like Receptor (TLR) 9. TLR-9 cascade signaling results in potent pro-inflammatory and cytokine response production that stimulates cell-mediated immunity (Th1 response). The proinflammatory response has not been recorded as harmful but rather serves as an adjuvant to recruit immune cells and antigen presenting cells to the site of recognition. This can be particularly important in the tumour microenvironment which tends to be immunosuppressed. Bacteriophages within the tumor microenvironment may serve as adjuvants that may be endocytosed by antigen presenting cells and stimulate Th1 response. Th1 immune responses of cell-mediated immunity and inflammation are necessary for the attack and elimination of tumours and their genesis. Since bacteriophage serve as a polarizing adjuvant, EGFR targeting of bacteriophage to the tumour microenvironment of an EGFR-positive tumour could serve to stimulate a strong cell-mediated and inflammatory response in the tumour microenvironment, thereby inhibiting tumour growth and/or disease progression.

Lastly, the application of EGF-displaying phages in high concentrations showed interference in cell aggregation for spheroid formation, slowing its growth. This was most likely caused by the blocking of EGFR on HT29 cells and depriving the cells of growth factors; this exposure to phages was shown to result in less viable cells in the spheroid. These results highlight the utility of bacteriophages presenting EGFR-binding moieties on their cell surface for therapeutic applications including treating cancer.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby. The scope of the claims should not be limited by the particular embodiments set forth herein, but should be construed in a manner consistent with the specification as a whole.

### REFERENCES

1. Arab et al. 2018 Lambda phage nanoparticles displaying HER2-derived E75 peptide induce effective E75-CD8+ T response. Immunol Res 66(1):200-206.
2. Bachmann (1972) Pedigrees of some mutant strains of Escherichia coli K-12. Bacteriol Rev 36(4):525-57.
3. Ciardiello et al, 2000, Antitumor effect and potentiation of cytotoxic drugs activity in human cancer cells by ZD-1839 (Iressa), an epidermal growth factor receptor-selective tyrosine kinase inhibitor. Clin Cancer Res. 2000 6(5):2053-63.
4. Dreher et al. 2006 Tumor vascular permeability, accumulation, and penetration of macromolecular drug carriers. J Natl Cancer Inst 98(5):335-44.
5. Gottesman, 2002, Mechanism of cancer drug resistance. Annu Rev Med 53:615-27.
6. Hajitou et al., 2007 Design and construction of targeted AAVP vectors for mammalian cell transduction. Nat Protoc. 2(3):523-31.
7. Handbook of Pharmaceutical Excipients, 5th Edition; Rowe et al., Eds., The Pharmaceutical Press and the American Pharmaceutical Association: 2005.
8. Handbook of Pharmaceutical Additives, 3rd Edition; Ash and Ash Eds., Gower Publishing Company: 2007.
9. Inchley, 1969 The activity of mouse Kupffer cells following intravenous injection of T4 bacteriophage. Clin Exp Immunol. 5(1):173-87.
10. Lech, Reddy, & Sherman, 1990 Preparing Lambda DNA from Phage Lysates. Current Protocols in Molecular Biology, Chapter 1.
11. Maruyama et al. (1994) Lambda foo: a lambda phage vector for the expression of foreign proteins. Proc Natl Acad Sci USA 91(17):8273-7.
12. Matsuo et al. 2011 Analysis of the anti-tumor effect of cetuximab using protein kinetics and mouse xenograft models. BMC Research Notes 4:140.
13. Mikawa el al. (1996) Surface display of proteins on bacteriophage lambda heads. J Mol Biol. 262(1):21-30.
14. Mikhail et al., 2013 Multicellular tumor spheroids for evaluation of cytotoxicity and tumor growth inhibitory effects of nanomedicines in vitro: a comparison of docetaxel-loaded block copolymer micelles and Taxotere®. PLoS One 8(4).
15. Merril et al. 1996. Long-circulating bacteriophage as antibacterial agents. Proc Natl Acad Sci USA 1996 93(8):3188-92.
16. Netti et al. 2000 Role of extracellular matrix assembly in interstitial transport in solid tumors. Cancer Res 60(9):2497-503.
17. Nguyen et al., 2017. Bacteriophage Transcytosis Provides a Mechanism To Cross Epithelial Cell Layers 8(6).
18. Nicastro et al., 2013; Appl Microbiol Biotechnol. 97(17):7791-804. Construction and analysis of a genetically tuneable lytic phage display system.
19. Pharmaceutical Preformulation and Formulation, Gibson Ed., CRC Press LLC: Boca Raton, Fla., 2004.
20. Przystal et al., 2013. Proteasome inhibition in cancer is associated with enhanced tumor targeting by the adeno-associated virus/phage Mol Oncol 7(1):55-66.
21. Remington: The Science and Practice of Pharmacy, 21st Edition; Lippincott Williams &amp; Wilkins: Philadelphia, Pa., 2005.
22. Smith, T. L., et al. 2017. An AAVP-based solid-phase transducing matrix for transgene delivery: Potential for translational applications. Cancer Gene Therapy, 24(8), 358-360.
23. Sokolenko et al. 2012. Graphical analysis of flow cytometer data for characterizing controlled fluorescent protein display on λ phage. Cytometry A, 81(12):1031-9.
24. Soothill et al., 1994 Bacteriophage prevents destruction of skin grafts by Pseudomonas aeruginosa. Burns 20(3):209-11.

What is claimed is:

1. A pharmaceutical composition comprising a plurality of recombinant bacteriophage engineered to present an epidermal growth factor receptor (EGFR)-binding moiety on the bacteriophage cell surface together with a pharmaceutically acceptable excipient, diluent, or carrier, wherein the EGFR-binding moiety is for binding the extracellular domain of an EGFR.

2. The pharmaceutical composition of claim 1, wherein the plurality of bacteriophage is a plurality of lytic bacteriophage.

3. The pharmaceutical composition of claim 2, wherein the plurality of bacteriophage is a plurality of lamboid phage, T4 or T7 bacteriophage.

4. The pharmaceutical composition of claim 3, wherein the plurality of bacteriophage is a plurality of λ bacteriophage.

5. The pharmaceutical composition of claim 4, wherein the plurality of bacteriophage is a plurality of λF7 bacteriophage.

6. The pharmaceutical composition of claim 1, wherein the EGFR-binding moiety is EGF or a functional variant thereof.

7. The pharmaceutical composition of claim 6, wherein the EGF comprises the amino acid sequence as set forth in SEQ ID NO:1 or a variant thereof, wherein the variant thereof comprises an amino acid sequence that is at least 90% identical, such as at least 95%, 98% or 99% identical to the sequence of SEQ ID NO:1 across the full length thereof.

8. The pharmaceutical composition of claim 1, wherein a titration of the bacteriophage in the composition ranges from about 10⁷ PFU per ml of total volume of the composition to about 10¹⁰ PFU per mL of total volume of the composition.

9. The pharmaceutical composition of claim 1, wherein the bacteriophage are in an amount sufficient to reduce the growth of an EGFR-positive tumor.

10. The pharmaceutical composition of claim 1, wherein the EGFR-binding moiety is present on the bacteriophage cell surface in an amount sufficient to target the bacteriophage to an EGFR-positive tumor.

11. The pharmaceutical composition of claim 10, wherein the EGFR-binding moiety is present on the bacteriophage cell surface in an amount sufficient to target the bacteriophage to a tumor microenvironment of an EGFR-positive tumor.

12. The pharmaceutical composition of claim 1, wherein the pharmaceutical composition is formulated for intravenous administration, intratumoral administration or rectal administration.

13. The pharmaceutical composition of claim 1, wherein the plurality of recombinant bacteriophage are prepared using a lytic phage display system.

14. The pharmaceutical composition of claim 1, wherein the EGFR-binding moiety is capable of:
inhibiting dimerization of an EGFR to an Erb family member;
competing with EGF for binding of an EGFR;
modulating one or more EGFR downstream signaling pathways; or
promoting internalization of an EGFR into a cell.

15. A bacteriophage for infiltrating a tumor microenvironment of an epidermal growth factor receptor (EGFR)-positive tumor, the bacteriophage comprising a polypeptide, said polypeptide comprising:
a targeting moiety for directing said bacteriophage to at least one target molecule expressed by said at least one target cell,
wherein the targeting moiety is an EGFR-binding moiety and is present on the surface of the bacteriophage in an amount sufficient for targeting the bacteriophage to the tumor microenvironment of an EGFR-positive tumor, the target molecule is EGFR and the at least one target cell is an EGFR-expressing tumor cell within the tumor microenvironment of the EGFR-positive tumor.

16. A method of treating a tumor in a subject in need thereof, the method comprising:
administering to the subject a pharmaceutical composition comprising a plurality of bacteriophage engineered to present an epidermal growth factor receptor (EGFR)-binding moiety on the bacteriophage cell surface, the EGFR-binding moiety for binding the extracellular domain of an EGFR, together with a pharmaceutically acceptable excipient, diluent, or carrier, in a dose effective to treat the tumor, wherein the tumor is an EGFR-positive tumor.

17. The method of claim 16, wherein treating the tumor comprises reducing a growth of the tumor.

18. The method of claim 16, wherein the tumor is a breast, lung, colon, stomach, pancreas, ovary, cervix, brain, head, neck, head and neck, prostate or kidney tumor.

19. The method of claim 16, wherein the tumor has a genetic signature comprising wildtype KRAS and mutated BRAF.

20. The method of claim 17, wherein the dose is effective to increase a penetration of the plurality of bacteriophage into the tumour microenvironment.
